(19) United States
(12) Patent Application Publication
(10) Pub. No.: US 2023/0210906 A1
(43) Pub. Date: Jul. 6, 2023

(54) Composite 3D Brain Organoids with Optic Structures, Uses Thereof and Culture Medium for Obtaining Them

(71) Applicants: Jay GOPALAKRISHNAN, Dusseldorf (DE); Elke GABRIEL, Mayschoss (DE); Aruljothi MARIAPPAN, Chorweiler (DE); Anand RAMANI, Rostock (DE)

(72) Inventors: Jay GOPALAKRISHNAN, Dusseldorf (DE); Elke GABRIEL, Mayschoss (DE); Aruljothi MARIAPPAN, Chorweiler (DE); Anand RAMANI, Rostock (DE)

(21) Appl. No.: 17/570,266

(22) Filed: Jan. 6, 2022

(51) Int. Cl. A61K 35/30; C12N 5/0793; C12N 5/079; C12Q 1/02

(52) U.S. Cl. CPC A61K 35/30; C12N 5/0619; C12N 5/0621; C12N 5/062; C12Q 1/025; C12N 2506/45; C12N 2513/00; C12N 2501/385; C12N 2501/155; C12N 2501/15; C12N 2501/16

(57) ABSTRACT

The present invention relates to 3D brain organoids, uses thereof, methods and culture medium for generating such organoids. An aspect of the invention provides brain organoids and methods of generating such organoids with bilaterally symmetric optic vesicles, containing both neuronal and non-neuronal cell types, and exhibiting functional circuitry. These organoids can be generated within short time intervals (e.g., 50 days) and therefore are useful for medical modelling and applications.

### FIELD OF THE INVENTION

The present invention relates to 3D brain organoids, uses thereof and culture medium for obtaining them.

### BACKGROUND OF THE INVENTION

Three-dimensional (3D) human brain organoids derived from induced pluripotent stem cells (iPSCs) provide unprecedented opportunities to simplify the complexity of brain development and diseases. Brain organoids have helped understand cellular diversities, complex interactions, and neuronal networks. Studies in the 18th century by Pander revealed that during embryogenesis, the retinal anlage develops laterally from the diencephalon of the forebrain, protruding as an optic vesicle. Huschke then showed that the distal part of diencephalon invaginates to assemble the optic vesicle. In the current century, pioneering works by the Sasai laboratory have demonstrated the remarkable ability of mouse and human pluripotent cells to undergo complex morphogenesis of the retinal anlage that contains cell types derived from neuroectoderm.

However, in vivo, optic vesicles constitute neuroectoderm and surface ectoderm-derived cell types that include specialized neuronal cell types of photosensitive rods and cones, retinal pigment epithelium (RPE), and non-neuronal cell types of lens and cornea. Although these structures have been difficult to reconstruct, elegant studies have demonstrated the ability of pluripotent cells to form these cell types individually (Eldred, K. C. et al. Thyroid hormone signaling specifies cone subtypes in human retinal organoids. Science 362, (2018)). Optic vesicles develop from the 4-5th post-ovulatory week with lens vesicle, cornea, and melanin granules (RPE) further continue in the 6th week with retinal differentiation. Interestingly, an immature retina-like structure could be observed in cerebral organoids. However, no mature retina has been described so far. Moreover, several tissues (including lens and cornea) have not been available from brain organoids so far.

### OBJECTIVE OF THE PRESENT INVENTION

It is an object of the present invention to provide brain organoids with mature retina-like structures. It is another object of the present invention to provide an optic vesicle 3D brain organoid. It is another object of the present invention to provide an 3D brain organoid comprising a lens and/or cornea.

It is another object of the present invention to provide brain organoids that assemble bilaterally symmetric optic vesicles in a topographically restricted manner mimicking embryogenesis. It is another object of the present invention to provide brain organoids that have functionally integrated optic vesicles allowing interorgan interactions to occur within a single organoid. It is another object of the present invention to provide organoids that contain physiologically active photoreceptors capable of recovering light sensitivity after photobleaching. It is another object of the present invention to provide complexity and interactions between different cell types present in optic vesicles that are functionally integrated within an organoid.

It is another object of the present invention to provide an optic vesicle 3D brain organoid that can be used to model ZIKV exposure during eye development. It is another object of the present invention to provide a method for fast generation of 3D brain organoids such as optic vesicle 3D brain organoids. It is another object of the present invention to provide an efficient and low-cost method for generation of 3D brain organoids. It is another object of the invention to provide artificial tissues or patches comprising cells derived from a brain organoid.

It is another object of the invention to provide an (artificial) retinal pigment epithelium (RPE). It is another object of the invention to provide an (artificial) source of photoreceptors (rods and/or cones). It is another object of the invention to provide an (artificial) source of Muller cells, ganglion cells, amacrine cells, bipolar cells, and/or horizontal cells. It is another object of the invention to provide an (artificial) lens or parts thereof. It is another object of the invention to provide an (artificial) cornea or parts thereof.

It is another object of the invention to provide treatment for diseases associated with visual impairment including retinal disorders. It is another object of the invention to provide a treatment for diseases associated with visual impairment. It is another object of the invention to provide a treatment for neurological disorders. It is another object of the invention to provide improved diagnosis, treatment, and/or prophylaxis of said diseases. It is another object of the invention to provide a treatment for Meesmanns Corneal Dystrophy. It is another object of the invention to provide a treatment for Best vitelliform macular dystrophy (BVMD). It is another object of the invention to provide a treatment for nonsyndromic congenital retinal nonattachment. It is another object of the invention to provide a treatment for posterior column ataxia with retinosa pigmentosa (AXPC1).

It is another object of the invention to provide toxicity in vitro assays including bacterial and viral infection tests. It is another object of the invention to provide approaches for modelling retinopathies and eye development. It is another object of the invention to provide drug screening in vitro assays.

### SUMMARY OF THE INVENTION

The present invention provides brain organoids with bilaterally symmetric optic vesicles, containing both neuronal and non-neuronal cell types, and exhibiting functional circuitry. This has been completely unexpected in view of the prior art. Furthermore, it is a great advantage of the present invention that these organoids can be generated within short time intervals (e.g. 50 days). The organoids according to the present invention are useful in several medical indications as explained in detail hereinafter.

In an embodiment, the invention relates to [1] a three-dimensional (3D) brain organoid characterized in that the 3D brain organoid contains both first cells that are neuronal and second cells that are non-neuronal.

In an embodiment, the invention relates to [2] a method for producing a 3D brain organoid characterized in that the 3D brain organoid contains both first cells that are neuronal cells and second cells that are non-neuronal, wherein the method comprises the steps of

i. culturing at least one stem cell,

ii. subjecting said at least one stem cell to neural induction leading to neurospheres,

iii. collecting and culturing the neurospheres,

iv. exposing said neurospheres to a composition comprising at least one retinoic acid receptor (RAR) activator, at least one bone morphogenetic protein (BMP) pathway inhibitor and at least one transforming growth factor (TGF) β/activin/nodal pathway inhibitor,

wherein the at least one stem cell comprises at most 100,000 cells, preferably between 10 and 90,000 cells, more preferably between 100 and 70,000 cells, even more preferably between 1,000 and 50,000 cells, more preferably between 5,000 and 25,000 cells, and wherein said exposure is made within two weeks, preferably within 2 days to 15 days, more preferably within 3 days to 14 days, even more preferred within 5 to 12 days after neural induction.

In an embodiment, the invention relates to [3] 3D brain organoids obtained by the method of [2] or tissue, preferably an artificial retinal pigment epithelium (RPE), isolated therefrom.

In an embodiment, the invention relates to [4] a patch comprising a tissue, preferably an artificial RPE, according to [3] and a basement membrane.

In an embodiment, the invention relates to [5] 3D brain organoid according to the embodiment of [1] or obtained by the method of [2] or tissue derived therefrom or cells derived therefrom for use in the treatment of diseases associated with visual impairment including retinal disorders, the 3D brain organoid or the tissue or the cells preferably being comprised in a pharmaceutical composition.

In an embodiment, the invention relates to [6] use of the 3D brain organoid according to the embodiment of [1] or obtained by the method of [2] in toxicity in vitro assays including bacterial and viral infection tests.

In an embodiment, the invention relates to [7] composition comprising at least one retinoic acid receptor (RAR) activator, at least one bone morphogenetic protein (BMP) pathway inhibitor and at least one transforming growth factor (TGF) β/activin/nodal pathway inhibitor.

### BRIEF DESCRIPTION OF THE DRAWINGS

FIG. 1 illustrates the generation of optic vesicle 3D brain organoids from human iPSCs. Panel A shows a schematic of human embryonic nervous system development. Panel B shows 30 day old organoids displaying the early occurrence of dark pigments. Panel C shows organoids displaying RAX-positive primordial eye field and NRL-positive photoreceptor progenitors. Panel D shows 30 day old organoids showing forebrain markers Pax6 and FoxG1- (green, right) containing ventricular zones. Panel E shows macroscopic views of 50-day-old organoids with each containing bilaterally symmetric dark pigmented areas. Panels F and G show bar diagrams quantifying organoids with pigmented optic vesicles and percentages of two-, one- or no optic vesicles-containing organoids derived from donor 1 (IMR90) iPSC cells.

FIG. 2 illustrates optic vesicle brain organoids displaying stratified neural retina. Panel A shows a transverse section through an optic vesicle of an organoid shows early tissue stratification beginning from RPE cell layer. Panel B shows a section through the RPE cell layer of an organoid. Panel C shows a sagittal view of RPE imaged by scanning EM reveals tightly packed single layer RPE cells. Panel D shows transmission EM of an RPE cell with numerous melanosomes and a primary cilium. Panels E and F show serial section of developing photoreceptor cilium.

FIG. 3 illustrates optic vesicle brain organoids displaying mature neurons. Panels A and B show sections of the whole organoid stained for Synapsin and Laminin, proteins essential for the release of synaptic vesicles at the presynaptic terminals, and polarization and layering of the neurons in the cortical region. Panels C and D show cortical region of a 3D brain organoid exhibiting a CTIP2 positive cells layer and representative images. Panels E and F show enrichment plots of cell cycle processes down-regulated in optic vesicle brain organoids and cycle division down regulated in matured optic vesicle brain organoids. Panels G and H display enrichment plots showing that mitosis genes are down regulated and that optic vesicle brain organoids contain cells that mostly have downregulated the genes for mitotic nuclear division. Panels I and J display enrichment plots showing that optic vesicle brain organoids contain cells with up-regulated gene expression for sensory perception and contain cells expressing genes for sensory perception of light stimuli. Panel K illustrates spontaneous action potentials in neurons, which were sometimes sensitive and sometimes resistant to Tetrodotoxin (TTX), which is a neurotoxin that selectively blocks sodium channels.

FIG. 4 illustrates optic vesicle brain organoids displaying non-neuronal components and optic tracts connected to brain organoids. Panel A shows 50-day-old optic vesicle 3D brain organoid-stained with F-actin marker Phalloidin. Panel B shows lenses neighboured by a single epithelial cell layer that is Keratin-3 positive. Panel C shows an ultrathin section of the optic vesicle region stained with toluidine blue staining showing a well-defined round lens in a cavity. Panel D shows a magnified view of the same lens. Panel E shows a schematic of optic vesicle brain organoids generation. Panel F shows organoid section primarily stained for PCP4. Panel G shows experimental scheme of CTB injection experiment.

FIG. 5 illustrates light sensitivity of optic vesicle brain organoids. Panel A shows A dose-dependent light response stimulated with three different light intensities. Panel B shows Isolating A-wave within the retinal signalling network. Panel C shows that photosensitivity of optic vesicle organoids can be desensitized by bright light exposure with the light intensity of 4600 Lux for 10 minutes.

FIG. 6 shows a section through an optic vesicle region stained with hematoxylin (panel A), surface rendered images of an optic vesicle 3D brain organoid stained with Phalloidin (panel B), adherent RPE cells dissociated from an optic vesicle from a monolayer RPE sheet displaying typical honeycomb shaped cells filled with pigments (panel C), and immunostaining through an optic vesicle.

FIG. 7 shows transmission EM image of a developing photoreceptor rod cell (panel A), and serial cross-sections of a developing rod outer segment (panel B).

FIG. 8 shows analysis of mRNA-seq data from early brain organoids and optic vesicle brain organoids (panel A), scatter plot of normalized counts from mRNA-seq data (panel B), affinity propagation clustering results (panel C), and leading-edge analysis of GSEA results for super-cluster 4 (panel D).

FIG. 9 shows expression profiling of optic vesicle brain organoids showing developmental eye signatures.

FIG. 10 shows expression profiling of optic vesicle brain organoids showing optic nerve formation signatures.

FIG. 11 shows a 50 day old optic vesicle 3D brain organoids injected with CTB-488 directly into an optic vesicle, and exposed to light for 10 min, cultured for 24 hours and then PFA fixed. Following tissue clearing, the specimens were imaged.

FIG. 12 illustrates photic stress experiments with isolated mouse retina. Panel A shows a mouse retina light response to 200.000 mlux light flashes of 500 ms duration. Panel B shows statistical summary of photic stress experiments with retinas from three different mice.

FIG. 13 shows optic vesicle organoids tested for the tropism of ZIKV to retinal progenitors (panels A-C), electron micrographs of ZIKV-AM infected optic vesicle organoids (panels D-G), and a bar diagram showing the ratio of infected vs. RAX positive cells.

### DETAILED DESCRIPTION OF THE INVENTION

Definitions

For the purpose of this invention, an “organoid” as used herein is a collection of organ-specific cell types that develops from stem cells or organ progenitors and can be generated through cell sorting and spatially restricted lineage commitment in a manner similar to in vivo. Organoid formation recapitulates both major processes of selective organizational steps during development: cell sorting out and spatially restricted lineage commitment. Thus, an organoid is a miniaturized and simplified version of an organ produced in vitro in three dimensions that show realistic micro-anatomy. Organoids have been described for various tissues such as intestinal.

“Brain organoids”, also termed cerebral organoids, contain several different brain regions within individual organoids. Cerebral organoids can reach sizes of up to a few millimetres when grown in a spinning bioreactor, which improves nutrient and oxygen exchange. This expansion allows the formation of a variety of brain regions, including retina, dorsal cortex, ventral forebrain, midbrain-hindbrain boundary, choroid plexus, and hippocampus. Optic vesicle organoids have been generated from mouse and human cells.

For the purpose of this invention, a “neuronal cell” or “neuron”, also known as a nerve cell, is an electrically excitable cell that communicates with other cells via specialized connections called synapses. It is the main component of nervous tissue. Neurons are typically classified into three types based on their function. Sensory neurons respond to stimuli such as light. Motor neurons control everything from muscle contractions to glandular output. Interneurons connect neurons to other neurons. A group of connected neurons is called a neural circuit. A typical neuron consists of a cell body (soma), dendrites, and a single axon. The axon and dendrites are filaments that extrude from it. Dendrites typically branch profusely and extend a few hundred micrometers from the soma. The axon leaves the soma at a swelling called the axon hillock. It branches but usually maintains a constant diameter. At the farthest tip of the axon's branches are axon terminals, where the neuron can transmit a signal across the synapse to another cell. Neurons may lack dendrites or have no axon. The term neurite is used to describe either a dendrite or an axon, particularly when the cell is undifferentiated. Most neurons receive signals via the dendrites and soma and send out signals down the axon. At the majority of synapses, signals cross from the axon of one neuron to a dendrite of another. However, synapses can connect an axon to another axon or a dendrite to another dendrite. The signalling process is partly electrical and partly chemical. Neurons are electrically excitable, due to maintenance of voltage gradients across their membranes. If the voltage changes by a large enough amounts over a short interval, the neuron generates an all-or-nothing electrochemical pulse called an action potential. This action potential travels rapidly along the axon, and activates synaptic connections as it reaches them. Synaptic signals may be excitatory or inhibitory, increasing or reducing the net voltage that reaches the soma.

Some non-limiting examples of neurons that are variably present in the invention of the present invention are:
Basket cells, interneurons that form a dense plexus of terminals around the soma of target cells, found in the cortex and cerebellum,
Betz cells, large motor neurons,
Lugaro cells, interneurons of the cerebellum,
Medium spiny neurons, most neurons in the corpus striatum,
Purkinje cells, huge neurons in the cerebellum, a type of Golgi I multipolar neuron,
Pyramidal cells, neurons with triangular soma, a type of Golgi I,
Renshaw cells, neurons with both ends linked to alpha motor neurons,
Unipolar brush cells, interneurons with unique dendrite ending in a brush-like tuft,
Granule cells, a type of Golgi II neuron,
Anterior horn cells, motoneurons located in the spinal cord, and
Spindle cells, interneurons that connect widely separated areas of the brain.

Neurons can, for example, be characterized by their expression of neurotransmitters. Thus, the neuronal cells of the present invention include:

Cholinergic neurons—acetylcholine. Acetylcholine is released from presynaptic neurons into the synaptic cleft. It acts as a ligand for both ligand-gated ion channels and metabotropic (GPCRs) muscarinic receptors. Nicotinic receptors are pentameric ligand-gated ion channels composed of alpha and beta subunits that bind nicotine. Ligand binding opens the channel causing influx of Na+ depolarization and increases the probability of presynaptic neurotransmitter release. Acetylcholine is synthesized from choline and acetyl coenzyme A.

GABAergic neurons—gamma aminobutyric acid. GABA is one of two neuroinhibitors in the central nervous system (CNS), along with glycine. GABA has a homologous function to ACh, gating anion channels that allow Cl− ions to enter the postsynaptic neuron. Cl− causes hyperpolarization within the neuron, decreasing the probability of an action potential firing as the voltage becomes more negative (for an action potential to fire, a positive voltage threshold must be reached). GABA is synthesized from glutamate neurotransmitters by the enzyme glutamate decarboxylase.

Glutamatergic neurons—glutamate. Glutamate is one of two primary excitatory amino acid neurotransmitters, along with aspartate. Glutamate receptors are one of four categories, three of which are ligand-gated ion channels and one of which is a G-protein coupled receptor (often referred to as GPCR).
AMPA and Kainate receptors function as cation channels permeable to Na+ cation channels mediating fast excitatory synaptic transmission.
NMDA receptors are another cation channel that is more permeable to Ca2+. The functions of NMDA receptors depend on glycine receptor binding as a co-agonist within the channel pore. NMDA receptors do not function without both ligands present.
Metabotropic receptors, GPCRs modulate synaptic transmission and postsynaptic excitability.
   Glutamate can cause excitotoxicity when blood flow to the brain is interrupted, resulting in brain damage. When blood flow is suppressed, glutamate is released from presynaptic neurons, causing greater NMDA and AMPA receptor activation than normal outside of stress conditions, leading to elevated Ca2+ and Na+ entering the postsynaptic neuron and cell damage. Glutamate is synthesized from the amino acid glutamine by the enzyme glutamate synthase.

Dopaminergic neurons—dopamine. Dopamine is a neurotransmitter that acts on D1 type (D1 and D5) Gs-coupled receptors, which increase cAMP and PKA, and D2 type (D2, D3, and D4) receptors, which activate Gi-coupled receptors that decrease cAMP and PKA. Dopamine is connected to mood and behavior and modulates both pre- and post-synaptic neurotransmission. Loss of dopamine neurons in the substantia nigra has been linked to Parkinson's disease. Dopamine is synthesized from the amino acid tyrosine. Tyrosine is catalyzed into levadopa (or L-DOPA) by tyrosine hydroxlase, and levadopa is then converted into dopamine by the aromatic amino acid decarboxylase.

Serotonergic neurons—serotonin. Serotonin (5-Hydroxytryptamine, 5-HT) can act as excitatory or inhibitory. Of its four 5-HT receptor classes, 3 are GPCR and 1 is a ligand-gated cation channel. Serotonin is synthesized from tryptophan by tryptophan hydroxylase, and then further by decarboxylase. A lack of 5-HT at postsynaptic neurons has been linked to depression. Drugs that block the presynaptic serotonin transporter are used for treatment, such as Prozac and Zoloft.

Neurons are generated by stem cells. A stem cell is, for example, a pluripotent stem cell, an embryonic stem cell, an adult stem cell, a neural stem cell or an embryonic germ cell. More preferably, a stem cell is selected from the group comprising an induced pluripotent stem cell (iPSC), an embryonic stem cell (ESC), a very small embryonic-like stem cell (VSEL), an amniotic fluid stem cell (AFSC), a marrow-isolated adult multilineage inducible cell (MIAMI), a multipotent adult precursor cell (MAPC) or an unrestricted somatic stem cell (USSC).

Neurogenesis largely ceases during adulthood in most areas of the brain. Moreover, neuronal cells, neuronal tissues and/or neuronal networks can be damaged or destroyed as a result of pathology or injury. Pathologies that are relevant for the present invention include, for example, age-related macular degeneration (AMD), retinitis pigmentosa (RP), Leber congenital amaurosis (LCA), Stargardt disease, choroideremia, Usher syndrome, X-linked retinoschisis (XLRS), achromatopsia (ACHM), and glaucoma. Examples of neurodegenerative diseases that are addressed by the present invention are Alzheimer's disease, Parkinson's disease, Huntington's disease, amyotrophic lateral sclerosis, frontotemporal dementia and the spinocerebellar ataxias. Moreover, neurodegenerative disorders like multiple sclerosis (including Clinically isolated syndrome (CIS), Relapsing-remitting MS (RRMS), Primary progressive MS (PPMS), and/or Secondary progressive MS (SPMS)), and chronic inflammatory demyelinating polyneuropathy represent a problem that the present invention seeks to overcome or to alleviate. This is because the prior art has not provided satisfactory approaches for providing and replenishing neuronal cells that are missing due to the lack of adult neurogenesis, pathology or injury.

Therefore, there is a need for sources of neuronal cells. Ideally, these neuronal cells can be used for medical, i.e. therapeutic or prophylactic purposes. The neuronal cells can, for example, be used for providing certain cell types, or for replenishing specific tissues in a patient. In this context or for other medical purposes, cells can be grafted or used for transplantation in a different setting. For instance, neuronal cells can be combined with non-neuronal cells. In addition, cells can be combined with artificial material such as polymers (such as biodegradable polymers like polyesters, polyamides, polyanhydrides, polycarbonates, polysaccharides, polylactic acid; proteins (such as fibronectin, laminin, collagen (type I, II, Ill, IV, V), hydroxyproline-rich proteins, integrins or fragments thereof, syndecans or fragments thereof, VEGF, HGF and BMP, metalloproteases, vitronectin), hyaluronic acid and its derivatives, cellulose and its derivatives such carboxymethyl cellulose or carboxyethyl cellulose or ions thereof, dextrane, polyethylene oxide, proteoglycans), fibers, ceramics, silicones, nanoparticles, pigments, metal ions (such as calcium, potassium, sodium ions), organic ions (such as pyruvate, oxalate, malonate, succinate, glutarate, adipate, citrate, amino acids, gluconic acid, glucuronic acid, deoxyribonucleic acid (DNA), ribonucleic acid (RNA), phosphorylated sugars), inorganic ions (such as bicarbonate, phosphate, borate).

In one embodiment, neuronal cells can be combined with non-neuronal cells and/or artificial material such as extracellular matrix (ECM) or ECM reagents (e.g. Matrigel™ from BD or BIOSILK 521 from BIOlaminaxxx). These reagents can be an ECM extract (such as from the Engelbreth-Holm-Swarm mouse tumor). Advantageously, they can be composed of laminin (e.g. 56%), collagen type IV (e.g. 31%), and enactin (e.g. 8%), and several growth factors, including EGF (0.7 ng/ml), PDGF (12 ng/ml), IGF-1 (16 ng/ml), and TGF-a (2.3 ng/ml) (14). In vivo and in vitro, formation of the extracellular matrix (ECM) by cells requires those cells to secrete ECM proteins. Assembly is achieved by following a strict hierarchical assembly pattern, which begins with the deposition of fibronectin filaments on the cell surface, a process known as fibrillogenesis. Unlike other ECM components that can self-polymerize under physiological conditions, fibronectin matrix assembly is a cell-dependent process. Cells continue to remodel the ECM by degradation and reassembly mechanisms, the dynamic nature of the ECM being particularly apparent during development, wound healing, and certain disease states. It is estimated that there are over 300 proteins comprising the mammalian ECM or “core matrisome” and this does not include the large number of ECM-associated proteins. Cells interact with the ECM through receptors such as integrins and syndecans, resulting in the transduction of multiple signals to regulate key cellular processes such as differentiation, proliferation, survival, and motility of cells. The ECM has also been shown to bind growth factors such as VEGF, HGF and BMPs, which are thought to create growth factor gradients that regulate pattern formation during development. Many of the ECM-regulated cell processes operate via reorganization of the actin and microtubule cytoskeletons.

In a preferred embodiment, cells are provided in combination with a synthetic basement membrane. The synthetic basement membrane may comprise a polyester membrane and/or parylene C. Preferably, the synthetic basement membrane may comprise a polyethylene terephthalate (PET) membrane, optionally coated in plasma-derived vitronectin. Preferably, the synthetic basement membrane is biodegradable.

In view of the above-identified need, the present invention provides a tissue isolated from a 3D brain organoid according to the present invention. The tissue may be a monolayer. In an embodiment, the tissue may comprise cells that are characterized by at least one of the following:
predominantly apical PEDF and basal BEST expression, and/or
PMEL17 expression, and/or
ZO1 expression, and/or
Absence of Ki67 expression, and/or
Expression of cellular retinaldehyde binding protein (CRALBP), and/or
Expression of MITF, and/or
Expression of OTX2, and/or
Secretion of PEDF, and/or
Cobblestone morphology, and/or
an ultrastructure comprising tight junctions marked by phalloidin, basal infoldings, apical microvilli, and melanin granules (Pigments).

The tissue may be provided as a patch. The patch may comprise at least one polyester membrane and/or parylene C. The patch may comprise polyethylene terephthalate (PET) membrane such as a PET membrane, optionally coated in plasma-derived vitronectin.

The tissue may be an (artificial) retinal pigment epithelium (RPE). The tissue may be used in the treatment of diseases associated with visual impairment such as age-related macular degeneration (AMD), retinitis pigmentosa (RP), Leber congenital amaurosis (LCA), Stargardt disease, choroideremia, Usher syndrome, X-linked retinoschisis (XLRS), achromatopsia (ACHM), and glaucoma.

In view of the above-identified need, the present invention additionally provides cells and/or tissues and/or combinations of cells and synthetic materials for treating neurological disorders, including but not limited to, Charcot-Marie-Tooth disease (CMT), Alzheimer's disease (AD), Parkinson's disease (PD), and Myasthenia gravis.

Charcot-Marie-Tooth disease (CMT) is a heterogeneous inherited disorder of nerves (neuropathy) that is characterized by loss of muscle tissue and touch sensation, predominantly in the feet and legs extending to the hands and arms in advanced stages. Presently incurable, this disease is one of the most common inherited neurological disorders, with 36 in 100,000 affected.

Alzheimer's disease (AD), also known simply as Alzheimer's, is a neurodegenerative disease characterized by progressive cognitive deterioration, together with declining activities of daily living and neuropsychiatric symptoms or behavioural changes. The most striking early symptom is loss of short-term memory (amnesia), which usually manifests as minor forgetfulness that becomes steadily more pronounced with illness progression, with relative preservation of older memories. As the disorder progresses, cognitive (intellectual) impairment extends to the domains of language (aphasia), skilled movements (apraxia), and recognition (agnosia), and functions such as decision-making and planning become impaired.

Parkinson's disease (PD), also known as Parkinson disease, is a degenerative disorder of the central nervous system that often impairs motor skills and speech. Parkinson's disease belongs to a group of conditions called movement disorders. It is characterized by muscle rigidity, tremor, a slowing of physical movement (bradykinesia), and in extreme cases, a loss of physical movement (akinesia). The primary symptoms are the results of decreased stimulation of the motor cortex by the basal ganglia, normally caused by the insufficient formation and action of dopamine, which is produced in the dopaminergic neurons of the brain. Secondary symptoms may include high-level cognitive dysfunction and subtle language problems. PD is both chronic and progressive.

Myasthenia gravis is a neuromuscular disease leading to fluctuating muscle weakness and fatigability during simple activities. Circulating antibodies that block acetylcholine receptors at the post-synaptic neuromuscular junction, inhibiting the stimulative effect of the neurotransmitter acetylcholine, typically causes weakness. Myasthenia is treated with immunosuppressants, cholinesterase inhibitors and, in selected cases, thymectomy.

In view of the above-identified need, the present invention additionally provides cells and/or tissues and/or combinations of cells and synthetic materials for treating demyelinating diseases. Demyelination is the act of demyelinating, or the loss of the myelin sheath insulating the nerves. When myelin degrades, conduction of signals along the nerve can be impaired or lost, and the nerve eventually withers. This leads to certain neurodegenerative disorders like multiple sclerosis (including Clinically isolated syndrome (CIS), Relapsing-remitting MS (RRMS), Primary progressive MS (PPMS), and/or Secondary progressive MS (SPMS)), and chronic inflammatory demyelinating polyneuropathy.

“Non-neuronal cells” as used herein include surface ectodermal cells, glia, epithelial cells, pericytes, and endothelia, lens cells, cornea cells and the like. In a preferred embodiment, non-neuronal cells include cornea cells and/or lens cells. Preferably, a lens expresses genes selected from the group of lens markers consisting of CRYAB, CRYBB3, and/or the cornea expresses genes selected from the group of cornea markers consisting of OPTC; KRT3.

“Ectoderm” as used herein is one of the three primary germ layers in the very early embryo. The other two layers are the mesoderm (middle layer) and endoderm (most proximal layer), with the ectoderm as the most exterior (or distal) layer. Generally speaking, the ectoderm differentiates to form the nervous system (spine, peripheral nerves and brain), tooth enamel and the epidermis (the outer part of integument). It also forms the lining of mouth, anus, nostrils, sweat glands, hair and nails. In vertebrates, the ectoderm has three parts: external ectoderm (also known as surface ectoderm), the neural crest, and neural tube. The latter two are known as neuroectoderm. Ectodermal cells originate from a germline lineage, i.e. ectoderm. This lineage may be present in a cellular organization process such embryology. Embryology may proceed in vivo or in vitro. In vitro, stem cells may be induced to produce ectodermal cells. A stem cell is, for example, a pluripotent stem cell, an embryonic stem cell, an adult stem cell, a neural stem cell or an embryonic germ cell. More preferably, a stem cell is selected from the group comprising an induced pluripotent stem cell (iPSC), an embryonic stem cell (ESC), a very small embryonic-like stem cell (VSEL), an amniotic fluid stem cell (AFSC), a marrow-isolated adult multilineage inducible cell (MIAMI), a multipotent adult precursor cell (MAPC) or an unrestricted somatic stem cell (USSC). Moreover, cells that are ectodermal cells are cells that can be characterized as being ectodermal based on their expression pattern, their morphology and/or other cytological markers.

There is a need for sources of ectodermal cell in order to be able to study physiological processes as illustrated in the examples and/or in order to be able to replenish or transplant tissues such as lens or cornea tissues.

In general terms, the “surface ectoderm” (or external ectoderm) as used herein forms the following structures:
Skin (only epidermis; dermis is derived from mesoderm) (along with glands, hair, and nails),
Epithelium of the mouth and nasal cavity and glands of the mouth and nasal cavity,
Tooth enamel (as a side note, dentin and dental pulp are formed from ectomesenchyme which is derived from ectoderm (specifically neural crest cells and travels with mesenchmyal cells),
Epithelium of anterior pituitary,
Lens, cornea, lacrimal gland, tarsal glands and the conjunctiva of the eye,
Apical ectodermal ridge inducing development of the limb buds of the embryo, and/or
Sensory receptors in epidermis.

In the present invention, the term surface ectoderm preferably relates to the lens and/or the cornea.

In general terms, “neuroectoderm” (or neural ectoderm or neural tube epithelium) as used herein consists of cells derived from ectoderm. Formation of the neuroectoderm is first step in the development of the nervous system. The neuroectoderm receives bone morphogenetic protein-inhibiting signals from proteins such as noggin, which leads to the development of the nervous system from this tissue. Histologically, these cells are classified as pseudostratified columnar cells. After recruitment from the ectoderm, the neuroectoderm undergoes three stages of development: transformation into the neural plate, transformation into the neural groove (with associated neural folds), and transformation into the neural tube. After formation of the tube, the brain forms into three sections; the hindbrain, the midbrain, and the forebrain.

In an embodiment, neuroectoderm may include one or more of the following:
neural crest,
pigment cells in the skin,
ganglia of the autonomic nervous system,
dorsal root ganglia,
facial cartilage,
aorticopulmonary septum of the developing heart and lungs,
ciliary body of the eye,
adrenal medulla,
parafollicular cells in the thyroid,
neural tube,
brain (rhombencephalon, mesencephalon and prosencephalon),
spinal cord and motor neurons,
retina, and
posterior pituitary.

In the present invention, the term neuroectoderm preferably relates to brain and/or retinal tissue.

EP2497825 A1 discloses methods that can be used for the purposes of the present invention. According to EP2497825 A1, neuroectodermal cells can be generated by a method (incorporated herein by reference) comprising: culturing pluripotent cells for about 2 to about 6 days, wherein the expression and/or activity of one or more proteins selected from the group consisting of fibroblast growth factor receptor (FGFR), son of sevenless (SOS), growth factor receptor-bound protein 2 (GRB2), rat sarcoma protein (RAS), rat fibrosarcoma (RAF), mitogen-activated protein kinase kinase (MEK) and mitogen-activated protein kinase (MAPK) is inhibited. The human pluripotent cells are preferably cultured for at least about 4 days. Additionally, the nodal/activin/tumour growth factor beta/small mothers against decapentaplegic 2/3 (Nodal/activin/TGFbeta/SMAD2/3) signalling pathway; and/or the bone morphogenic protein/growth differentiation factor/small mothers against decapentaplegic 1/5/8 (BMP/GDF/SMAD1/5/8) signalling pathway may be inhibited. The nodal/activin/TGFbeta/SMAD2/3 signalling pathway may be inhibited by one or more compounds selected from the group consisting of 4-[4-(3,4-Methylenedioxyphenyl)-5-(2-pyridyl)-1H-imidazol-2-yl]-benzamide hydrate (SB431542), 3-(6-Methylpyridin-2-yl)-1-phenylthiocarbamoyl-4-quinolin-4-ylpyrazole (A-83-01) and follistatin; and/or the BMP/GDF/SMAD1/5/8 signalling pathway may be inhibited by one or more compounds selected from the group consisting of 6-[4-[2-(1-Piperidinyl)ethoxy]phenyl]-3-(4-pyridinyl)-pyrazolo[1,5-a]pyrimidine dihydrochloride (dorsomorphin), noggin and gremlin. Moreover, the BMP/GDF/SMAD1/5/8 signalling pathway could be additionally inhibited. Further, the expression and/or activity of FGFR, MEK and/or MAPK may be inhibited.

The activity of MEK may be inhibited by one or more of the compounds selected from the group consisting of 2-(2-Chloro-4-iodo-phenylamino)-N-cyclopropylmethoxy-3,4-difluoro-benzamide (PD184352), N-[(2R)-2,3-dihydroxypropoxy]-3,4-difluoro-2-[(2-fluoro-4-iodophenyl) amino]-benzamide (PD0325901), 2′-Amino-3′-methoxyflavone (PD98059) and 1,4-diamino-2,3-dicyano-1,4-bis (2-aminophenylthio)butadiene (U0126); and/or wherein the activity of FGFR is inhibited by 2-[(1,2-Dihydro-2-oxo-3H-indol-3-ylidene)methyl]-4-methyl-1H-pyrrole-3-propanoic acid (SU5402) and/or N-[2-[[4-(Diethylamino)butyl]amino]-6-(3,5-dimethoxyphenyl)pyrido[2,3-d]pyrimidin-7-yl]-N′-(1,1-dimethylethyl)urea (PD173074).

According to EP2497825 A1, neurons can be generated by a method (incorporated herein) comprising the steps of generating neuroectodermal cells and culturing the neuroectoderm cells for at least about 2 further days, preferably for at least about 4 days.

“Neuroectodermal cells” as used herein originate from a germline lineage, i.e. ectoderm which gives rise to the neuroectoderm. This lineage may be present in a cellular self-organization process such embryology. Embryology may proceed in vivo or in vitro. In vitro, stem cells may be used to produce neuroectodermal cells. A stem cell is, for example, a pluripotent stem cell, an embryonic stem cell, an adult stem cell, a neural stem cell or an embryonic germ cell. More preferably, a stem cell is selected from the group comprising an induced pluripotent stem cell (iPSC), an embryonic stem cell (ESC), a very small embryonic-like stem cell (VSEL), an amniotic fluid stem cell (AFSC), a marrow-isolated adult multilineage inducible cell (MIAMI), a multipotent adult precursor cell (MAPC) or an unrestricted somatic stem cell (USSC). Moreover, cells that are neuroectodermal cells are cells that can be characterized as being neuroectodermal based on their expression pattern, their morphology and/or other cytological markers.

“Surface ectodermal cells” as used herein originate from a germline lineage, i.e. ectoderm which gives rise to the ectoderm and then the surface ectoderm. This lineage may be present in a cellular self-organization process such embryology. Embryology may proceed in vivo or in vitro. In vitro, stem cells may be used to produce surface ectodermal cells. A stem cell is, for example, a pluripotent stem cell, an embryonic stem cell, an adult stem cell, a neural stem cell or an embryonic germ cell. More preferably, a stem cell is selected from the group comprising an induced pluripotent stem cell (iPSC), an embryonic stem cell (ESC), a very small embryonic-like stem cell (VSEL), an amniotic fluid stem cell (AFSC), a marrow-isolated adult multilineage inducible cell (MIAMI), a multipotent adult precursor cell (MAPC) or an unrestricted somatic stem cell (USSC). Cells that represent surface ectodermal cells are cells that can be characterized as being surface ectodermal based on their expression pattern, their morphology and/or other cytological markers.

The term “vascularization” relates to the formation of blood vessels and capillaries in living tissue. The term “vasculature” relates to blood vessels or to an arrangement of blood vessels in an organ or part thereof.

Cells related to blood circulation comprise blood cells. A “blood cell”, also called a hematopoietic cell, hemocyte, or hematocyte, as used herein is a cell produced through hematopoiesis in the bone marrow and found mainly in the blood or cord blood. Major types of blood cells include red blood cells (erythrocytes), white blood cells (leukocytes), and platelets (thrombocytes).

Various cell markers can be used to ascertain the absence of blood vessels, vasculature and cells related to blood circulation.

These markers and their main markers and functions are listed below:

CD4 is a glycoprotein found on the surface of immune cells such as T helper cells, monocytes, macrophages, and dendritic cells. CD4+ T helper cells are white blood cells that are an essential part of the human immune system. CD4 interacts with the β2-domain of MHC class II molecules through its D1 domain. T cells displaying CD4 molecules (and not CD8) on their surface, therefore, are specific for antigens presented by MHC II and not by MHC class I.

CD8 is a transmembrane glycoprotein that serves as a co-receptor for the T cell receptor (TCR). The CD8 co-receptor is predominantly expressed on the surface of cytotoxic T cells, but can also be found on natural killer cells, cortical thymocytes, and dendritic cells. Like the TCR, CD8 binds to a major histocompatibility complex (MHC) molecule, but is specific for the class I MHC protein.

CD25 is a type I transmembrane protein present on activated T cells, activated B cells, some thymocytes, myeloid precursors, and oligodendrocytes that associates with CD122 to form a heterodimer that can act as a high-affinity receptor for IL-2; expressed in most B-cell neoplasms, some acute nonlymphocytic leukemias, and neuroblastomas.

CD33 is a marker of unknown function found on immature myeloid cells, including acute myeloid leukemia blasts and mature monocytes.

CD40 is a costimulatory protein found on antigen presenting cells.

CD43 is the major sialic acid rich protein on the surface of lymphocytes. It is a transmembrane protein with functions in T-cell immunity.

CD45 is a leucocyte common antigen, a type I transmembrane protein present on all hemopoietic cells except erythrocytes that assists in cell activation; expressed in lymphomas, B-cell chronic lymphocytic leukemia, hairy cell leukemia, and acute nonlymphocytic leukemia.

CD48 is a human protein encoded by the CD48 gene. It is a universal cell membrane molecule present on all leukocytes.

CD56 140 kD isoform of NCAM (neural cell adhesion molecule), a marker for natural killer cells and some T-lymphocytes.

CD57 is expressed by Natural Killer Cells subsets of T cells, B Cells, and Monocytes. Represents a carbohydrate epitope that contains a sulfoglucuronyl residue.

CD62L (L-selectin) is a cell adhesion molecule found on leukocytes.

CD79A, also known as B-cell antigen receptor complex-associated protein alpha chain and MB-1 membrane glycoprotein, is a protein that in humans is encoded by the CD79A gene. Together with CD79B, forms a dimer associated with the formation of the B-cell antigen receptor (BCR), enabling a cell to respond to the presence of antigens on its surface.

CD79B, also known as B-cell antigen receptor complex-associated protein beta chain, is a protein that in humans is encoded by the CD79B gene. Together with CD79A, forms a dimer associated with the formation of the B-cell antigen receptor (BCR), enabling a cell to respond to the presence of antigens on its surface. Roche is developing an antibody drug conjugate (RG7596) targeting CD76b in certain types of Non-Hogdkin Lymphoma. Macrogenics has started pre-clinical studies into a drug (MGD010) targeting CD79b and CD32b.

CD115, also known as colony stimulating factor 1 receptor (CSF1R) and/or as macrophage colony-stimulating factor receptor (M-CSFR), is a receptor, known to be expressed on monocytes and macrophages, for a cytokine called colony stimulating factor 1 (CSF1) and also interleukin 34 (IL34).

CD160 expression is tightly associated with peripheral blood NK cells and CD8 T lymphocytes with cytolytic effector activity. In tissues, CD160 is expressed on all intestinal intraepithelial lymphocytes.

CD163 is glycoprotein endocytic scavenger receptor for haptoglobin-hemoglobin complexes. Found specifically on monocytes/macrophages and some dendritic cells.

CD300A is present on monocytes, neutrophils, and some T and B lymphocytes.

The following is a non-exhaustive list of B cell markers:
CD19: transmembrane protein that, in humans, is expressed in all B lineage cells, except for plasma cells, and in follicular dendritic cells,
CD20: expressed on the surface of all B-cells beginning at the pro-B phase (CD45R+, CD117+) and progressively increasing in concentration until maturity,
CD27: a memory B-cell marker, and
CD268: B-cell activating factor (BAFF) is a type III transmembrane protein containing a single extracellular phenylalanine-rich domain. It is thought that this receptor is the principal receptor required for BAFF-mediated mature B-cell survival.

Exemplary T Cell Markers:
CD3: T cell co-receptor that helps to activate both the cytotoxic T cell (CD8+ naive T cells) and also T helper cells (CD4+ naive T cells). It consists of a protein complex and is composed of four distinct chains. In mammals, the complex contains a CD3γ chain, a CD3δ chain, and two CD3ε chains.

Exemplary Macrophage
CD14: a membrane protein found on macrophages which binds to bacterial lipopolysaccharide.

Exemplary Natural Killer Cells:
CD96 is a transmembrane glycoprotein that has three extracellular immunoglobulin-like domains and is expressed by all resting human and mouse NK cells.

Blood cell markers can be analysed by common techniques such as immunohistochemistry, Magnetic-Activated Cell Sorting (MACS) and Fluorescence-Activated Cell Sorting (FACS).

During embryonic development of the eye, the outer wall of the bulb of the optic vesicles becomes thickened and invaginated, and the bulb is thus converted into a vesicle, the so-called “optic vesicle” (or ophthalmic vesicle), consisting of two strata of cells. These two strata are continuous with each other at the vesicle margin, which ultimately overlaps the front of the lens and reaches as far forward as the future aperture of the pupil.

The optic vesicle is part of the diencephalon and gives rise to the retina of the eye.

In a preferred embodiment of the invention, the first cells comprise retinal cells. The “retina” is a light-sensitive layer of tissue. The neural retina consists of several layers of neurons interconnected by synapses, and is supported by an outer layer of pigmented epithelial cells. The primary light-sensing cells in the retina are the photoreceptor cells, which are of two types: rods and cones. Rods function mainly in dim light and provide black-and-white vision. Cones function in well-lit conditions and are responsible for the perception of colour, as well as high-acuity vision used for tasks such as reading. A third type of light-sensing cell, the photosensitive ganglion cell, is important for entrainment of circadian rhythms and reflexive responses such as the pupillary light reflex.

Light striking the retina initiates a cascade of chemical and electrical events that ultimately trigger nerve impulses that are sent to various visual centres of the brain through the fibres of the optic nerve. Neural signals from the rods and cones undergo processing by other neurons, whose output takes the form of action potentials in retinal ganglion cells whose axons form the optic nerve. Several important features of visual perception can be traced to the retinal encoding and processing of light by these cerebral optic vesicles.

“Rhodopsin (RHO)” is a light-sensitive receptor protein provided by the present invention, e.g. for being expressed in the (optic vesicle) 3D brain organoid according to the invention. It is involved in visual phototransduction. Rhodopsin is a biological pigment found in the rods of the retina and is a G-protein-coupled receptor (GPCR). It belongs to opsins. Rhodopsin is extremely sensitive to light, and thus enables vision in low-light conditions. When rhodopsin is exposed to light, it immediately photobleaches. In humans, it is regenerated fully in about 30 minutes, after which rods are more sensitive. Rhodopsin consists of two components, a protein molecule also called scotopsin and a covalently-bound cofactor called retinal. Scotopsin is an opsin, a light-sensitive G protein coupled receptor that embeds in the lipid bilayer of cell membranes using seven protein transmembrane domains. These domains form a pocket where the photoreactive chromophore, retinal, lies horizontally to the cell membrane, linked to a lysine residue in the seventh transmembrane domain of the protein. Thousands of rhodopsin molecules are found in each outer segment disc of the host rod cell. Retinal is produced in the retina from vitamin A, from dietary beta-carotene. Isomerization of 11-cis-retinal into all-trans-retinal by light sets off a series of conformational changes (‘bleaching’) in the opsin, eventually leading it to a form called metarhodopsin II (Meta II), which activates an associated G protein, transducin, to trigger a cyclic guanosine monophosphate (cGMP) second messenger cascade.

“Opsins (OPN)” are provided by the present invention, e.g. for being expressed in the (optic vesicle) 3D brain organoid according to the invention. They are a group of proteins, made light-sensitive, via the chromophore retinal (or a variant) found in photoreceptor cells of the retina. Five classical groups of opsins are involved in vision, mediating the conversion of a photon of light into an electrochemical signal, the first step in the visual transduction cascade. Another opsin found in the mammalian retina, melanopsin, is involved in circadian rhythms and pupillary reflex but not in vision. Opsins can be classified in several ways, including function (vision, phototaxis, photoperiodism, etc.), type of chromophore (retinal, flavine, bilin), molecular structure (tertiary, quaternary), signal output (phosphorylation, reduction, oxidation). There are two groups of protein termed opsins. Type I opsins are employed by prokaryotes and by some algae (as a component of channelrhodopsins) and fungi, whereas animals use type II opsins. These latter are within the scope of the present invention.

“Cone-rod homeobox protein (CRX)” is a transcription factor provided by the present invention, e.g. for being expressed in the (optic vesicle) 3D brain organoid according to the invention. It binds and transactivates the sequence 5′-TAATC[CA]-3′ which is found upstream of several photoreceptor-specific genes, including the opsin genes. It acts synergistically with other transcription factors, such as NRL, RORB and RAX, to regulate photoreceptor cell-specific gene transcription. It is essential for the maintenance of mammalian photoreceptors.Recoverin (RCVRN).

Recoverin is a 23 kilodalton (kDa) neuronal calcium-binding protein that is primarily detected in the photoreceptor cells of the eye. It plays a key role in the inhibition of rhodopsin kinase, a molecule which regulates the phosphorylation of rhodopsin. A reduction in this inhibition helps regulate sensory adaptation in the retina, since the light-dependent channel closure in photoreceptors causes calcium levels to decrease, which relieves the inhibition of rhodopsin kinase by calcium-bound recoverin, leading to a more rapid inactivation of metarhodopsin II (activated form of rhodopsin).

“Neural retina-specific leucine zipper protein (NRL)” is a protein that in humans is encoded by the NRL gene. This gene encodes a basic motif-leucine zipper transcription factor of the Maf subfamily. The encoded protein is conserved among vertebrates and is a critical intrinsic regulator of photoceptor development and function. Mutations in this gene have been associated with retinitis pigmentosa and retinal degenerative diseases. NRL is provided by the present invention, e.g. for being expressed in the (optic vesicle) 3D brain organoid according to the invention.

The BHLHE22 gene encodes a protein that belongs to the basic helix-loop-helix (bHLH) family of transcription factors that regulate cell fate determination, proliferation, and differentiation. A similar protein in mouse is required for the development of the dorsal cochlear nuclei, and is thought to play a role in in the differentiation of neurons involved in sensory input. The mouse protein also functions in retinogenesis. BHLHE22 is provided by the present invention, e.g. for being expressed in the (optic vesicle) 3D brain organoid according to the invention.

The PRDM8 gene encodes a protein that belongs to a conserved family of histone methyltransferases that predominantly act as negative regulators of transcription. The encoded protein contains an N-terminal Su(var)3-9, Enhancer-of-zeste, and Trithorax (SET) domain and a double zinc-finger domain. Knockout of this gene in mouse results in mistargeting by neurons of the dorsal telencephalon, abnormal itch-like behavior, and impaired differentiation of rod bipolar cells. In humans, the protein has been shown to interact with the phosphatase laforin and the ubiquitin ligase malin, which regulate glycogen construction in the cytoplasm. Alternative splicing results in multiple transcript variants. PRDM8 is provided by the present invention, e.g. for being expressed in the (optic vesicle) 3D brain organoid according to the invention.

Protein kinase C alpha (PKCα) is an enzyme that in humans is encoded by the PRKCA gene. PKCα is provided by the present invention, e.g. for being expressed in the (optic vesicle) 3D brain organoid according to the invention.

Protein kinase C (PKC) is a family of serine- and threonine-specific protein kinases that can be activated by calcium and the second messenger diacylglycerol. PKC family members phosphorylate a wide variety of protein targets and are known to be involved in diverse cellular signaling pathways. PKC family members also serve as major receptors for phorbol esters, a class of tumor promoters. Each member of the PKC family has a specific expression profile and is believed to play a distinct role in cells. The protein encoded by this gene is one of the PKC family members. This kinase has been reported to play roles in many different cellular processes, such as cell adhesion, cell transformation, cell cycle checkpoint, and cell volume control. Knockout studies in mice suggest that this kinase may be a fundamental regulator of cardiac contractility and Ca2+ handling in myocytes.

Protein kinase C-alpha (PKC-α) is a specific member of the protein kinase family. These enzymes are characterized by their ability to add a phosphate group to other proteins, thus changing their function. PKC-α has been widely studied in the tissues of many organisms including drosophila, xenopus, cow, dog, chicken, human, monkey, mouse, pig, and rabbit. Many studies are currently being conducted investigating the structure, function, and regulation of this enzyme. The most recent investigations concerning this enzyme include its general regulation, hepatic function, and cardiac function.

The ATOH7 gene encodes a member of the basic helix-loop-helix family of transcription factors, with similarity to Drosophila atonal gene that controls photoreceptor development. Studies in mice suggest that this gene plays a central role in retinal ganglion cell and optic nerve formation. Mutations in this gene are associated with nonsyndromic congenital retinal nonattachment. ATOH7 is provided by the present invention, e.g. for being expressed in the (optic vesicle) 3D brain organoid according to the invention.

Homeobox protein DLX-2 is a protein that in humans is encoded by the DI X2 gene. DLX2 is provided by the present invention, e.g. for being expressed in the (optic vesicle) 3D brain organoid according to the invention.

Many vertebrate homeo box-containing genes have been identified on the basis of their sequence similarity with Drosophila developmental genes. Members of the Dlx gene family contain a homeobox that is related to that of Distal-less (DII), a gene expressed in the head and limbs of the developing fruit fly. The Distal-less (Dlx) family of genes comprises at least 6 different members, DLX1-DLX6. The DLX proteins are postulated to play a role in forebrain and craniofacial development. This gene is located in a tail-to-tail configuration with another member of the gene family on the long arm of chromosome 2.

Syntaxin-1A is a protein that in humans is encoded by the STX1A gene. STX1A is provided by the present invention, e.g. for being expressed in the (optic vesicle) 3D brain organoid according to the invention.

Synaptic vesicles store neurotransmitters that are released during calcium-regulated exocytosis. The specificity of neurotransmitter release requires the localization of both synaptic vesicles and calcium channels to the presynaptic active zone. Syntaxins function in this vesicle fusion process.

Syntaxin-1A is a member of the syntaxin superfamily. Syntaxins are nervous system-specific proteins implicated in the docking of synaptic vesicles with the presynaptic plasma membrane. Syntaxins possess a single C-terminal transmembrane domain, a SNARE [Soluble NSF (N-ethylmaleimide-sensitive fusion protein)-Attachment protein REceptor] domain (known as H3), and an N-terminal regulatory domain (Habc). Syntaxins bind synaptotagmin in a calcium-dependent fashion and interact with voltage dependent calcium and potassium channels via the C-terminal H3 domain. Syntaxin-1A is a key protein in ion channel regulation and synaptic exocytosis.

“Calbindins” are three different calcium-binding proteins: calbindin, calretinin and S100G. They were originally described as vitamin D-dependent calcium-binding proteins in the intestine and kidney in the chick and mammals. They are now classified in different subfamilies as they differ in the number of Ca²⁺ binding EF hands. CALB2 is provided by the present invention, e.g. for being expressed in the (optic vesicle) 3D brain organoid according to the invention.

Calretinin, also known as calbindin 2, is a 29 kDa protein with 58% homology to calbindin 1 and principally found in nervous tissues. It is encoded in humans by the CALB2 gene and was formerly known as calbindin-D29k.

Retinal pigment epithelium-specific 65 kDa protein, also known as “retinoid isomerohydrolase”, is an enzyme of the vertebrate visual cycle that is encoded in humans by the RPE65 gene. RPE65 is expressed in the retinal pigment epithelium (RPE, a layer of epithelial cells that nourish the photoreceptor cells) and is responsible for the conversion of all-trans-retinyl esters to 11-cis-retinol during phototransduction. 11-cis-retinol is then used in visual pigment regeneration in photoreceptor cells. RPE65 belongs to the carotenoid oxygenase family of enzymes. RPE65 is provided by the present invention, e.g. for being expressed in the (optic vesicle) 3D brain organoid according to the invention.

Retinaldehyde-binding protein 1 (RLBP1) also known as “cellular retinaldehyde-binding protein (CRALBP)” is a 36-kD water-soluble protein that in humans is encoded by the RLBP1 gene. Cellular retinol binding protein (CRBP) was first discovered in 1973 from lung tissues by Bashor et al. There have been three cellular retinol binding protein categories discovered; Cellular retinol-binding protein, cellular retinoic acid-binding protein and cellular retinaldehyde-binding protein (CRALBP). CRALBP is provided by the present invention, e.g. for being expressed in the (optic vesicle) 3D brain organoid according to the invention. CRALBP is not just found in retina and retinal pigment epithelial cells, but also expressed in other cell types. It is majorly found in the iris, cornea, ciliary epithelium, Muller cells, the pineal gland and oligodendrocytes of the optic nerve and brain. This protein is also found in other tissues than the aforementioned ones, however its function in cells not related to the eyes are not yet known.

“ADP Ribosylation Factor Like GTPase” is a cilium-specific protein required to control the microtubule-based, ciliary axoneme structure. It may act by maintaining the association between IFT subcomplexes A and B. it binds GTP but is not able to hydrolyze it; the GTPase activity remains unclear. It is required to pattern the neural tube. It is Involved in cerebral cortex development: required for the initial formation of a polarized radial glial scaffold, the first step in the construction of the cerebral cortex, by regulating ciliary signaling. It regulates the migration and placement of postmitotic interneurons in the developing cerebral cortex. Arl13B is provided by the present invention, e.g. for being expressed in the (optic vesicle) 3D brain organoid according to the invention.

“Actin” is a family of globular multi-functional proteins that form microfilaments. It is found in essentially all eukaryotic cells, where it may be present at a concentration of over 100 pM; its mass is roughly 42-kDa, with a diameter of 4 to 7 nm. Actin is provided by the present invention, e.g. for being expressed in the (optic vesicle) 3D brain organoid according to the invention. An actin protein is the monomeric subunit of two types of filaments in cells: microfilaments, one of the three major components of the cytoskeleton, and thin filaments, part of the contractile apparatus in muscle cells. It can be present as either a free monomer called G-actin (globular) or as part of a linear polymer microfilament called F-actin (filamentous), both of which are essential for such important cellular functions as the mobility and contraction of cells during cell division.

“Bestrophin-1 (Best1)” is a protein that, in humans, is encoded by the BEST1 gene (RPD ID—5T5N/4RDQ). BEST1 is provided by the present invention, e.g. for being expressed in the (optic vesicle) 3D brain organoid according to the invention. The bestrophin family of proteins comprises four evolutionary related genes (BEST1, BEST2, BEST3, and BEST4) that code for integral membrane proteins. This family was first identified in humans by linking a BEST1 mutation with Best vitelliform macular dystrophy (BVMD). Mutations in the BEST1 gene have been identified as the primary cause for at least five different degenerative retinal diseases. In humans, bestrophins function as calcium-activated anion channels, each of which has a unique tissue distribution throughout the body. Specifically, the BEST1 gene on chromosome 11q13 encodes the Bestrophin-1 protein in humans whose expression is highest in the retina.

“Synapsin I”, is the collective name for Synapsin Ia and Synapsin Ib, two nearly identical phosphoproteins that in humans are encoded by the SYN1 gene. In its phosphorylated form, Synapsin I may also be referred to as phosphosynaspin I. Synapsin I is the first of the proteins in the synapsin family of phosphoproteins in the synaptic vesicles present in the central and peripheral nervous systems. Synapsin la and lb are close in length and almost the same in make up, however, Synapsin Ib stops short of the last segment of the C-terminal in the amino acid sequence found in Synapsin Ia. SYN1 is provided by the present invention, e.g. for being expressed in the (optic vesicle) 3D brain organoid according to the invention.

“Myelin basic protein (MBP)” is a protein believed to be important in the process of myelination of nerves in the nervous system. The myelin sheath is a multi-layered membrane, unique to the nervous system, that functions as an insulator to greatly increase the velocity of axonal impulse conduction. MBP maintains the correct structure of myelin, interacting with the lipids in the myelin membrane. MBP is provided by the present invention, e.g. for being expressed in the (optic vesicle) 3D brain organoid according to the invention. MBP knockout mice called shiverer mice were developed and characterized in the early 1980s. Shiverer mice exhibit decreased amounts of CNS myelination and a progressive disorder characterized by tremors, seizures, and early death. The human gene for MBP is on chromosome 18; the protein localizes to the CNS and to various cells of the hematopoietic lineage. The pool of MBP in the central nervous system is very diverse, with several splice variants being expressed and a large number of post-translational modifications on the protein, which include phosphorylation, methylation, deamidation, and citrullination. These forms differ by the presence or the absence of short (10 to 20 residues) peptides in various internal locations in the sequence. In general, the major form of MBP is a protein of about 18.5 Kd (170 residues).

“Class III β-tubulin (TUJ1)”, otherwise known as βIII-tubulin (β3-tubulin) or β-tubulin III, is a microtubule element of the tubulin family found almost exclusively in neurons, and in testis cells. In humans, it is encoded by the TUBB3 gene. TUJ1 is provided by the present invention, e.g. for being expressed in the (optic vesicle) 3D brain organoid according to the invention. It is possible to use monoclonal antibodies and immunohistochemistry to identify neurons in samples of brain tissue, separating neurons from glial cells, which do not express Class III β-tubulin.

“Purkinje cell protein 4” is a protein that in humans is encoded by the PCP4 gene. Also known as PEP-19, PCP4 is a 7.6 kDa protein with an IQ-motif that binds to calmodulin (CaM). PCP4 is abundant in Purkinje cells of the cerebellum, and plays an important role in synaptic plasticity. PCP4 is provided by the present invention, e.g. for being expressed in the (optic vesicle) 3D brain organoid according to the invention.

“Laminins” are high-molecular weight (˜400 to ˜900 kDa) proteins of the extracellular matrix. They are a major component of the basal lamina (one of the layers of the basement membrane), a protein network foundation for most cells and organs. The laminins are an important and biologically active part of the basal lamina, influencing cell differentiation, migration, and adhesion. Laminin is provided by the present invention, e.g. for being expressed in the (optic vesicle) 3D brain organoid according to the invention. Laminins are heterotrimeric proteins that contain an α-chain, a β-chain, and a γ-chain, found in five, four, and three genetic variants, respectively. The laminin molecules are named according to their chain composition. Thus, laminin-511 contains α5, β1, and γ1 chains. Fourteen other chain combinations have been identified in vivo. The trimeric proteins intersect to form a cross-like structure that can bind to other cell membrane and extracellular matrix molecules. The three shorter arms are particularly good at binding to other laminin molecules, which allows them to form sheets. The long arm is capable of binding to cells, which helps anchor organized tissue cells to the membrane. The laminin family of glycoproteins are an integral part of the structural scaffolding in almost every tissue of an organism. They are secreted and incorporated into cell-associated extracellular matrices. Laminin is vital for the maintenance and survival of tissues. Defective laminins can cause muscles to form improperly, leading to a form of muscular dystrophy, lethal skin blistering disease (junctional epidermolysis bullosa) and defects of the kidney filter (nephrotic syndrome).

A “stratified tissue” consists of several layers of cells. The vertebrate retina has ten distinct layers:
Inner limiting membrane—basement membrane elaborated by Muller cells.
Nerve fibre layer—axons of the ganglion cell bodies (note that a thin layer of Muller cell footplates exists between this layer and the inner limiting membrane).
Ganglion cell layer—contains nuclei of ganglion cells, the axons of which become the optic nerve fibres, and some displaced amacrine cells.
Inner plexiform layer—contains the synapse between the bipolar cell axons and the dendrites of the ganglion and amacrine cells.
Inner nuclear layer—contains the nuclei and surrounding cell bodies (perikarya) of the amacrine cells, bipolar cells, and horizontal cells.
Outer plexiform layer—projections of rods and cones ending in the rod spherule and cone pedicle, respectively. These make synapses with dendrites of bipolar cells and horizontal cells. In the macular region, this is known as the Fiber layer of Henle.
Outer nuclear layer—cell bodies of rods and cones.
External limiting membrane—layer that separates the inner segment portions of the photoreceptors from their cell nuclei.
Inner segment/outer segment layer—inner segments and outer segments of rods and cones. The outer segments contain a highly specialized light-sensing apparatus.
Retinal pigment epithelium—single layer of cuboidal epithelial cells (with extrusions not shown in diagram). This layer is closest to the choroid, and provides nourishment and supportive functions to the neural retina.

The “lens” is a transparent, biconvex structure in the eye that, along with the cornea, helps to refract light to be focused on the retina. The lens, by changing shape, functions to change the focal distance of the eye so that it can focus on objects at various distances, thus allowing a sharp real image of the object of interest to be formed on the retina. This adjustment of the lens is known as accommodation. Given its role in light refraction and its medical vulnerability, there is a need to provide improved approaches in order to regenerate, replenish or replace (e.g. by transplantation) lens cells or tissues. The lens is part of the anterior segment of the human eye. In front of the lens is the iris, which regulates the amount of light entering into the eye. The lens is suspended in place by the suspensory ligament of the lens, a ring of fibrous tissue that attaches to the lens at its equator and connects it to the ciliary body. Posterior to the lens is the vitreous body, which, along with the aqueous humour on the anterior surface, bathes the lens. The lens has an ellipsoid, biconvex shape. The anterior surface is less curved than the posterior. In the adult, the lens is typically circa 10 mm in diameter and has an axial length of about 4 mm, though it is important to note that the size and shape can change due to accommodation and because the lens continues to grow throughout a person's lifetime. The lens has three main parts: the lens capsule, the lens epithelium, and the lens fibers. The lens capsule forms the outermost layer of the lens and the lens fibers form the bulk of the interior of the lens. The cells of the lens epithelium, located between the lens capsule and the outermost layer of lens fibers, are found only on the anterior side of the lens. The lens itself lacks nerves, blood vessels, or connective tissue.

The “lens capsule” is a smooth, transparent basement membrane that completely surrounds the lens. The capsule is elastic and is composed of collagen. It is synthesized by the lens epithelium and its main components are Type IV collagen and sulfated glycosaminoglycans (GAGs). The capsule is very elastic and so allows the lens to assume a more globular shape when not under the tension of the zonular fibers (also called suspensory ligaments), which connect the lens capsule to the ciliary body. The capsule varies from 2 to 28 micrometres in thickness, being thickest near the equator and thinnest near the posterior pole.

The “lens epithelium”, located in the anterior portion of the lens between the lens capsule and the lens fibers, is a simple cuboidal epithelium. The cells of the lens epithelium regulate most of the homeostatic functions of the lens. As ions, nutrients, and liquid enter the lens from the aqueous humor, Na+/K+-ATPase pumps in the lens epithelial cells pump ions out of the lens to maintain appropriate lens osmotic concentration and volume, with equatorially positioned lens epithelium cells contributing most to this current. The activity of the Na⁺/K⁺-ATPases keeps water and current flowing through the lens from the poles and exiting through the equatorial regions. The cells of the lens epithelium also serve as the progenitors for new lens fibers. It constantly lays down fibers in the embryo, fetus, infant, and adult, and continues to lay down fibers for lifelong growth.

The “lens fibers” form the bulk of the lens. They are long, thin, transparent cells, firmly packed, with diameters typically 4-7 micrometres and lengths of up to 12 mm long. The lens fibers stretch lengthwise from the posterior to the anterior poles and, when cut horizontally, are arranged in concentric layers rather like the layers of an onion. If cut along the equator, it appears as a honeycomb. The middle of each fiber lies on the equator. These tightly packed layers of lens fibers are referred to as laminae. The lens fibers are linked together via gap junctions and interdigitations of the cells that resemble “ball and socket” forms. The lens is split into regions depending on the age of the lens fibers of a particular layer. Moving outwards from the central, oldest layer, the lens is split into an embryonic nucleus, the fetal nucleus, the adult nucleus, and the outer cortex. New lens fibers, generated from the lens epithelium, are added to the outer cortex. Mature lens fibers have no organelles or nuclei.

The “cornea” is the transparent front part of the eye that covers the iris, pupil, and anterior chamber. The cornea, with the anterior chamber and lens, refracts light, with the cornea accounting for approximately two-thirds of the eye's total optical power. In humans, the refractive power of the cornea is approximately 43 dioptres. The cornea can be reshaped by surgical procedures such as LASIK. However, there is a need to provide improved approaches in order to regenerate, replenish or replace (e.g. by transplantation) cornea cells or tissues. While the cornea contributes most of the eye's focusing power, its focus is fixed. Accommodation (the refocusing of light to better view near objects) is accomplished by changing the geometry of the lens. The cornea has unmyelinated nerve endings sensitive to touch, temperature and chemicals; a touch of the cornea causes an involuntary reflex to close the eyelid. Because transparency is of prime importance, the healthy cornea does not have or need blood vessels within it. Instead, oxygen dissolves in tears and then diffuses throughout the cornea to keep it healthy. Similarly, nutrients are transported via diffusion from the tear fluid through the outside surface and the aqueous humour through the inside surface. Nutrients also come via neurotrophins supplied by the nerves of the cornea. In humans, the cornea has a diameter of about 11.5 mm and a thickness of 0.5-0.6 mm in the centre and 0.6-0.8 mm at the periphery. Transparency, avascularity, the presence of immature resident immune cells, and immunologic privilege makes the cornea a very special tissue. The most abundant soluble protein in mammalian cornea is albumin. The human cornea borders with the sclera via the corneal limbus. In lampreys, the cornea is solely an extension of the sclera, and is separate from the skin above it, but in more advanced vertebrates it is always fused with the skin to form a single structure, albeit one composed of multiple layers. In fish, and aquatic vertebrates in general, the cornea plays no role in focusing light, since it has virtually the same refractive index as water. The human cornea has five layers (possibly six, if the Dua's layer is included). Corneas of other primates have five known layers. The corneas of cats, dogs, wolves, and other carnivores only have four. From the anterior to posterior the layers of the human cornea are:

“Corneal epithelium”: an exceedingly thin multicellular epithelial tissue layer (non-keratinized stratified squamous epithelium) of fast-growing and easily regenerated cells, kept moist with tears. Irregularity or edema of the corneal epithelium disrupts the smoothness of the air/tear-film interface, the most significant component of the total refractive power of the eye, thereby reducing visual acuity. It is continuous with the conjunctival epithelium, and is composed of about 6 layers of cells which are shed constantly on the exposed layer and are regenerated by multiplication in the basal layer.

“Bowman's layer” (also known as the anterior limiting membrane): when discussed in lieu of a subepithelial basement membrane, Bowman's Layer is a tough layer composed of collagen (mainly type I collagen fibrils), laminin, nidogen, perlecan and other HSPGs that protects the corneal stroma. When discussed as a separate entity from the subepithelial basement membrane, Bowman's Layer can be described as an acellular, condensed region of the apical stroma, composed primarily of randomly organized yet tightly woven collagen fibrils. These fibrils interact with and attach onto each other. This layer is eight to 14 micrometres (μm) thick and is absent or very thin in non-primates.

“Corneal stroma” (also substantia propria): a thick, transparent middle layer, consisting of regularly arranged collagen fibers along with sparsely distributed interconnected keratocytes, which are the cells for general repair and maintenance. They are parallel and are superimposed like book pages. The corneal stroma consists of approximately 200 layers of mainly type I collagen fibrils. Each layer is 1.5-2.5 μm. Up to 90% of the corneal thickness is composed of stroma. There are 2 theories of how transparency in the cornea comes about: The lattice arrangements of the collagen fibrils in the stroma. The light scatter by individual fibrils is cancelled by destructive interference from the scattered light from other individual fibrils. The spacing of the neighboring collagen fibrils in the stroma must be <200 nm for there to be transparency.

“Descemet's membrane” (also posterior limiting membrane): a thin acellular layer that serves as the modified basement membrane of the corneal endothelium, from which the cells are derived. This layer is composed mainly of collagen type IV fibrils, less rigid than collagen type I fibrils, and is around 5-20 μm thick, depending on the subject's age. Just anterior to Descemet's membrane, a very thin and strong layer, Dua's layer, 15 microns thick and able to withstand 1.5 to 2 bars of pressure.

“Corneal endothelium”: a simple squamous or low cuboidal monolayer, approx. 5 μm thick, of mitochondria-rich cells. These cells are responsible for regulating fluid and solute transport between the aqueous and corneal stromal compartments. (The term endothelium is a misnomer here. The corneal endothelium is bathed by aqueous humor, not by blood or lymph, and has a very different origin, function, and appearance from vascular endothelia.) Unlike the corneal epithelium, the cells of the endothelium do not regenerate. Instead, they stretch to compensate for dead cells which reduces the overall cell density of the endothelium, which affects fluid regulation. If the endothelium can no longer maintain a proper fluid balance, stromal swelling due to excess fluids and subsequent loss of transparency will occur and this may cause corneal edema and interference with the transparency of the cornea and thus impairing the image formed. Iris pigment cells deposited on the corneal endothelium can sometimes be washed into a distinct vertical pattern by the aqueous currents—this is known as Krukenberg's Spindle.

The cornea is one of the most sensitive tissues of the body, as it is densely innervated with sensory nerve fibres via the ophthalmic division of the trigeminal nerve by way of 70-80 long ciliary nerves. Research suggests the density of pain receptors in the cornea is 300-600 times greater than skin and 20-40 times greater than dental pulp, making any injury to the structure excruciatingly painful. The ciliary nerves run under the endothelium and exit the eye through holes in the sclera apart from the optic nerve (which transmits only optic signals). The nerves enter the cornea via three levels; scleral, episcleral and conjunctival. Most of the bundles give rise by subdivision to a network in the stroma, from which fibres supply the different regions. The three networks are, midstromal, subepithelial/sub-basal, and epithelial. The receptive fields of each nerve ending are very large, and may overlap. Corneal nerves of the subepithelial layer terminate near the superficial epithelial layer of the cornea in a logarithmic spiral pattern. The density of epithelial nerves decreases with age, especially after the seventh decade.

“Alpha-crystallin B” chain is a protein that in humans is encoded by the CRYAB gene. It is part of the small heat shock protein family and functions as molecular chaperone that primarily binds misfolded proteins to prevent protein aggregation, as well as inhibit apoptosis and contribute to intracellular architecture. Post-translational modifications decrease the ability to chaperone. Mutations in CRYAB cause different cardiomyopathies and skeletal myopathies. In addition, defects in this gene/protein have been associated with cancer and neurodegenerative diseases such as Alzheimer's disease and Parkinson's disease. CRYAB is provided by the present invention, e.g. for being expressed in the (optic vesicle) 3D brain organoid according to the invention.

Crystallins are separated into two classes: taxon-specific, or enzyme, and ubiquitous. The latter class constitutes the major proteins of vertebrate eye lens and maintains the transparency and refractive index of the lens. Since lens central fiber cells lose their nuclei during development, these crystallins are made and then retained throughout life, making them extremely stable proteins. Mammalian lens crystallins are divided into alpha, beta, and gamma families; beta and gamma crystallins are also considered as a superfamily. Alpha and beta families are further divided into acidic and basic groups.

Seven protein regions exist in crystallins: four homologous motifs, a connecting peptide, and N- and C-terminal extensions. Alpha crystallins are composed of two gene products: alpha-A and alpha-B, for acidic and basic, respectively. These heterogeneous aggregates consist of 30-40 subunits; the alpha-A and alpha-B subunits have a 3:1 ratio, respectively.

“Beta-crystallin B3” is a protein that in humans is encoded by the CRYBB3 gene. CRYBB3 is provided by the present invention, e.g. for being expressed in the (optic vesicle) 3D brain organoid according to the invention.

Seven protein regions exist in crystallins: four homologous motifs, a connecting peptide, and N- and C-terminal extensions. Beta-crystallins, the most heterogeneous, differ by the presence of the C-terminal extension (present in the basic group, none in the acidic group). Beta-crystallins form aggregates of different sizes and are able to self-associate to form dimers or to form heterodimers with other beta-crystallins. This gene, a beta basic group member, is part of a gene cluster with beta-A4, beta-B1, and beta-B2.

“Opticin” is a protein that in humans is encoded by the OPTC gene. OPTC is provided by the present invention, e.g. for being expressed in the (optic vesicle) 3D brain organoid according to the invention. Opticin belongs to class III of the small leucine-rich repeat protein (SLRP) family. Members of this family are typically associated with the extracellular matrix. Opticin is present in significant quantities in the vitreous of the eye and also localizes to the cornea, iris, ciliary body, optic nerve, choroid, retina, and fetal liver. Opticin may noncovalently bind collagen fibrils and regulate fibril morphology, spacing, and organization. The opticin gene is mapped to a region of chromosome 1 that is associated with the inherited eye diseases age-related macular degeneration (AMD) and posterior column ataxia with retinosa pigmentosa (AXPC1).

“Keratin 3 (KRT3)” also known as cytokeratin 3 is a protein that in humans is encoded by the KRT3 gene. Keratin 3 is a type II cytokeratin. It is specifically found in the corneal epithelium together with keratin 12. KRT3 is provided by the present invention, e.g. for being expressed in the (optic vesicle) 3D brain organoid according to the invention. Mutations in the KRT3 encoding this protein have been associated with Meesmanns Corneal Dystrophy. Thus, the present invention is suitable to provide a treatment for Meesmanns Corneal Dystrophy.

“Purkinje cell protein 4” is a protein that in humans is encoded by the PCP4 gene. Also known as PEP-19, PCP4 is a 7.6 kDa protein with an IQ-motif that binds to calmodulin (CaM). PCP4 is abundant in Purkinje cells of the cerebellum, and plays an important role in synaptic plasticity. PCP4 is provided by the present invention, e.g. for being expressed in the (optic vesicle) 3D brain organoid according to the invention. PCP4 knockout mice have been reported to exhibit impaired locomotor learning and markedly altered synaptic plasticity in cerebellar Purkinje neurons. PCP4 accelerates both the association and dissociation of calcium (Ca²⁺) with calmodulin (CaM), which is postulated to influence the activity of CaM-dependent enzymes, especially CaM kinase II (CaMK-II).

A “neural network” or neural circuit is a population of neurons interconnected by synapses to carry out a specific function when activated. Neural circuits interconnect to one another to form large scale brain networks. A network may be operational between photoreceptors of the retina and brain regions. It may involve a network of photoreceptors, ganglion cells, and bipolar cells and inner organoid regions. In a preferred embodiment, it is characterized by the presence of light sensitive cell types that can respond to light stimulation.

According to the present invention, cells can be counted using any technique known to the skilled person, including those that are recited hereinafter.

A “counting chamber”, also known as hemocytometer, is a microscope slide that is especially designed to enable cell counting. The hemocytometer has two gridded chambers in its middle, which are covered with a special glass slide when counting. A drop of cell culture is placed in the space between the chamber and the glass cover, filling it by capillarity. Looking at the sample under the microscope, the grid is used to manually count the number of cells in a certain area of known size. The separating distance between the chamber and the cover is predefined, thus the volume of the counted culture can be calculated and with it the concentration of cells.

A “Coulter counter” is an appliance that can count cells as well as measure their volume. It is based on the fact that cells show great electrical resistance; in other words, they conduct almost no electricity. In a Coulter counter the cells, swimming in a solution that conducts electricity, are sucked one by one into a tiny gap. Flanking the gap are two electrodes that conduct electricity. When no cell is in the gap, electricity flows unabated, but when a cell is sucked into the gap the current is resisted. The Coulter counter counts the number of such events and also measures the current (and hence the resistance), which directly correlates to the volume of the cell trapped. A similar system is the CASY cell counting technology.

In a “flow cytometer” the cells flow in a narrow stream in front of a laser beam. The beam hits them one by one, and a light detector picks up the light that is reflected from the cells. Flow cytometers have many other abilities, such as analysing the shape of cells and their internal and external structures, as well as measuring the amount of specific proteins and other biochemical in the cells.

At present, stereological cell counting with manual decision for object inclusion according to unbiased stereological counting rules remains the only adequate method for unbiased cell quantification in histologic tissue sections, thus it's not adequate enough to be fully automated.

Culture conditions for neurospheres are characterized by the presence of insulin, a TGFβ/activin/nodal pathway inhibitor and at least one antibiotic. In an alternative embodiment, neurospheres are cultured as before with the exception that no antibiotic is present in order to comply with GMP for later clinical trials.

In a preferred embodiment, neurospheres are cultured in a composition further comprising Pen/Strep and β-Mercaptoethanol. In a more preferred embodiment, neurospheres are cultured in a composition comprising:

TABLE 1

| “Neurosphere | | |
|---|---|---|
| medium”: | | |
| Component: | Final conc. | |
| DMEM/F12 | 48.4% (v/v) | |
| Neural basal medium | 48.4% (v/v) | |
| N2 | 0.4× | |
| B27 w/o Vitamin A | 0.2× | |
| Glutamax | 1× | |
| MEM | 0.5× | |
| Insulin | 0.2755 | μM |
| SB431542 | 2.5 | μM |
| Pen/Strep | 100 | U/ml |
| β-Mercaptoethanol | 5 | μM |

In a preferred embodiment, neurospheres are exposed to a composition comprising i) at least one retinoic acid receptor (RAR) activator, at least one bone morphogenetic protein (BMP) pathway inhibitor and at least one transforming growth factor (TGF) β/activin/nodal pathway inhibitor and Pen/Strep and/or β-Mercaptoethanol. In a more preferred embodiment, neurospheres are exposed to a composition comprising:

TABLE 2

| “Optic vesicle 3D | | |
|---|---|---|
| brain organoid | | |
| medium”: | | |
| Component: | Final conc. | |
| DMEM/F12 | 48.4% (v/v) | |
| Neural basal medium | 48.4% (v/v) | |
| N2 | 0.4× | |
| B27 + RA | 0.2× incl. 60 nMol | |
| | retinol acetate | |
| Glutamax | 1× | |
| MEM | 0.5× | |
| Dorsomorphin | 0.5 | μM |
| Insulin | 0.2755 | μM |
| SB431542 | 5 | μM |
| Pen/Strep | 100 | U/ml |
| β-Mercaptoethanol | 5 | μM |

Using the above-identified method of any of embodiments 1B to embodiment 2B, a invention containing retinal pigment epithelium (RPE) can be generated. For this purpose, the exposure of embodiment 1B is followed by a step wherein the invention is cultured in a medium comprising FBS and/or Pen/Strep and/or β-Mercaptoethanol. In a preferred embodiment, the following medium is used:

TABLE 3

| “RPE medium”: | |
|---|---|
| Component: | Final conc. |
| DMEM/F12 | 87% (v/v) |
| FBS | 10% (v/v) |
| Glutamax | 1× |
| MEM | 1× |
| Pen/Strep | 100 U/ml and 100 μg/ml |
| β-Mercaptoethanol | 5 μM |

DMEM/F12 is a commercially available proprietary basal medium containing essential amino acids, glucose and vitamins. Whenever mentioned in this disclosure, it can be replaced by a basal medium containing essential amino acids, glucose and vitamins.

For later use in clinical trials the GMP compliant “Stem Brew”-medium from Miltenyi could be used.

Neurobasal Medium is a commercially available proprietary basal medium designed for long-term maintenance and maturation of pure pre-natal and embryonic neuronal cells. Whenever mentioned in this disclosure, it can be replaced by a basal medium designed for long-term maintenance and maturation of pure pre-natal and embryonic neuronal cells.

FBS stands for Fetal bovine serum. Whenever mentioned in this disclosure, it can be replaced by fetal serum of another species such as sheep, donkey, horse, pig, cat or dog.

Glutamax is an alternative substrate for L-Glutamate. Whenever mentioned in this disclosure, it can be replaced by L-Glutamate.

A “basement membrane” is a thin matrix that separates the lining of an internal or external surface from underlying tissue or other elements (such as elements related to packaging of a medicinal product). The basement membrane may be a thin, fibrous, extracellular matrix. In another embodiment, it is synthetic. The basement membrane may be composed of two layers, The two layers may attach to each other with collagen VII anchoring fibrils and fibrillin microfibrils. The two layers together are collectively referred to as the basement membrane. In another embodiment, there is only one layer and this layer contains collagen (e.g. collagen VII) and/or fibrillin microfibrils. Integrins may be part of the basement membrane.

“Biodegradable” means that the basement membrane can be decomposed by the action of living organisms, preferably microbes, into water, carbon dioxide, and biomass. The following are non-limiting examples of biodegradable materials:

“Polyhydroxyalkanoates (PHAs)” are a class of biodegradable plastic naturally produced by various micro-organisms (example: Vesiclerividus necator). Specific types of PHAs include poly-3-hydroxybutyrate (PHB), polyhydroxyvalerate (PHV) and polyhydroxyhexanoate (PHH). The biosynthesis of PHA is usually driven by depriving organisms of certain nutrients (e.g. lack of macro elements such as phosphorus, nitrogen, or oxygen) and supplying an excess of carbon sources. PHA granules are then recovered by rupturing the micro-organisms. PHA can be further classified into two types:
scl-PHA from hydroxy fatty acids with short chain lengths including three to five carbon atoms are synthesized by numerous bacteria, including Vesicleriavidus necator and Alcaligenes latus (PHB).
mcl-PHA from hydroxy fatty acids with medium chain lengths including six to 14 carbon atoms, can be made for example, by Pseudomonas putida.

“Polylactic acid (PLA)” is thermoplastic aliphatic polyester synthesized from renewable biomass, typically from fermented plant starch such as from corn, cassava, sugarcane or sugar beet pulp.

“Starch blends” are thermoplastic polymers produced by blending starch with plasticizers. Because starch polymers on their own are brittle at room temperature, plasticizers are added in a process called starch gelatinization to augment its crystallization. While all starches are biodegradable, not all plasticizers are. Thus, the biodegradability of the plasticizer determines the biodegradability of the starch blend. Biodegradable starch blends include starch/polylactic acid, starch/polycaprolactone, and starch/polybutylene-adipate-co-terephthalate.

“Cellulose bioplastics” are mainly the cellulose esters, (including cellulose acetate and nitrocellulose) and their derivatives, including celluloid. Cellulose can become thermoplastic when extensively modified. An example of this is cellulose acetate, which is expensive and therefore rarely used for packaging.

“Lignin-based polymer composites” are bio-renewable natural aromatic polymers with biodegradable properties. Lignin is found as a byproduct of polysaccharide extraction from plant material through the production of paper, ethanol, and more. Lignin is useful due to its low weight material and the fact that it is more environmentally friendly than other alternatives. Lignin is neutral to CO₂ release during the biodegradation process. Other biodegradable plastic processes such as polyethylene terephthalate (PET) have been found to release CO₂ and water as waste products produced by the degrading microorganisms. Lignin contains comparable chemical properties in comparison to current plastic chemicals, which includes reactive functional groups, the ability to form into films, high carbon percentage, and it shows versatility in relation to various chemical mixtures used with plastics. Lignin is also stable, and contains aromatic rings. It is both elastic and viscous yet flows smoothly in the liquid phase. Most importantly lignin can improve on the current standards of plastics because it is antimicrobial in nature.

“Petroleum-based plastics” are derived from petrochemicals, which are obtained from fossil crude oil, coal or natural gas. The most widely used petroleum-based plastics such as polyethylene terephthalate (PET), polyethylene (PE), polypropylene (PP), and polystyrene (PS) are not biodegradable.

“Polyglycolic acid (PGA)” is a thermoplastic polymer and an aliphatic polyester. PGA is often used in medical applications such as PGA sutures for its biodegradability. The ester linkage in the backbone of polyglycolic acid gives it hydrolytic instability. Thus, polyglycolic acid can degrade into its nontoxic monomer, glycolic acid, through hydrolysis. This process can be expedited with esterases. In the body, glycolic acid can enter the tricarboxylic acid cycle, after which can be excreted as water and carbon dioxide.

“Polybutylene succinate (PBS)” is a thermoplastic polymer resin that has properties comparable to propylene. It is used in packaging films for food and cosmetics. In the agricultural field, PBS is used as a biodegradable mulching film. PBS can be degraded by Amycolatopsis sp. HT-6 and Penicillium sp. strain 14-3. In addition, Microbispora rosea, Excellospora japonica and E. viridilutea have been shown to consume samples of emulsified PBS.

“Polycaprolactone (PCL)” has gained prominence as an implantable biomaterial because the hydrolysis of its ester linkages offers its biodegradable properties. It has been shown that firmicutes and proteobacteria can degrade PCL. Penicillium sp. strain 26-1 can degrade high density PCL; though not as quickly as thermotolerant Aspergillus sp. strain ST-01. Species of clostridium can degrade PCL under anaerobic conditions.

“Poly(vinyl alcohol) (PVA, PVOH)” is one of the few biodegradable vinyl polymers that is soluble in water. Due to its solubility in water (an inexpensive and harmless solvent), PVA has a wide range of applications including food packaging, textiles coating, paper coating, and healthcare products.

“Polybutylene adipate terephthalate (PBAT)” is a biodegradable random copolymer.

Overview of the Invention

The present invention is directed to a three-dimensional (3D) brain organoid that is characterized in that it contains both first cells that are neuronal and second cells that are non-neuronal. In a preferred embodiment, the first cells are neuroectodermal cells and the second cells are surface ectodermal cells. In a further preferred embodiment, the invention is an optic vesicle 3D brain organoid. The (optic vesicle) 3D brain organoid may be devoid of vasculature as well as cells related to blood circulation.

The first cells may comprise a structure that represents an optic vesicle. The first cells may comprise retinal pigment epithelium (RPE) cells, photoreceptor cells, amacrine cells, bipolar cells, horizontal cells, ganglion cells, Muller cells or combinations thereof. The first cells may express:
a retinal cell marker selected from the group consisting of Rhodopsin (RHO), Opsin (OPN), Cone-rod homeobox protein (CRX), Recoverin (RCVRN), Neural retina leucine zipper (NRL), Basic Helix-Loop-Helix Family Member E22 (BHLHE22), PR/SET domain 8 (PRDM8), Protein kinase C alpha (PKCα), Atonal BHLH Transcription Factor 7 (ATOH7), Distal-Less Homeobox 2 (DLX2), Syntaxin 1A,
(STX1A), Calbindin 2 (CALB2),
Retinoid Isomerohydrolase (RPE65), cellular retinaldehyde binding protein (CRALBP), ADP Ribosylation Factor Like GTPase 13B (Ar113B), Actin, Bestrophin 1 (BEST1),
Synapsin 1 (SYN1), Myelin binding protein (MBP), class III beta tubulin (TUJ1), Purkinje Cell Protein 4 (PCP4), and/or
Laminin.

In the present invention, the first cells may form a stratified tissue such as a retina-like tissue or a retina.

The second cells may form at least one lens, at least one cornea, or combinations thereof. The at least one lens may express genes selected from the group of lens markers consisting of CRYAB, CRYBB3, and/or the cornea expresses genes selected from the group of cornea markers consisting of OPTC; KRT3. The optic vesicle brain organoids may contain cells with up regulated gene expression for sensory perception. In a preferred embodiment, the up-regulated genes are selected from a group comprising or consisting of:
ABCA4, ATP-binding cassette, sub-family A (ABC1), member 4,
CABP1, Calcium binding protein1 (CABP 1-4) for visual perception,
Unc119, unc-119 lipid binding chaperone,
Tulp1, tubby like protein 1,
Rp1, retinitis pigmentosa 1 (human),
Rgr, retinal G protein coupled receptor,
Rd3, retinal degeneration 3,
Rcvrn, recoverin,
Impg2, interphotoreceptor matrix proteoglycan 2,
Impg1, interphotoreceptor matrix proteoglycan 1,
Guca1b, guanylate cyclase activator 1B, and
Cplx3, complexin 3.
   These genes are important for visual perception.

In another preferred embodiment, the up-regulated genes are selected from a group comprising or consisting of:
CACNB4, calcium channel, voltage-dependent, beta 4 subunit,
CCDC66, coiled-coil domain containing 66,
CDH23, cadherin 23 (otocadherin),
Ush2a, usherin, and
Rpe65, retinal pigment epithelium 65.
   These genes are important for detection of light stimuli involved in visual perception.

In a more preferred embodiment, both embodiments (visual perception and detection of light stimuli) are combined. As a result, for example,
ABCA4, ATP-binding cassette, sub-family A (ABC1), member 4
Unc119, unc-119 lipid binding chaperone
Rgr, retinal G protein coupled receptor
Rd3, retinal degeneration 3
Impg2, interphotoreceptor matrix proteoglycan 2
Guca1b, guanylate cyclase activator 1B
CpIx3, complexin 3
CACNB4, calcium channel, voltage-dependent, beta 4 subunit
CDH23, cadherin 23 (otocadherin), and/or
Rpe65, retinal pigment epithelium 65,
   are within the group of genes that are or may be up-regulated.

However, any other combination of those genes that are disclosed hereinabove is according to the present invention.

The first cells may comprise cells expressing Purkinje cell protein 4 (PCP4) and forming a neural network. In one embodiment, the neural network is connecting various cell types from retina to brain regions. The neural network may show an electrical excitation or inhibition pattern in response to light. A network between photoreceptor of retina to brain region involves a network of photoreceptor cells, ganglion cells, and bipolar cells and inner organoid regions. In a preferred embodiment, it is characterized by the presence of light sensitive cell types that can respond to light stimulation. The light may be normal white light and may be composed of wavelengths of different colors ranging from 400 to 700 nm.

In an embodiment, the present invention also relates to a method comprising the steps of
i) culturing at least one stem cell,
ii) subjecting said at least one stem cell to neural induction leading to neurospheres,
iii) collecting and culturing the neurospheres, and
iv) exposing said neurospheres to a composition comprising at least one retinoic acid receptor (RAR) activator, at least one bone morphogenetic protein (BMP) pathway inhibitor and at least one transforming growth factor (TGF) β/activin/nodal pathway inhibitor,
   wherein the at least one stem cell comprises at most 100,000 cells, preferably between 10 and 90,000 cells, more preferably between 100 and 70,000 cells, even more preferably between 1,000 and 50,000 cells, more preferably between 5,000 and 25,000 cells, and wherein said exposure is made within two weeks, preferably within 2 days to 15 days, more preferably within 3 days to 14 days, even more preferred within 5 to 12 days after neural induction.

As mentioned previously, the first cells may be neuroectodermal cells and the second cells may be surface ectodermal cells. In a further preferred embodiment, the method yields an optic vesicle 3D brain organoid. The exposure is preferably made within 6 to 11 days, 7 to 10 days, 8 to 9 days after neural induction. 8 days is most preferred.

The advantage of this method is that it allows for fast generation of brain organoids. Even more advantageously, the method of the present invention allows for a rapid generation of optic vesicle 3D brain organoids.

This has been completely unexpected in view of the state of the art. Recent iPSC-derived brain organoids recapitulate many aspects of human brain development. Studies in the 18th century by Pander revealed that during embryogenesis, the retinal anlage develops laterally from the diencephalon of the forebrain, protruding as an optic vesicle. The present inventors surprisingly found that brain organoids could also assemble bilaterally symmetric optic vesicles in a topographically restricted manner mimicking embryogenesis and resulting in a biologically correct organization. This aspect of brain organoids to assemble functionally integrated optic vesicles allowing interorgan interactions occurring within a single organoid has not yet been demonstrated.

In the present invention, brain organoids are engineered to develop two bilaterally symmetric ‘eye-like’ structures (optic vesicle brain organoids). These structures are associated with forebrain-like region exhibiting vast cellular diversity and in vivo-like functionality. These organoids display various components of an optic vesicle including cornea, lens, photoreceptors, retinal pigment epithelia, visual projections, and electrically active neuronal networks.

Overexpression of signalling molecules is required to define spatial topography of forebrain in organoids. The present invention goes beyond this because organoids according to the invention do develop bilaterally symmetric optic vesicles from the forebrain-like regions without an artificial induction of signalling centres.

Importantly, these organoids can be generated within 20 to 100 days, 30 to 90 days, 40 to 80 days, 45 to 75 days, 46 to 74 days, 47 to 73 days, 48 to 72 days, 49 to 71 days, or 50 to 70 days. More preferably, these organoids can be generated within 40 to 60 days, 41 to 59 days, 42 to 58 days, 43 to 57 days, 44 to 56 days, 45 to 55 days, 46 to 54 days, 47 to 53 days, 48 to 52 days, or 49 to 51 days. Most preferred is 50 days, which is a time frame that parallels with embryonic retina development and feasible to conduct multiple in vitro experiments. Together, the present invention demonstrates that these engineered brain organoids have the surprising ability to assemble brain-associated primitive sensory organs in a topographically restricted manner allowing interorgan interaction studies within a single organoid. Therefore, the present invention provides significant advances to the field of stem cell-based technologies, tissue engineering, and therapy.

The at least one stem cell according to the present invention may be a pluripotent stem cell, an embryonic stem cell, an adult stem cell, a neural stem cell or an embryonic germ cell. Preferably, the at least one stem cell is selected from the group comprising an induced pluripotent stem cell (iPSC), an embryonic stem cell (ESC), a very small embryonic-like stem cell (VSEL), an amniotic fluid stem cell (AFSC), a marrow-isolated adult multilineage inducible cell (MIAMI), a multipotent adult precursor cell (MAPC) or an unrestricted somatic stem cell (USSC). The at least one stem cell may be derived from a human. Preferably, the at least one stem cell is derived from an HLA-homozygous human which is suitable for wide group of human patients. The present invention relates to a 3D brain organoid (optionally an optic vesicle 3D brain organoid) that contains and/or is derived from at least one stem cell as specified hereinbefore.

The neurospheres as recited above may be cultured in a composition comprising insulin, a TGFβ/activin/nodal pathway inhibitor and at least one antibiotic. Alternatively, they may be cultured without any antibiotic within GMP compliance for later clinical trials. Culture conditions are further specified in the definitions, embodiments, and examples. The present invention relates to a 3D brain organoid (optionally an optic vesicle 3D brain organoid) that contains and/or is derived from neurospheres as specified hereinbefore.

The at least one retinoic acid receptor (RAR) activator used in the present invention may be retinol acetate or a derivative thereof, or a semi-synthetic or a synthetic analogue of retinol acetate, or a derivative of such an analogue. Preferably, the at least one retinoic acid receptor (RAR) activator is used in a concentration of greater than 0 and less or equal to 450 nM. In a preferred embodiment, the RAR concentration is between 5 and 430 nM, 10 and 410 nM, 15 and 400 nM, or 20 and 390 nM. More preferably, the RAR concentration is between 2.5 and 200 nM, 5 and 190 nM, 7.5 and 180 nM, 10 and 170 nM, 12.5 and 160 nM, 15 and 150 nM, 17.5 and 140 nM, 20 and 130 nM, 22.5 and 120 nM, 25 and 110 nM, 27.5 and 100 nM, 30 and 90 nM, 32.5 and 80 nM, or 35 and 70 nM. Preferred RAR concentrations are, for example: 5, 10, 15, 20, 25, 30, 35, 40, 45, 50, 55, 60, 65, 70, 75, 80, 85, 90, 95, 100, 105, 110, 115, or 120 nM. A concentration of more than 30 and less than 90 nM is most preferred, in particular more than 40 and less than 80 nM. A concentration between 50 and 70 nM is even more preferred, e.g. 51 to 69 nM, 52 to 68 nM, 53 to 67 nM, 54 to 66 nM, 55 to 65 nM, 56 to 64 nM, 57 to 63 nM, 58 to 62 nM, 59 to 61 nM. A RAR concentration of 60 nM is most preferred. The present invention relates to a 3D brain organoid (optionally an optic vesicle 3D brain organoid) that is obtained and/or cultured in the presence of at least one retinoic acid receptor (RAR) activator as specified hereinbefore.

For example, the following results can be achieved when cells of the cell line IMR90 are exposed to retinoic acid:

TABLE 4

| Retinoic acetate | Percentage of organoids |
|---|---|
| (nM) | with optic vesicles (%) |
| 0 | Less than 1 |
| 40 | 21 |
| 60 | 80 |
| 100 | 15 |
| 150 | 10 |
| 200 | 10 |
| 250 | 8 |

The at least one BMP pathway inhibitor used in the present invention may be dorsomorphin or a derivative thereof, or a semi-synthetic or a synthetic analogue of dorsomorphin, or a derivative of such an analogue. Preferably, said at least one BMP pathway inhibitor is used in a concentration of 0.05 to 5 μM. The present invention relates to a 3D brain organoid (optionally an optic vesicle 3D brain organoid) that is obtained and/or cultured in the presence of at least one BMP pathway inhibitor as specified hereinbefore.

The at least one TGFβ/activin/nodal pathway inhibitor used in the present invention may be SB431542 or a derivative thereof, or a semi-synthetic or a synthetic analogue of SB431542, or a derivative of such an analogue. Preferably, the at least one TGFβ/activin/nodal pathway inhibitor is used in a concentration of 0.5 to 50 μM. The present invention relates to a 3D brain organoid (optionally an optic vesicle 3D brain organoid) that is obtained and/or cultured in the presence of at least one TGFβ/activin/nodal pathway inhibitor as specified hereinbefore.

The neurospheres used in the present invention may be exposed to a composition comprising 0.05 to 5 82 M dorsomorphin, 0.05 to 1.5 μM insulin, 0.5 to 50 μM SB431542, 100 U/ml Pen/Strep, and 0.5 to 5 μM β-Mercaptoethanol. The present invention relates to a 3D brain organoid (optionally an optic vesicle 3D brain organoid) that is obtained and/or cultured in the presence of a composition comprising 0.05 to 5 μM dorsomorphin, 0.05 to 1.5 μM insulin, 0.5 to 50 μM SB431542, 100 U/ml Pen/Strep, and 0.5 to 5 μM β-Mercaptoethanol.

3D brain organoids may be obtained by the method described herein. Furthermore, a tissue may be isolated from a 3D brain organoid obtained by the method of the invention. The tissue may be a monolayer. Preferably, the tissue comprises cells that are characterized by:
predominantly apical PEDF and basal BEST expression, and/or
PMEL17 expression, and/or
ZO1 expression, and/or
Absence of Ki67 expression, and/or
Expression of cellular retinaldehyde binding protein (CRALBP), and/or
Expression of MITF, and/or
Expression of OTX2, and/or
Secretion of PEDF, and/or
Cobblestone morphology, and/or
an ultrastructure comprising tight junctions marked by phalloidin, basal infoldings, apical microvilli, and melanin granules (Pigments).

The tissue may be an artificial retinal pigment epithelium (RPE). Thus, the present invention relates to a procedure wherein a tissue as specified hereinbefore is obtained from a 3D brain organoid (optionally an optic vesicle 3D brain organoid). The tissue obtained in this way may be cryopreserved as an intermediate cell bank.

An artificial RPE of the invention as described above and a synthetic basement membrane or a tissue according to the invention and a synthetic basement membrane may be combined to form a patch. In an alternative embodiment (Eₐₗₜ) the basement membrane is not synthetic but rather made of extracellular matrix. Thus, the present invention relates to a procedure wherein a patch as specified hereinbefore is obtained from a 3D brain organoid (optionally an optic vesicle 3D brain organoid). The patch obtained in this way may be used for medical purposes, for therapy, or surgery.

The synthetic basement membrane may comprise a polyester membrane and/or parylene C. Alternatively or additionally, the synthetic basement membrane may comprise a polyethylene terephthalate (PET) membrane, optionally coated in plasma-derived vitronectin. Preferably, the synthetic basement membrane is biodegradable. Without being intended to be bound by theory, it is believed that a biodegradable scaffold provides suitable material for RPE cells or other tissue according to the invention to secrete extracellular matrix (ECM) to form a polarized monolayer. If the scaffold degrades, ECM and cells would constitute a native-like (RPE) tissue that would enhance the possibility of long-term integration of the (iRPE) patch in the host (e.g. the patients' eyes).

In another or additional aspect of the invention, the synthetic basement membrane may comprise a scaffold manufactured using poly(lactic-co-glycolic acid) (PLGA), preferably (50:50 lactic acid/glycolic acid, four midpoint 1.0 dl/g), with 350 nm mean fiber diameter. The present invention relates to a 3D brain organoid (optionally an optic vesicle 3D brain organoid) or parts thereof (such as tissues or cells) that advantageously combines with this synthetic basement membrane or any other membrane specified hereinbefore.

A pharmaceutically acceptable composition according to the invention comprises the (optic vesicle) 3D brain organoid according to the invention and/or tissues and/or cells and/or molecules derived therefrom. The present invention relates to procedure for producing a pharmaceutically acceptable composition, using the 3D brain organoid (optionally an optic vesicle 3D brain organoid). The pharmaceutically acceptable composition may be used in the treatment of diseases associated with visual impairment including retinal disorders. Preferably, the pharmaceutical composition is GMP/ATMP-acceptable. The diseases may be selected from age-related macular degeneration (AMD), retinitis pigmentosa (RP), Leber congenital amaurosis (LCA), Stargardt disease, choroideremia, Usher syndrome, X-linked retinoschisis (XLRS), achromatopsia (ACHM), and glaucoma.

The tissue according to the invention or the patch according to the invention may be used in the treatment of diseases associated with visual impairment. Preferably, the tissue or patch is produced under GMP/ATMP compliance. The disease may be selected from age-related macular degeneration (AMD), retinitis pigmentosa (RP), Leber congenital amaurosis (LCA), Stargardt disease, choroideremia, Usher syndrome, X-linked retinoschisis (XLRS), achromatopsia (ACHM), and glaucoma. The tissue or patch may be transplanted into the eye of a patient, preferably wherein the transplantation is preceded and/or followed by immunosuppression. The immunosuppression may consist of perioperative prednisolone and long-term intraocular steroid implants. The transplantation may be followed by subretinal administration of a Rho kinase inhibitor and/or a ROCK inhibitor such as Y27632.

The (optic vesicle) 3D brain organoid according to the invention may be used in toxicity in vitro assays including bacterial and viral infection tests. It may also be used in modelling retinopathies and eye development. Further, the (optic vesicle) 3D brain organoid according to the invention may further be used in drug screening in vitro assays. These uses have been completely unexpected.

Medical Indications of the Present Invention

The application discloses a variety of unexpected properties and characteristics of the (optic vesicle) 3D brain organoids according to the invention. It is immediately evident that these properties and characteristics argue in favour of the medical uses specified in this application (embodiments 1 E to 7E).

Age-Related Macular Degeneration (AMD):

AMD is the leading cause of blindness in the elderly population, with individuals over 70 years of age at especially high risk; this disease is predicted to affect almost 200 million people by 2020 (. With the aging of the global population, an increased need for healthcare and the associated economic burden result in rising costs for the elderly population. At the early stage, the key sign of AMD is the appearance of drusen, which are visible with a diameter of>25 μm. Drusen are composed of lipids, proteins, small RNAs and lipofuscin granules. Some of these components can induce inflammasome activation, which may exacerbate the degeneration of retinal cells. The late stage of AMD can be categorized into two forms, neovascular wet AMD and non-neovascular dry AMD, also called geographic atrophy. In some of these patients, choriocapillaris changes can be detected, and choriocapillaris endothelial cell death results in ghost vessels. A possible causative factor of choriocapillaris endothelial cell death is the accumulation of the membrane attack complex (MAC). It was further found that macula with more ghost vessels had significantly more sub-RPE deposits than eyes with fewer ghost vessels. However, whether choriocapillaris dropout is preceded by or arises from RPE degeneration is not well understood. In some wet AMD patients, central vision may be lost in a short period of time if choroidal neovascularization or leaky vessels affect the macula. Patients with dry AMD lose their central vision in a relatively slow, progressive manner because of the gradually decreased RPE density. RPE dysfunction and degeneration is common in both forms of the disease.

The pathogenesis of AMD is complex and is associated with both genetic components and environmental factors. Thus far, a number of genetic factors have been found to be associated with AMD. Most of these genes, including complement factor H (CFH), C2, C3, CFB, tolllike receptor 3 (TLR3), and toll-like receptor 4 (TLR4), are involved in the complement pathway or related to the immune system. Some mutations of these genes, such as the Y402H polymorphism of CFH and 412F variant of TLR3, are well documented to be associated with AM D. CFH plays an essential role in the regulation of complement activation, which can be detected in drusen. Mutations in the CFH gene can lead to the destruction of photoreceptor and RPE cells. TLR3 can recognize double-stranded RNA (dsRNA), which is also a component of drusen. Excess dsRNA can activate the immune response in photoreceptor and RPE cells, resulting in retinal degeneration. Environmental factors, such as diet, smoking and light exposure, are also associated with AMD. Thus, this multifactorial disease exhibits diverse phenotypes in a widespread population. Currently, retinal degeneration is an irreversible process, and no radical cure is available. Some treatments targeting neovascularization can slow down the progression of vision loss and disease symptoms. In wet AMD, these treatments include injection of anti-VEGF and surgical removal of the neovascular membrane. Several VEGF treatments, such as bevacizumab (Avastin, Genentech), ranibizumab (Lucentis, Genentech/Novartis) and aflibercept (VEGF Trap-Eye, Regeneron/Bayer), have been shown to be effective for advanced neovascular AMD. However, geographic atrophy, which accounts for the majority of the AMD population, remains incurable. Transplantation of autologous peripheral RPE underneath the macula has provided proof-of-concept evidence that RPE cell replacement can improve visual function, indicating a therapeutic potential for AMD. A limited autologous RPE cell source prevents the widespread application of this method. Stem cells, with the potential to differentiate into almost all cell types, have been used for RPE cell generation in a dish. The relevant approaches are discussed below. Nowadays, it is well accepted that stem cell-derived RPE cells provide numerous sources of exogenous RPE for cell replacement.

Retinitis Pigmentosa

RP is a major cause of blindness in populations below middle age, affecting over 2 million people worldwide. The age of RP patients varies from infancy to middle age, and their phenotypes are highly variable. These patients may experience night blindness to severe vision loss with the development of the disease; by age 40, most patients are legally blind. Night blindness is caused by primary loss of rod photoreceptor cells, which are responsible for peripheral and low-light vision. With the progression of the disease, subsequent cone degeneration leads to central and color vision loss. RP is an inherited retinal degenerative disease that exhibits exceptional genetic heterogeneity. RP disease-causing genes can be categorized into eight main classes based on their biological functions, including the phototransduction cascade, vitamin A metabolism, RNA intron-splicing factors, structural or cytoskeletal roles, synaptic interaction, intracellular trafficking, pH regulation, cilia maintenance and RPE phagocytosis. At present, more than 4500 mutations in approximately 70 disease-causing genes have been identified. Recently, new RP-associated genes were found following the application of next-generation sequencing. These findings provide a molecular basis for improving approaches for clinical genetic diagnosis. An investigation of a large cohort in a single North American eye clinic found disease-causing genotypes with a sensitivity of 76% using genetic testing. Despite the increasing number of identified disease-causing genes, not all RP patients can be revealed the genetic predispositions in known genes. In most cases, vision loss in RP is due to photoreceptor cell or RPE cell degeneration caused by gene mutations. Since the underlying mechanism of cell death is largely unclear, few effective treatments have been developed. Dietary supplementation is highly recommended by many physicians for patients with early-stage RP to slow disease progression. Several other strategies to treat advanced RP patients are being assessed in clinical trials. Transplantation of an artificial retina, the Argus II Retinal Stimulation System, has been approved by the FDA and has improved long-term visual function in late-stage RP patients. Another method involves the transplantation of ARPE-19 cells expressing neurotrophic factors (such as ciliary neurotrophic factor, CNTF). Gene therapy to target patients with specific mutations using AAV2 has been applied in the treatment of Leber's congenital amaurosis (LCA), which is caused by an RPE65 gene mutation. The CRISPR/Cas9 system also has great potential as a therapeutic treatment for retinal degenerative diseases. An AAV-based CRISPR/Cas9 system was used in a retinal degeneration mouse model and improved photoreceptor survival by disrupting Nrl expression in postmitotic photoreceptors. In a rat model carrying the dominant S334ter mutation in the Rhodopsin gene, subretinal injection of guide RNA/Cas9 plasmid could prevent retinal degeneration and improve visual function. Relative to these methods, cell-replacement therapy is considered a competitive approach for widespread application. For patient-derived iPSCs, CRISPR/Cas9 genome editing strategies can also be integrated to correct genetic defects before transplantation. Taken together, regeneration strategies for RP via replenishment of prosthesis, neurotrophic factors, forced gene expression have been extended to human trials, and gene editing or cell replacement is underway on pre-clinical studies.

Stargardt's Disease

Stargardt's disease, characterized by the deposition of lipofuscin-like substance in RPE with secondary photoreceptor cell death in the macula, is an inherited blinding disease. Mutations most frequently occur in the ABCR gene (also known as ABCA4), which consists of 50 exons and encodes a 2273-amino-acid protein. ABCR is a retina-specific ATP-binding cassette (ABC) protein that plays a key role in retinoid cycling. Dysfunction of this gene leads to the accumulation of N-retinylidene-N-retinyl-ethanolamine (A2E), a major component of lipofuscin, in RPE cells. The accumulation of A2E appears to be responsible for the loss of photoreceptor and RPE cells, leading to severe vision loss in patients. In a mouse model of recessive Stargardt's macular degeneration, treatment with isotretinoin inhibits A2E formation and lipofuscin accumulation. Thus far, however, no effective treatment is clinically available to slow the progression of Stargardt's patients. It has been recently proposed that cell replacement therapy may compensate for the current lack of therapies for Stargardt's disease. In the last decade, two studies have made the attempt to transplant human ESC-derived RPE into subretinal space of patients with Stargardt's macular dystrophy, indicating a promising treatment for this disease.

Glaucoma

Glaucoma is the most prevalent neurodegenerative disease, affecting more than 70 million people worldwide. The etiology of glaucoma is relatively complex, and signs of glutamate toxicity, oxidative stress, and reactive glial changes are observed in glaucoma patients. Intraocular pressure is determined by the dynamic flow and drainage of aqueous humor secreted by the ciliary body. The trabecular meshwork and uveoscleral outflow pathways are the two independent pathways for aqueous humor drainage. Both dysfunction of the trabecular meshwork and anatomic location abnormality of iris blocking the trabecular meshwork can lead to two different kinds of glaucoma, designated open-angle glaucoma and angle-closure glaucoma. Presently, control of intraocular pressure by medication and eye surgery are the main verified treatments slowing down the disease progression. Although these treatments can delay the development of the disease, they cannot prevent neurodegeneration eventually and vision impairment resulting from optic nerve damage and RGC loss is irreversible. Bone marrow MSCs can exert neuroprotective effects and promote the survival of RGCs in aged rats with glaucoma. Although neuroprotection of MSCs can provide another potential treatment option for glaucoma, it is much more difficult to regenerate RGC and optic nerve beyond paracrine effect in cell replacement strategy.

The Claimed Medical Uses in View of Common General Knowledge

Organoids can be used to model diseases, for example modelling neurodevelopmental disorders with cerebral organoids. These types of disease models can then be used for testing drug efficacy in vitro before moving to animal models. Drug compounds can be tested for toxicity and metabolic profile in liver organoids. And finally, organoids could be made from patient cells to provide autologous transplant solutions Age-related macular degeneration (AMD) is a leading cause of blindness among elderly.

Previous success with surgical procedures transplanting autologous retinal pigment epithelium (RPE)/choroid graft obtained from the periphery of the same patients' eyes has provided the critical proof of principle needed to develop pluripotent stem cell—derived RPE-based cell therapies.

Sharma et al. (Clinical-grade stem cell-derived retinal pigment epithelium patch rescues retinal degeneration in rodents and pigs. Sci Transl Med. 2019 Jan. 16; 11(475)) developed oncogenic mutation-free clinical-grade induced pluripotent stem cells (iPSCs) from three AMD patients and differentiated them into clinical-grade iPSC-RPE patches on biodegradable scaffolds. Functional validation of clinical-grade iPSC-RPE patches revealed specific features that distinguished transplantable from nontransplantable patches. Compared to RPE cells in suspension, their biodegradable scaffold approach improved integration and functionality of RPE patches in rats and in a porcine laser-induced RPE injury model that mimics AMD-like eye conditions. Their results suggest that the in vitro and in vivo preclinical functional validation of iPSC-RPE patches developed by them might ultimately be useful for evaluation and optimization of autologous iPSC-based therapies.

Further, a clinical-grade manufacturing process was described that is efficient and reproducible, and generates safe and efficacious clinical product. The manufacturing process was developed using CD34+ cells of three advanced-stage AMD (GA) patients. They generated passage 10 working banks of clinical-grade iPSCs and validated up to three banks per patient for iPSC critical quality attributes. In addition to their pluripotency and correct G-band karyotyping, they focused on two safety attributes: loss of reprogramming plasmid and oncogene sequencing. Thus, Sharma et al successfully demonstrated clinical-grade differentiation of iPSCs into a mature and polarized RPE patch on a biodegradable PLGA-based scaffold.

Additionally, a patch was described comprising a fully differentiated, human embryonic stem cell (hESC)-derived RPE monolayer on a coated, synthetic basement membrane, using a purpose-designed microsurgical tool, into the subretinal space of one eye in each of two patients with severe exudative AMD. Primary endpoints were incidence and severity of adverse events and proportion of subjects with improved best-corrected visual acuity of 15 letters or more. They reported successful delivery and survival of the RPE patch by biomicroscopy and optical coherence tomography, and a visual acuity gain of 29 and 21 letters in the two patients, respectively, over 12 months. Only local immunosuppression was used long-term. They also presented the preclinical surgical, cell safety and tumorigenicity studies leading to trial approval. Their work supports the feasibility and safety of hESC-RPE patch transplantation as a regenerative strategy for AMD.

It was pointed out that all methods of RPE transplantation required creation of a retinal detachment so that cells can be delivered to the space between Bruch's membrane and the neural retina. Retinal detachment affects synapses in the outer plexiform layer. Synaptic injury begins with retraction of rod presynaptic terminals toward their cell bodies. Axonal retraction results in disjunction of the first synapse in the visual pathway as the rod presynaptic terminal disconnects from the postsynaptic bipolar cell dendrite. Retinal detachment also disrupts cone terminals, which lose their synaptic invaginations and normal connections with bipolar cells. Shortly after rod terminal retraction, bipolar cell dendrites extend into the outer nuclear layer, and horizontal cells exhibit sprouting and sometimes extend into the subretinal space. Rod axon retraction and bipolar and horizontal cell sprouting occur in humans after retinal detachment. Retinal detachment is also associated with some degree of neuronal apoptosis even if the detachment is relatively brief. Retinal reattachment allows photoreceptor outer segments to regrow but does not restore retinal synaptic structure completely.

It was further shown that synaptic retraction is associated with a significant increase in RhoA-GTP formation, and this biochemical change begins within minutes after retinal injury or detachment. Synaptic retraction can be blocked using Rho A antagonists. RhoA activates Rhoassociated protein kinase (ROCK). ROCK belongs to the AGC (PKA/PKG/PKC) family of serine-threonine kinases. ROCK is involved mainly in regulating the shape and movement of cells by acting on the cytoskeleton. Through its actions on LIM kinase and cofilin, ROCK increases actin depolymerization. In addition, by phosphorylating myosin light chain, ROCK induces actin binding to myosin II and contractility increases. Human ROCK1 is a major downstream effector of the small GTPase RhoA. Thus, one can block the effects of RhoA with ROCK inhibitors.

Using an in vivo model of retinal detachment in pigs, Wang et al. found that rod-bipolar synaptic disjunction not only occurs in the area of the detachment, it also occurs in attached retina millimeters away from the area of detachment. The implication of this finding is that even if surgeons attempt to spare the fovea from detachment when delivering drugs, cells, or genes to the subretinal space, an extrafoveal detachment in the macular area is likely to induce synaptic changes in the fovea that may compromise the patient's final vision. In addition, Wang et al. found that rod-bipolar synaptic disjunction could be reduced with subretinal administration of a ROCK inhibitor. Townes-Anderson et al. reported that subretinal injection of fasudil, a ROCK inhibitor that is approved for clinical use for a different indication, also reduces rod-bipolar synaptic disjunction. Intravitreal injection is also effective, which may facilitate clinical use, as intravitreal injections are done routinely in an outpatient setting in most retina clinics. Another benefit of ROCK inhibition is that it reduces photoreceptor apoptosis induced by retinal detachment.

Cell therapy can have the objectives of rescue (i.e., modulation of metabolic abnormalities primarily for sight preservation) as well as replacement (i.e., replace cells lost due to injury or disease with the goal of sight restoration as well as preservation). The first clinical trials of RPE transplantation for the late complications of AMD have begun with some preliminary signs of success (e.g., improvement in vision in some patients, anatomic evidence of transplant-host integration with some evidence of host photoreceptor recovery, long-term survival of autologous iPSC transplants without immune suppression) as well as limitations (e.g., limited RPE suspension survival in the AMD eye, limited tolerance for long-term systemic immune suppression in elderly patients, suggestion of uncontrolled cell proliferation in the vitreous cavity). RPE survival on aged and AMD Bruch's membrane can be improved with chemical treatment. This finding establishes the possibility that RPE transplants may survive in AMD eyes without the use of a scaffold, which, if true, may enhance the efficacy of transplants of RPE suspensions in AMD eyes. Delivery of cell suspensions is technically easier and possibly safer than delivery of cells on a scaffold. Nonetheless, these bioactive moieties might also be integrated into scaffolds used to deliver cells to the subretinal space. Retinal detachment, currently used to deliver transplanted RPE cells, induces disjunction of the first synapse in the visual pathway: the photoreceptor-bipolar synapse. This synaptic change occurs even in areas of attached retina near the locus of detachment. Synaptic disjunction and photoreceptor apoptosis associated with retinal detachment can be reduced with Rho kinase inhibitors. Addition of Rho kinase inhibitors may improve retinal function and photoreceptor survival after subretinal delivery of cells either in suspension or on scaffolds.

Generation of Structured Neuro-Retinal Organoids

With its complex morphogenesis, the neural retina, which contains various cell types connected to multiple synaptic junctions, is unlikely to be repaired by transplantation of a single cell type in patients with end-stage retinal degeneration. The replacement of retinal tissue may inspire potential therapies. Attempts have been made to generate neural retina tissue from pluripotent cells in vitro, and an optic vesicle-like structure containing rhodopsin positive cells can be induced in a 2-dimensional (2D) culture system. In the floating retinal organoids, the three neural retina layers exist in layers similar to those in retinas in vivo, and the photoreceptors in the organoid are more mature than those in a 2D culture system.

The 3D cell culture system has been shown to induce the formation of an optic vesicle from mouse and human ESCs (. The ESC-derived optic vesicle contained the major neural retina components, including photoreceptors, RGCs, and bipolar cells. Based on the self-formation in ESC culture, other protocols for optic-vesicle generation have also been developed. With timed BMP4 treatment, the efficiency of Rx-positive epithelium was dramatically increased, and the transition from neural retina tissue to RPE was achieved by inhibiting GSK3 and FGFR. Recently, culture conditions modified by the addition of B27 were used to supplement Neurobasal-A enhanced neurite outgrowth from RGCs in mouse and human ESC models.

Similarly, an optic vesicle with RGC cells can be generated from human iPSCs. The method was modified to generate functional RGCs from human iPSCs, mouse ESCs and mouse iPSCs by combining 3D and 2D cultures; typical RGC action potentials have been recorded in these optic vesicles. A recent study demonstrated that human iPSCs seeded on an engineered scaffold could grow dendritic arbors and functional axons; this approach is promising for clinical translation in the future.

The neural retina develops from the optic vesicle to the optic vesicle, but the mechanism of optic-vesicle organogenesis remains elusive. Notably, reports have shown that 3D culture can induce the formation of an optic vesicle and ciliary epithelium from mouse ESCs and mouse iPSCs, respectively, under the corresponding differentiation conditions. The connecting cilium, located between the inner and outer segment, is a critical structure for material transfer in photoreceptors. The generation of 3D retina with the connecting cilium and outer segments makes this tool a powerful one for modelling RP, and mature photoreceptor cells are expected to benefit for visual rescue after transplantation. For therapeutic application, Reichman et al. (2017) developed a completely defined condition for retinal organoid differentiation from human iPSCs. The removal of animal derivatives is a key improvement for the clinical transplantation of retinal tissue. Even though the optic-vesicle structure can be generated in many laboratories, the presence of photoreceptors with outer-segment discs, electrophysiological properties, and light sensitivity have been reported by only a few groups (Deng et al., 2018; Nakano et al., 2012; Shimada et al., 2017; Zhong et al., 2014) partly due to the prolonged induction in a dish.

During retinal development, the fates of various cell types are controlled by transcription factors in a precise spatiotemporal order. Understanding the profile of transcriptional and epigenetic changes that occur during retinogenesis is expected to assist in optimizing methods for generating more mature retinal organoids in vitro. Kaewkhaw et al. (2015) analyzed the transcriptome of 3D retinal organoids, profiling temporal changes in gene signatures at different stages. Additionally, the identification of specific cell surface markers provides guidelines for the purification of developmental photoreceptors of different stages. These transcriptional data are helpful for delineating gene regulatory networks during retinal development. However, the stage-by-stage regulatory processes during development of real human retina have been unrevealed in the long history. Recently, two studies have filled this gap. Hoshino et al. (2017) provided a comprehensive molecular analysis of fetal retina and characterized the cellular basis of foveal development. Aldiri et al. (2017) focused on the dynamics of epigenetics and transcriptomics during retinogenesis in humans. These studies provide an epigenetic and transcriptional map that serves as a reference for molecular staging of human stem-cell derived retinal organoids.

### Embodiments of the invention

### A. Product Embodiments

### Embodiment 1A

1A. Three-dimensional (3D) brain organoid characterized in that the 3D brain organoid contains both first cells that are neuronal and second cells that are non-neuronal. In a preferred embodiment, the first cells are neuroectodermal cells and the second cells are surface ectodermal cells. In a preferred embodiment, the 3D brain organoid is an optic vesicle 3D brain organoid.

### Embodiment 2A

2A. The 3D brain organoid according to embodiment 1A, being devoid of vasculature as well as cells related to blood circulation.

### Embodiment 3A

3A. The 3D brain organoid according to embodiment 1A or 2A, wherein the first cells comprise a structure that represents an optic vesicle.

### Embodiment 4A

4A. The 3D brain organoid according to any one of embodiments 1A to 4A, wherein the first cells comprise retinal pigment epithelium (RPE) cells, photoreceptor cells, amacrine cells, bipolar cells, horizontal cells, ganglion cells, Muller cells or combinations thereof.

### Embodiment 5A

5A. The 3D brain organoid according to any one of embodiments 1A to 4A, wherein the first cells express:
a retinal cell marker selected from the group consisting of Rhodopsin (RHO), Opsin (OPN), Cone-rod homeobox protein (CRX), Recoverin (RCVRN), Neural retina leucine zipper (NRL), Basic Helix-Loop-Helix Family Member E22 (BHLHE22), PR/SET domain 8 (PRDM8), Protein kinase C alpha (PKCα), Atonal BHLH Transcription Factor 7 (ATOH7), Distal-Less Homeobox 2 (DLX2), Syntaxin 1A (STX1A), Calbindin 2 (CALB2).; and/or
Retinoid Isomerohydrolase (RPE65), cellular retinaldehyde binding protein (CRALBP), ADP Ribosylation Factor Like GTPase 13B (Arl13B), Actin, Bestrophin 1 (BEST1),
Synapsin 1 (SYN1), Myelin binding protein (MBP), class III beta tubulin (TUJ1), Purkinje Cell Protein 4 (PCP4), and/or
Laminin.

### Embodiment 6A

6A. The 3D brain organoid according to any one of embodiments 1A to 5A, wherein the first cells form a stratified tissue.

### Embodiment 7A

7A. The 3D brain organoid according to any one of embodiments 1A to 6A, wherein the second cells form at least one lens, at least one cornea, or combinations thereof.

### Embodiment 8A

8A. The 3D brain organoid according to any one of embodiments 1A to 7A, wherein the at least one lens expresses genes selected from the group of lens markers consisting of CRYAB, CRYBB3, and/or the cornea expresses genes selected from the group of cornea markers consisting of OPTC; KRT3.

### Embodiment 9A

9A. The 3D brain organoid according to any of embodiments 1A to 8A, wherein the optic vesicle 3D brain organoids contain cells with up regulated gene expression for sensory perception.

In a preferred embodiment, the up-regulated genes are selected from a group comprising or consisting of:
ABCA4, ATP-binding cassette, sub-family A (ABC1), member 4
CABP1, Calcium binding protein1 (CABP 1-4) for visual perception
Unc119, unc-119 lipid binding chaperone
Tulp1, tubby like protein 1
Rp1, retinitis pigmentosa 1 (human)
Rgr, retinal G protein coupled receptor
Rd3, retinal degeneration 3
Rcvrn, recoverin
Impg2, interphotoreceptor matrix proteoglycan 2
Impg1, interphotoreceptor matrix proteoglycan 1
Guca1b, guanylate cyclase activator 1B, and/or
Cplx3, complexin 3.
   These genes are important for visual perception.

In another preferred embodiment, the up-regulated genes are selected from a group comprising or consisting of:
CACNB4, calcium channel, voltage-dependent, beta 4 subunit
CCDC66, coiled-coil domain containing 66
CDH23, cadherin 23 (otocadherin)
Ush2a, usherin, and/or
Rpe65, retinal pigment epithelium 65.
   These genes are important for detection of light stimuli involved in visual perception.

In a more preferred embodiment, both embodiments (visual perception and detection of light stimuli) are combined. As a result, for example,
ABCA4, ATP-binding cassette, sub-family A (ABC1), member 4
Unc119, unc-119 lipid binding chaperone
Rgr, retinal G protein coupled receptor
Rd3, retinal degeneration 3
Impg2, interphotoreceptor matrix proteoglycan 2
Guca1b, guanylate cyclase activator 1B
Cplx3, complexin 3
CACNB4, calcium channel, voltage-dependent, beta 4 subunit
CDH23, cadherin 23 (otocadherin), and/or
Rpe65, retinal pigment epithelium 65,
   are within the group of genes that are or may be up-regulated.

However, any other combination of those genes that are disclosed hereinabove is according to the present invention.

### Embodiment 10A

10A. The 3D brain organoid according to any of embodiments 1A to 9A, wherein the first cells comprise cells expressing Purkinje cell protein 4 (PCP4) and forming a neural network. In one embodiment, the neural network is connecting various cell types from retina to brain regions.

### Embodiment 11A

11A. The 3D brain organoid according to embodiment 10A, wherein the neural network shows an electrical excitation or inhibition pattern in response to light. A network as mentioned hereinbefore between photoreceptors of the retina and brain regions involves a network of photoreceptor cells, ganglion cells, and bipolar cells and inner organoid regions. In a preferred embodiment, it is characterized by the presence of light sensitive cell types that can respond to light stimulation. In a preferred embodiment, the light as disclosed in embodiment 11A is normal white light and is composed of wavelengths of different colors ranging from 400 to 700 nm.

### B. Method Embodiments

### Embodiment 1B

1B. A method for producing a 3D brain organoid characterized in that the 3D brain organoid contains both first cells that are neuronal cells and second cells that are surface ectodermal cells, wherein the method comprises the steps of:

i. culturing at least one stem cell,

ii. subjecting said at least one stem cell to neural induction leading to neurospheres,

iii. collecting and culturing the neurospheres, and

iv. exposing said neurospheres to a composition comprising at least one retinoic acid receptor (RAR) activator, at least one bone morphogenetic protein (BMP) pathway inhibitor and at least one transforming growth factor (TGF) β/activin/nodal pathway inhibitor,

wherein the at least one stem cell comprises at most 100,000 cells, preferably between 10 and 90,000 cells, more preferably between 100 and 70,000 cells, even more preferably between 1,000 and 50,000 cells, more preferably between 5,000 and 25,000 cells, and wherein said exposure is made within two weeks, preferably within 2 days to 15 days, more preferably within 3 days to 14 days, even more preferred within 5 to 12 days after neural induction.

In a preferred embodiment, the first cells are neuroectodermal cells and the second cells are surface ectodermal cells.

In a preferred embodiment, the 3D brain organoid is an optic vesicle 3D brain organoid.

In a preferred embodiment, the exposure is made within 6 to 11 days, 7 to 10 days, 8 to 9 days after neural induction. 8 days is most preferred.

The advantage of this method is that it allows for fast generation of brain organoids. Even more advantageously, the method of embodiment 1B allows for a rapid generation of optic vesicle 3D brain organoids.

This has been completely unexpected in view of the state of the art. Recent iPSC-derived brain organoids recapitulate many aspects of human brain development. Studies in the 18th century by Pander revealed that during embryogenesis, the retinal anlage develops laterally from the diencephalon of the forebrain, protruding as an optic vesicle. The present inventors surprisingly found that brain organoids could also assemble bilaterally symmetric optic vesicles in a topographically restricted manner mimicking embryogenesis and resulting in a biologically correct organization. This aspect of brain organoids to assemble functionally integrated optic vesicles allowing interorgan interactions occurring within a single organoid has not yet been demonstrated.

In the present invention, brain organoids are engineered to develop two bilaterally symmetric ‘eye-like’ structures (optic vesicle brain organoids). These structures are associated with forebrain-like region exhibiting vast cellular diversity and in vivo-like functionality. These organoids display various components of an optic vesicle including cornea, lens, photoreceptors, retinal pigment epithelia, visual projections, and electrically active neuronal networks.

Overexpression of signalling molecules is required to define spatial topography of forebrain in organoids. The present invention goes beyond this because organoids according to the invention (e.g. produced according to embodiment 1B) do develop bilaterally symmetric optic vesicles from the forebrain-like region without an artificial induction of signalling centres.

Importantly, these organoids can be generated within 20 to 100 days, 30 to 90 days, 40 to 80 days, 45 to 75 days, 46 to 74 days, 47 to 73 days, 48 to 72 days, 49 to 71 days, or 50 to 70 days. More preferably, these organoids can be generated within 40 to 60 days, 41 to 59 days, 42 to 58 days, 43 to 57 days, 44 to 56 days, 45 to 55 days, 46 to 54 days, 47 to 53 days, 48 to 52 days, or 49 to 51 days. Most preferred is 50 days, which is a time frame that parallels with embryonic retina development and feasible to conduct multiple in vitro experiments. Together, the present invention demonstrates that these engineered brain organoids have the surprising ability to assemble brain-associated primitive sensory organs in a topographically restricted manner allowing interorgan interaction studies within a single organoid. Therefore, the present invention provides significant advances to the field of stem cell-based technologies, tissue engineering, and therapy. Cell counting is performed as specified in the definitions.

### Embodiment 2B

2B. The method according to embodiment 1B, wherein the at least one stem cell is a pluripotent stem cell, an embryonic stem cell, an adult stem cell, a neural stem cell or an embryonic germ cell.

### Embodiment 3B

3B. The method according to embodiment 1B or 2B, wherein the at least one stem cell is selected from the group comprising an induced pluripotent stem cell (iPSC), an embryonic stem cell (ESC), a very small embryonic-like stem cell (VSEL), an amniotic fluid stem cell (AFSC), a marrow-isolated adult multilineage inducible cell (MIAMI), a multipotent adult precursor cell (MAPC) or an unrestricted somatic stem cell (USSC).

### Embodiment 4B

4B. The method according to any one of embodiments 1B to 3B, wherein the at least one stem cell is derived from a human. Preferably, the at least one stem cell is derived from an HLA-homozygous human which is suitable for wide group of human patients.

### Embodiment 5B

5B. The method according to any one of embodiments 1B to 4B wherein the neurospheres are cultured in a composition comprising insulin, a TGFβ/activin/nodal pathway inhibitor and at least one antibiotic. In an alternative embodiment, neurospheres are cultured as before with the exception that no antibiotic is present in order to comply with GMP for later clinical trials.

In a preferred embodiment, neurospheres are cultured in a composition further comprising Pen/Strep and β-Mercaptoethanol. In a more preferred embodiment, neurospheres are cultured in a composition comprising:

TABLE 5

| “Neurosphere | | |
|---|---|---|
| medium”: | | |
| Component: | Final conc. | |
| DMEM/F12 | 48.4% (v/v) | |
| Neural basal medium | 48.4% (v/v) | |
| N2 | 0.4× | |
| B27 w/o Vitamin A | 0.2× | |
| Glutamax | 1× | |
| MEM | 0.5× | |
| Insulin | 0.2755 | μM |
| SB431542 | 2.5 | μM |
| Pen/Strep | 100 | U/ml |
| β-Mercaptoethanol | 5 | μM |

In a preferred embodiment, neurospheres are exposed to a composition comprising i) at least one retinoic acid receptor (RAR) activator, at least one bone morphogenetic protein (BMP) pathway inhibitor and at least one transforming growth factor (TGF) β/activin/nodal pathway inhibitor and Pen/Strep and/or β-Mercaptoethanol. In a more preferred embodiment, neurospheres are exposed to a composition comprising:

TABLE 6

| “Optic vesicle 3D | | |
|---|---|---|
| brain organoid | | |
| medium”: | | |
| Component: | Final conc. | |
| DMEM/F12 | 48.4% (v/v) | |
| Neural basal medium | 48.4% (v/v) | |
| N2 | 0.4× | |
| B27 + RA | 0.2× incl. 60 nMol | |
| | retinol acetate | |
| Glutamax | 1× | |
| MEM | 0.5× | |
| Dorsomorphin | 0.5 | μM |
| Insulin | 0.2755 | μM |
| SB431542 | 5 | μM |
| Pen/Strep | 100 | U/ml |
| β-Mercaptoethanol | 5 | μM |

Using the above-identified method of any of embodiments 1B to embodiment 2B, a 3D brain organoid containing retinal pigment epithelium (RPE) can be generated. For this purpose, the exposure of embodiment 1B is followed by a step wherein the 3D brain organoid is cultured in a medium comprising FBS and/or Pen/Strep and/or β-Mercaptoethanol. In a preferred embodiment, the following medium is used:

TABLE 7

| “RPE medium”: | |
|---|---|
| Component: | Final conc. |
| DMEM/F12 | 87% (v/v) |
| FBS | 10% (v/v) |
| Glutamax | 1× |
| MEM | 1× |
| Pen/Strep | 100 U/ml and 100 μg/ml |
| β-Mercaptoethanol | 5 μM |

DMEM/F12 is a commercially available proprietary basal medium containing essential amino acids, glucose and vitamins. Whenever mentioned in this disclosure, it can be replaced by a basal medium containing essential amino acids, glucose and vitamins.

For later use in clinical trials the GMP compliant “Stem Brew”-medium from Miltenyi could be used.

Neurobasal Medium is a commercially available proprietary basal medium designed for long-term maintenance and maturation of pure pre-natal and embryonic neuronal cells. Whenever mentioned in this disclosure, it can be replaced by a basal medium designed for long-term maintenance and maturation of pure pre-natal and embryonic neuronal cells.

FBS stands for Fetal bovine serum. Whenever mentioned in this disclosure, it can be replaced by fetal serum of another species such as sheep, donkey, horse, pig, cat or dog.

Glutamax is an alternative substrate for L-Glutamate. Whenever mentioned in this disclosure, it can be replaced by L-Glutamate.

### Embodiment 68

6B. The method according to any one of embodiments 1B to 5B, wherein said at least one retinoic acid receptor (RAR) activator is retinol acetate or a derivative thereof, or a semi-synthetic or a synthetic analogue of retinol acetate, or a derivative of such an analogue.

### Embodiment 78

7B. The method according to any one of embodiments 1B to 6B, wherein said at least one retinoic acid receptor (RAR) activator is used in a concentration of greater than 0 and less or equal to 450 nM. In a preferred embodiment, the RAR concentration is between 5 and 430 nM, 10 and 410 nM, 15 and 400 nM, or 20 and 390 nM.

More preferably, the RAR concentration is between 2.5 and 200 nM, 5 and 190 nM, 7.5 and 180 nM, 10 and 170 nM, 12.5 and 160 nM, 15 and 150 nM, 17.5 and 140 nM, 20 and 130 nM, 22.5 and 120 nM, 25 and 110 nM, 27.5 and 100 nM, 30 and 90 nM, 32.5 and 80 nM, or 35 and 70 nM.

Preferred RAR concentrations are, for example: 5, 10, 15, 20, 25, 30, 35, 40, 45, 50, 55, 60, 65, 70, 75, 80, 85, 90, 95, 100, 105, 110, 115, or 120 nM. A concentration of more than 30 and less than 90 nM is most preferred, in particular more than 40 and less than 80 nM. A concentration between 50 and 70 nM is even more preferred, e.g. 51 to 69 nM, 52 to 68 nM, 53 to 67 nM, 54 to 66 nM, 55 to 65 nM, 56 to 64 nM, 57 to 63 nM, 58 to 62 nM, 59 to 61 nM. A RAR concentration of 60 nM is most preferred.

For example, the following results can be achieved when cells of the cell line IMR90 are exposed to retinoic acid:

TABLE 8

| Retinoic acetate | Percentage of organoids |
|---|---|
| (nM) | with optic vesicles (%) |
| 0 | Less than 1 |
| 40 | 21 |
| 60 | 80 |
| 100 | 15 |
| 150 | 10 |
| 200 | 10 |
| 250 | 8 |

### Embodiment 8B

8B. The method according to any one of embodiments 1B to 7B, wherein said at least one BMP pathway inhibitor is dorsomorphin or a derivative thereof, or a semi-synthetic or a synthetic analogue of dorsomorphin, or a derivative of such an analogue.

### Embodiment 9B

9B. The method according to any one of embodiments 1B to 8B, wherein said at least one BMP pathway inhibitor is used in a concentration of 0.05 to 5 μM.

### Embodiment 10B

10B. The method according to any one of embodiments 1B to 9B, wherein said at least one TGFβ/activin/nodal pathway inhibitor is SB431542 or a derivative thereof, or a semi-synthetic or a synthetic analogue of SB431542, or a derivative of such an analogue.

### Embodiment 11B

11B. The method according to any one of embodiments 1B to 10B wherein said at least one TGFβ/activin/nodal pathway inhibitor is used in a concentration of 0.5 to 50 μM.

### Embodiment 12B

12B. The method according to any one of embodiments 1B to 11B, wherein said neurospheres are exposed to a composition comprising 0.05 to 5 pM dorsomorphin, 0.05 to 1.5 μM insulin, 0.5 to 50 μM SB431542, 100 U/ml Pen/Strep, and 0.5 to 5 μM β-Mercaptoethanol.

### C. Product-by-Process Embodiments

### I. Organoid Embodiments

### Embodiment 1C

10. 3D brain organoids obtained by the method of any one of embodiments 1B to 12B.

### II. Tissue (RPE) Embodiments

### Embodiment 2C

2C. Tissue isolated from a 3D brain organoid obtained by the method of any one of embodiments 1B to 12B.

### Embodiment 3C

3C. Tissue according to embodiment 2C, wherein the tissue is a monolayer.

### Embodiment 4C

4C. Tissue according to embodiment 2C or 3C, wherein the tissue comprises cells that are characterized by:
predominantly apical PEDF and basal BEST expression, and/or
PMEL17 expression, and/or
ZO1 expression, and/or
Absence of Ki67 expression, and/or
Expression of cellular retinaldehyde binding protein (CRALBP), and/or
Expression of MITF, and/or
Expression of OTX2, and/or
Secretion of PEDF, and/or
Cobblestone morphology, and/or
an ultrastructure comprising tight junctions marked by phalloidin, basal infoldings, apical microvilli, and melanin granules (Pigments).

### Embodiment 5C

5C. Tissue according to embodiment 4C, wherein the tissue is an artificial retinal pigment epithelium (RPE).

### Embodiment 6C

6C. Tissue according to embodiment 5C which is cryopreserved as an intermediate cell bank.

### Embodiment 7C

7C. Patch comprising an artificial RPE according to embodiment 5C and a synthetic basement membrane or a tissue according to any one of embodiments 2C to 4C and a synthetic basement membrane.

In alternative embodiment (Eₐₗₜ) the basement membrane is not synthetic but rather made of extracellular matrix. The following embodiments 8C to 11C apply to the alternative embodiment (Eₐₗₜ) in analogy.

### Embodiment 8C

8C. Patch according to embodiment 7C, wherein the synthetic basement membrane comprises a polyester membrane and/or parylene C.

### Embodiment 9C

9C. Patch according to embodiment 8C, wherein the synthetic basement membrane comprises a polyethylene terephthalate (PET) membrane, optionally coated in plasma-derived vitronectin.

### Embodiment 10C

10C. Patch according to embodiment 7C, wherein the synthetic basement membrane is biodegradable.

### Embodiment 11C

11C. Patch according to any one of embodiments 7C to 10C, wherein the synthetic basement membrane comprises a scaffold manufactured using poly(lactic-co-glycolic acid) (PLGA), preferably (50:50 lactic acid/glycolic acid, four midpoint 1.0 dl/g), with 350 nm mean fiber diameter.

Explanations Concerning Synthetic Basement Membrane (Embodiments 7C to 11C)

Sharma et al. (Clinical-grade stem cell-derived retinal pigment epithelium patch rescues retinal degeneration in rodents and pigs. Sci Transl Med. 2019 Jan. 16; 11(475)) demonstrated that a clinical-grade AMD iRPE patch safely integrates in the eye and shows improved efficacy over cell suspension in a rodent preclinical study. They also showed that human clinical dose of the AMD iRPE patch integrates in the eye of a laser-induced RPE injury pig model and rescues degenerating retina.

Without being intended to be bound by theory, it is believed that a biodegradable scaffold provides suitable material for RPE cells or other tissue according to the invention to secrete extracellular matrix (ECM) to form a polarized monolayer. If the scaffold degrades, ECM and cells would constitute a native-like (RPE) tissue that would enhance the possibility of long-term integration of the (iRPE) patch in the host (e.g. the patients' eyes).

### D. Pharmaceutical Composition Embodiments

### Embodiment 1D

1D. A pharmaceutically acceptable composition comprising the (optic vesicle) 3D brain organoid according to any one of embodiments 1A to 11A or obtained by the method of any one of embodiments 1B to 12B and/or tissues and/or cells and/or molecules derived therefrom.

### E. Medical Use Embodiments

### Embodiment 1E

1E. A pharmaceutically composition according to embodiment 1D for use in the treatment of diseases associated with visual impairment including retinal disorders. Preferably, the pharmaceutical composition is GMP/ATMP-acceptable.

Alternatively, 3D brain organoids according to any one of claims 1 to 11A or obtained by the method of any one of embodiments 1B to 12B for use in the treatment of diseases associated with visual impairment including retinal disorders.

### Embodiment 2E

2E. A pharmaceutically acceptable composition according to embodiment 1E for use in the treatment of diseases selected from age-related macular degeneration (AMD), retinitis pigmentosa (RP), Leber congenital amaurosis (LCA), Stargardt disease, choroideremia, Usher syndrome, X-linked retinoschisis (XLRS), achromatopsia (ACHM), and glaucoma. Preferably, the pharmaceutical composition is GMP/ATMP-acceptable.

### Embodiment 3E

3E. Tissue according to any one of embodiments 3C to 5C or patch according to any one of embodiments 6C to 9C for use in the treatment of diseases associated with visual impairment. Preferably, the tissue or patch is produced under GMP/ATMP compliance.

### Embodiment 4E

4E. Tissue or patch for use in the treatment of diseases associated with visual impairment according to embodiment 3E, wherein the disease is selected from age-related macular degeneration (AMD), retinitis pigmentosa (RP), Leber congenital amaurosis (LCA), Stargardt disease, choroideremia, Usher syndrome, X-linked retinoschisis (XLRS), achromatopsia (ACHM), and glaucoma. Preferably, the tissue or patch is produced under GMP/ATMP compliance.

### Embodiment 5E

5E. Tissue or patch for use in the treatment of diseases associated with visual impairment according to embodiment 3E or 4E, wherein tissue or patch is transplanted into the eye of a patient, preferably wherein the transplantation is preceded and/or followed by immunosuppression. Preferably, the tissue or patch is produced under GMP/ATMP compliance.

### Embodiment 6E

6E. Tissue or patch for use in transplantation according to embodiment 5E, wherein immunosuppression consists of perioperative prednisolone and long-term intraocular steroid implants. Preferably, the tissue or patch is produced under GMP/ATMP compliance.

### Embodiment 7E

7E. Tissue or patch for use in the treatment of diseases associated with visual impairment according to embodiment 3E, wherein transplantation is followed by subretinal administration of a Rho kinase inhibitor and/or a ROCK inhibitor such as Y27632. Preferably, the tissue or patch is under GMP/ATMP compliance.

### F. Embodiments for Use

### Embodiment 1F

1F. Use of the optic vesicle 3D brain organoid according to embodiments 1A to 10A in toxicity in vitro assays including bacterial and viral infection tests.

### Embodiment 2F

2F. Use of the optic vesicle 3D brain organoid according to embodiment 1F to 10F in modelling retinopathies and eye development.

### Embodiment 3F

3F. Use of the optic vesicle 3D brain organoid according to embodiment 1F to 10F in drug screening in vitro assays.

### G. Composition Embodiments

### Embodiment 1G

1G. Composition comprising at least one retinoic acid receptor (RAR) activator, at least one bone morphogenetic protein (BMP) pathway inhibitor and at least one transforming growth factor (TGF) β/activin/nodal pathway inhibitor.

### Embodiment 2G

2G. Composition according to embodiment 1G, wherein said at least one retinoic acid receptor (RAR) activator is retinol acetate or a derivative thereof, or a semi-synthetic or a synthetic analogue of retinol acetate, or a derivative of such an analogue.

### Embodiment 3G

3G. Composition according to embodiment 1G or 2G, wherein said at least one retinoic acid receptor (RAR) activator is used in a concentration of greater than 0 and less or equal to 250 nM.

### Embodiment 4G

4G. Composition according to any one of embodiments 1G to 3G, wherein said at least one BMP pathway inhibitor is dorsomorphin or a derivative thereof, or a semi-synthetic or a synthetic analogue of dorsomorphin, or a derivative of such an analogue.

### Embodiment 5G

5G. Composition according to any one of embodiments 1G to 4G, wherein said at least one BMP pathway inhibitor is used in a concentration of 0.05 to 5 μM.

### Embodiment 6G

6G. Composition according to any one of embodiments 1G to 5G, wherein said at least one TGFβ/activin/nodal pathway inhibitor is SB431542 or a derivative thereof, or a semi-synthetic or a synthetic analogue of SB431542, or a derivative of such an analogue.

### Embodiment 7G

7G. Composition according to any one of embodiments 1G to 4G, wherein said at least one TGFβ/activin/nodal pathway inhibitor is used in a concentration of 0.5 to 50 μM.

### Embodiment 8G

8G. Composition according to any one of embodiments 1G to 7G, wherein the composition comprises 0.05 to 5 μM dorsomorphin, 0.05 to 1.5 μM insulin, 0.5 to 50 μM SB431542, 100 U/ml Pen/Strep, and 0.5 to 5 μM β-Mercaptoethanol.

### Embodiment 9G

9G. Use of the composition according to any one of embodiments 1G to 8G for generating a 3D brain organoid according to any one of embodiments 1A to 12A.

### EXAMPLES

Description of the Figures

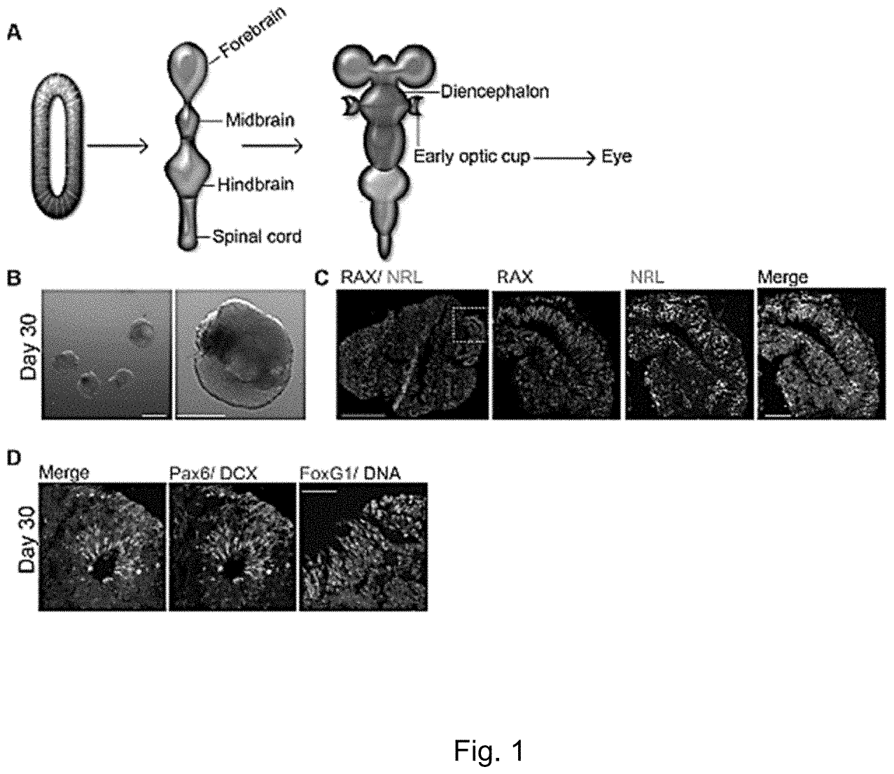

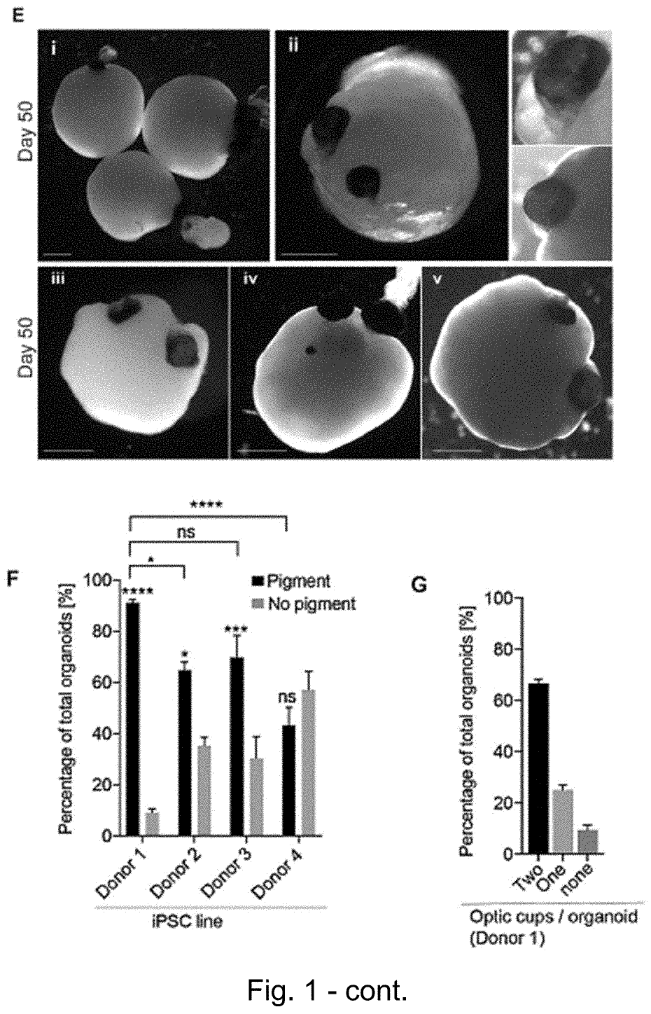

FIG. 1. Generation of Optic Vesicle 3D Brain Organoids from Human iPSCs.

A. Schematic of human embryonic nervous system development. The neural tube (left) segregates into forebrain, midbrain, hindbrain, and spinal cord (middle). Forebrain partially develops into diencephalon from which the early optic vesicles invaginate laterally (right). Optic vesicles later form the eye.

B. 30 day old organoids are displaying the early occurrence of dark pigments (arrows). Scale bar, 1 mm (left panel) 500 μm (right panel). Representative image, cell line F14536.2.

C. Organoids display RAX-positive primordial eye field (magenta) and NRL-positive photoreceptor progenitors (yellow). Scale bar, 500 μm for whole organoid and 50 μm (inset). Representative image, cell line IMR-90. Representative images are shown. N=10 organoids from at least 4 independent batches.

D. 30 day old organoids show forebrain markers Pax6 (green, left and middle) and FoxG1- (green, right) containing ventricular zones. Scale bar, 50 μm. Representative images are shown. N=16 organoids from at least 4 independent batches. Representative image, cell line IMR-90.

E. Macroscopic views of 50-day-old organoids with each containing bilaterally symmetric dark pigmented areas (i-v). Insets of (ii) show close up view of individual pigmented regions. Scale bar, 1 mm. Representative images from at least four independent batches of organoids. Representative images, cell lines IMR-90 (i)+(ii), F13535.1 (iii+v), GM25256 (iv).

F. Bar diagram quantifies organoids with pigmented optic vesicles. On an average, iPSC line of donor 1 yields the highest number organoids with pigmented optic vesicles (91%). Donor 2, 3 and 4 yield 65%, 70% and 43%, respectively. For each cell line, at least three different batches were generated. Donor 1: number of organoids=95, n=5 batches; donor 2: number of organoids=100, n=4 batches; donor 3: number of organoids=87, n=4 batches; donor 4: number of organoids=32, n=3 batches. Cell lines IMR-90, GM25256, F13535.1, F14536.2. Statistical analysis within and across cell lines. Cell lines IMR-90, GM25256, and F13535.1 produced significantly more brain organoids with pigments than without. Cell line F14536.2 produced a similar number of brain organoids with and without pigment. Two-way ANOVA followed by Sidak's multiple comparisons test. Significance within cell lines: IMR-90: n=5, p****<0.0001; GM25256: n=4, p*<0; F13535.1: n=4, p***<0.001; F14536.1: n=3, ns. Significance across cell lines compared to IMR-90. IMR-90 produced significant higher number of pigmented organoids per batch compared to GM25256, p*<0.1 and F14536.2. There was no significant difference between the number of pigmented organoids produced by IMR-90 and F13535.1 (ns).

G. Bar diagram quantifies percentages of two-, one- or no optic vesicles-containing organoids derived from donor 1 (IMR90) iPSC cells. In an average approx. 66% of organoids differentiated from this cell line yielded two pigmented optic vesicles restricted at one pole. Number of organoids=95, n=5 batches.

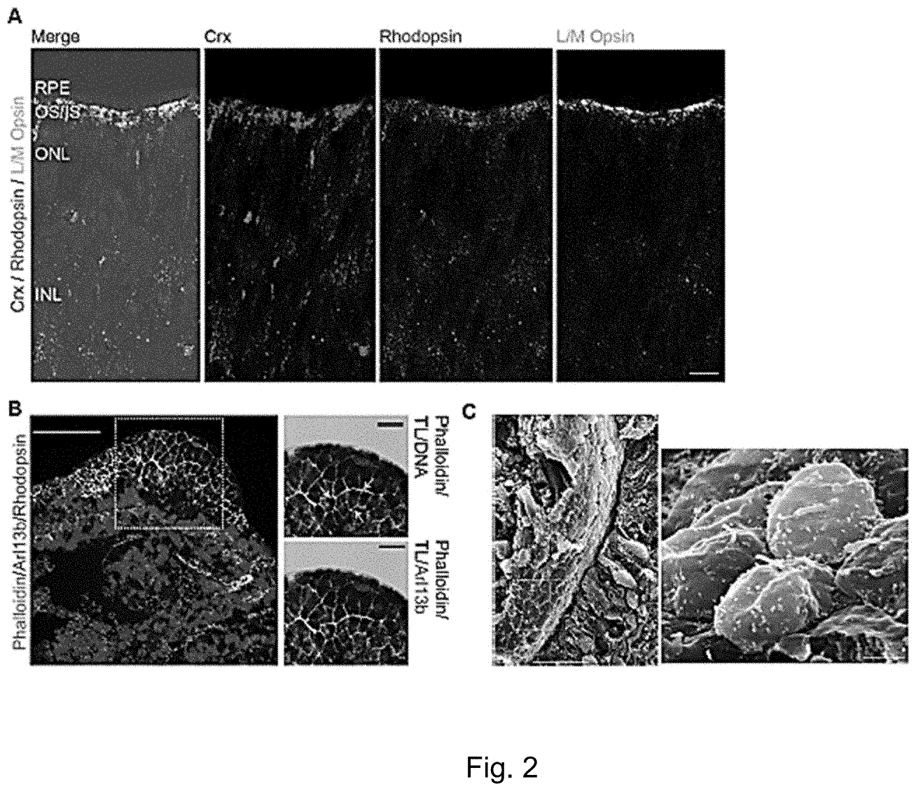

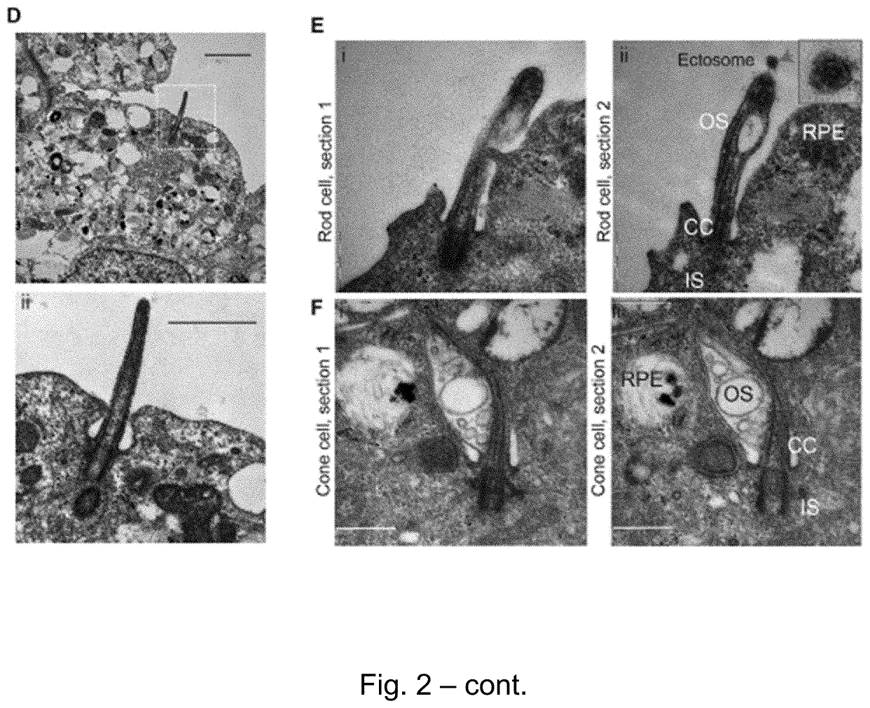

FIG. 2. Optic Vesicle Brain Organoids Display Stratified Neural Retina.

A. Transverse section through an optic vesicle of an organoid shows early tissue stratification beginning from RPE cell layer imaged by transmission light (top) and the photoreceptor cell layer with outer segments (OS) positive for CRX (magenta), Rhodopsin (green) and Opsin (yellow). Below the inner segment (IS) is the outer nuclear layer located (ONL), visualized by nuclear staining (blue). The dense, thick layer of nuclei (bottom) is the inner nuclear layer (INL). Scale bar, 10 μm. N=10 organoids from at least four independent batches. Representative image, cell line GM25256.

B. Section through the RPE cell layer of an organoid. The dotted box marks the pigmented area consisting of honeycomb-shaped RPE cells visualized by Phalloidin (arrows) and primary cilia (Arl13B). Retinal pigment (black) hinders the visualization of nuclei and Rhodopsin. Scale bar (left panel), 50 μm. Scale bar (inset, right panel), 10 μm. N=21 organoids from at least four independent batches. Representative image, cell line IMR-90.

C. Sagittal view of RPE imaged by scanning EM reveals tightly packed single layer RPE cells (pseudocolored). Primary cilium projects from each cell (pseudocolored). Scale bar, 20 μm (left image) and 2 μm (right image). Representative image, cell line IMR-90.

D. Transmission EM of an RPE cell with numerous melanosomes (arrows) and a primary cilium (white square) (i). Magnified primary cilium with a cross-sectioned daughter centriole (ii). At least twenty RPE cells were imaged from two independent batches of organoids. Scale bar, Ei, 2 μm and Eii, 1 μm. Representative images, cell line IMR-90.

E. and F. Serial section of developing photoreceptor cilium (E i-ii). Cilium is emerging from a centriole bulging out in the distal area to form an outer segment (OS) suggestive of a rod cell. The OS is distinguishable from the connecting cilium (CC) at the base. An ectosome (arrow and inset) is released at the tip of the outer segment that takes part in the visual cycle and photoreceptor disc retention and shedding. Right next to the rod cell is an RPE cell with melanosomes. Serial crosssection of a developing photoreceptor with a triangular-shaped outer segment suggestive of a cone cell (F i-ii). The OS bulging out from the base of the cilium contains several vesicles. An RPE cell is neighboring the cone cell characterized by melanosomes in the cytoplasm. Scale bars, 500 μm. At least nine photoreceptor cells were imaged from two independent batches of organoids. Representative images, cell line IMR-90.

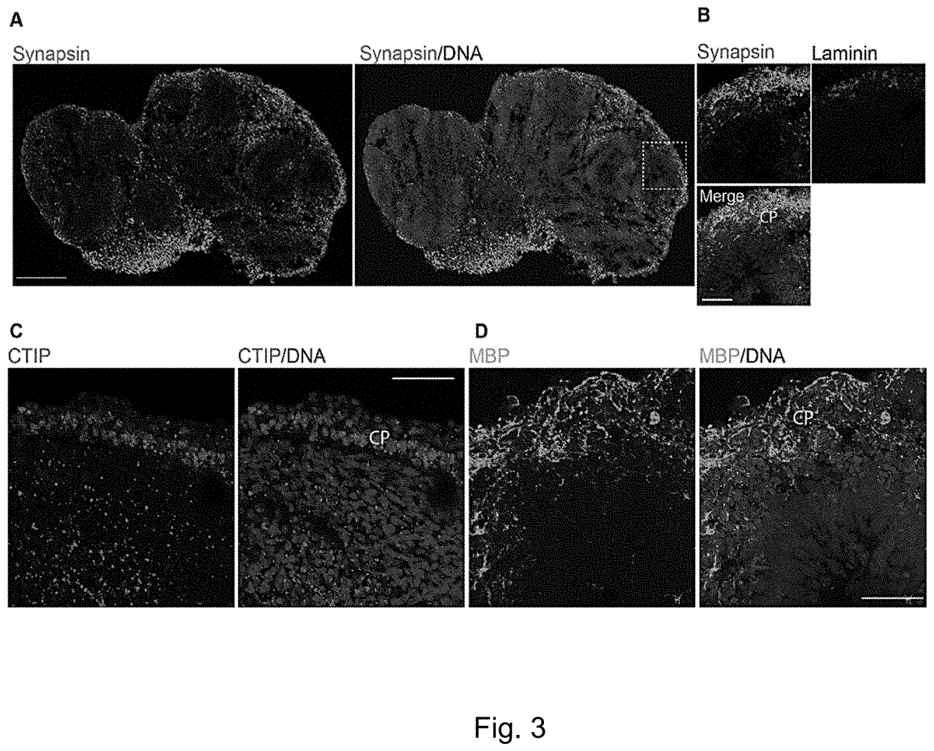

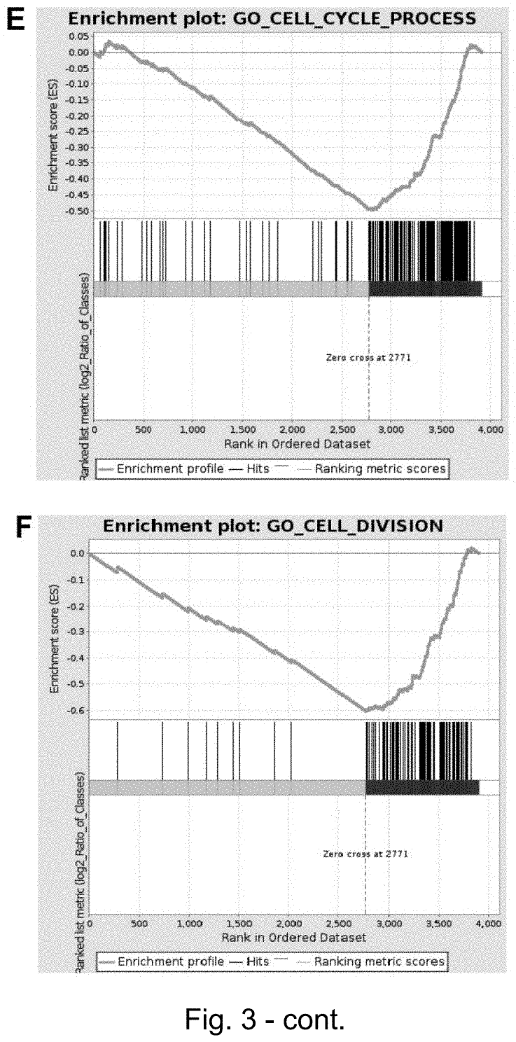

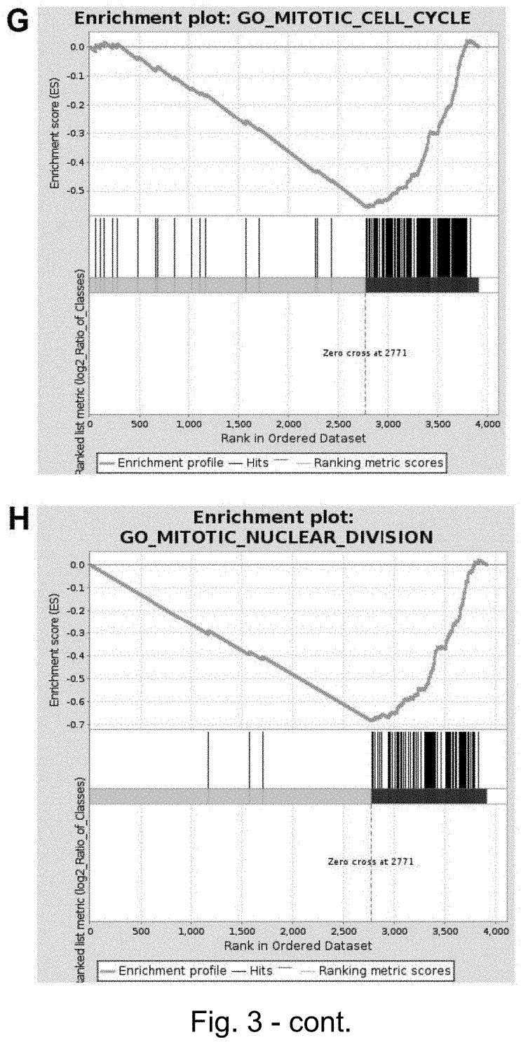

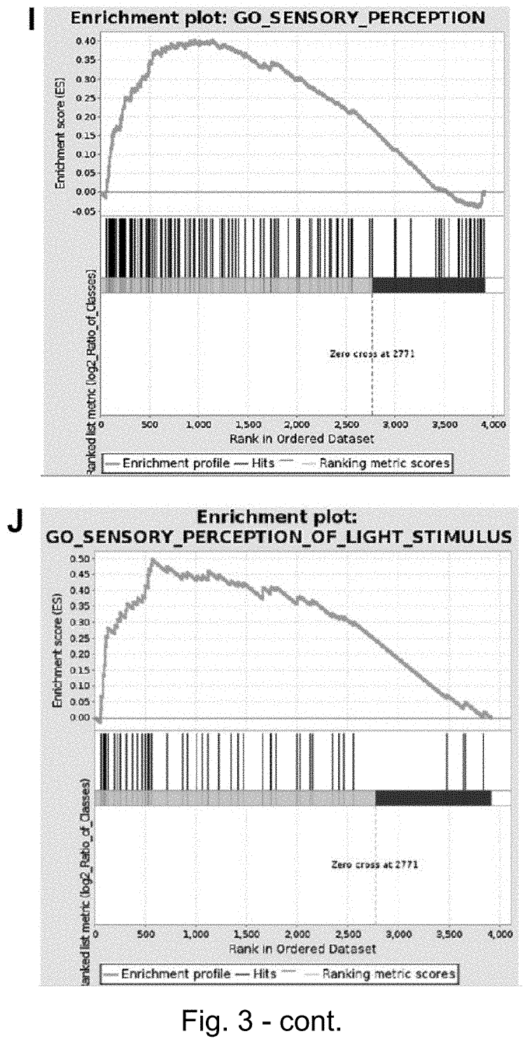

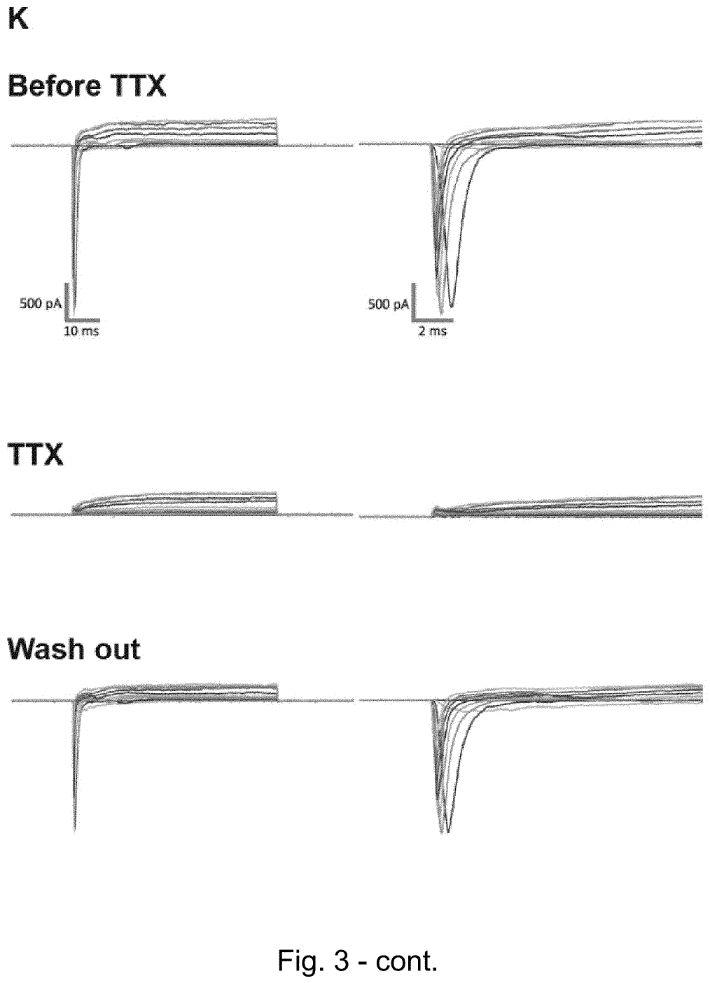

FIG. 3. Optic Vesicle Brain Organoids Display Mature Neurons

A-B. Section of the whole organoid stained for Synapsin and Laminin, proteins essential for the release of synaptic vesicles at the presynaptic terminals, and polarization and layering of the neurons in the cortical region, respectively. CP, cortical plate. Scale bar, 500 μm (overview of the whole organoid) and 50 μm (inset). Representative images are shown. N=17 organoids from at least five independent batches. Representative images, cell line IMR-90.

C-D. Cortical region of a 3D brain organoid exhibits a CTIP2 (neurons of layer V) positive cells layer. Scale bar, 25 μm. MBP (myelin basic protein) positive region indicating the presence of myelin-producing oligodendrocytes (D). Representative images are shown. N=12 organoids from at least four independent batches. Representative images, cell line IMR-90.

E. Enrichment plot showing cell cycle processes down-regulated in optic vesicle brain organoids, indicating higher numbers of matured cell types compared to early brain organoids. Cell line IMR-90.

F. Enrichment plot showing cell cycle division is down regulated in matured optic vesicle brain organoids indicative of a higher number of post-mitotic cells compared to early brain organoids.

G. Enrichment plot showing mitosis genes are down regulated, as shown in panels E-F.

H. Enrichment plot showing optic vesicle brain organoids contain cells that mostly have downregulated the genes for mitotic nuclear division in contrast to fast-cycling progenitor cells in early brain organoids.

I. Enrichment plot showing optic vesicle brain organoids contain cells with up-regulated gene expression for sensory perception.

J. Enrichment plot showing optic vesicle organoids contain cells expressing genes for sensory perception of light stimuli. Results from panels (I) and (J) support the presence of matured functional cell types relevant to eye development in the optic vesicle compared to early brain organoids.

K. Two currents (inward and outward) are seen (about 400 pA at +40 mV). The outward current is TTX insensitive and is an outwardly rectifying K+ current. Whole-cell patch-clamp recording revealed that neurons show a transient inward TTX (Tetrodotoxin) sensitive current. TTX is a neurotoxin that selectively blocks the sodium channel. Here is shown one recording example of a neuron with a high Na+ current. Upper two graphs show the recorded current before treatment. Middle graphs show blocked Na+ current (inward current) by TTX treatment. And bottom panels show reoccurring Na+ current after wash out of TTX. Scaling bar is 500 pA. Total recorded cells were 27 and 36 per organoid from two independent batches. Cell lines IMR-90 and GM25256.

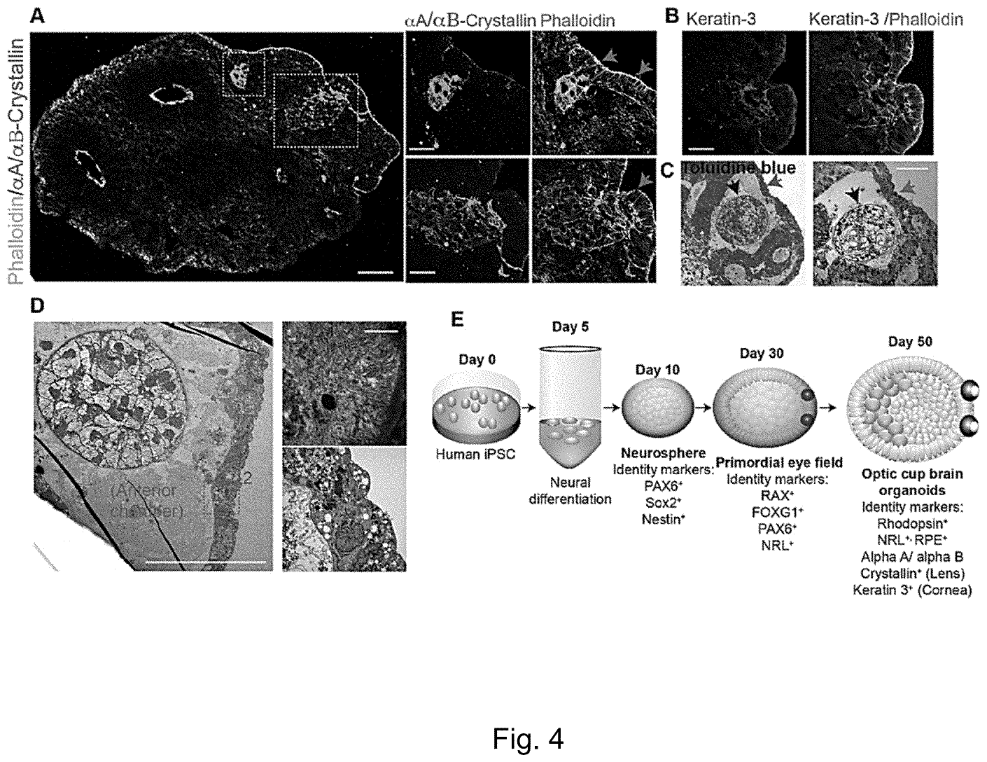

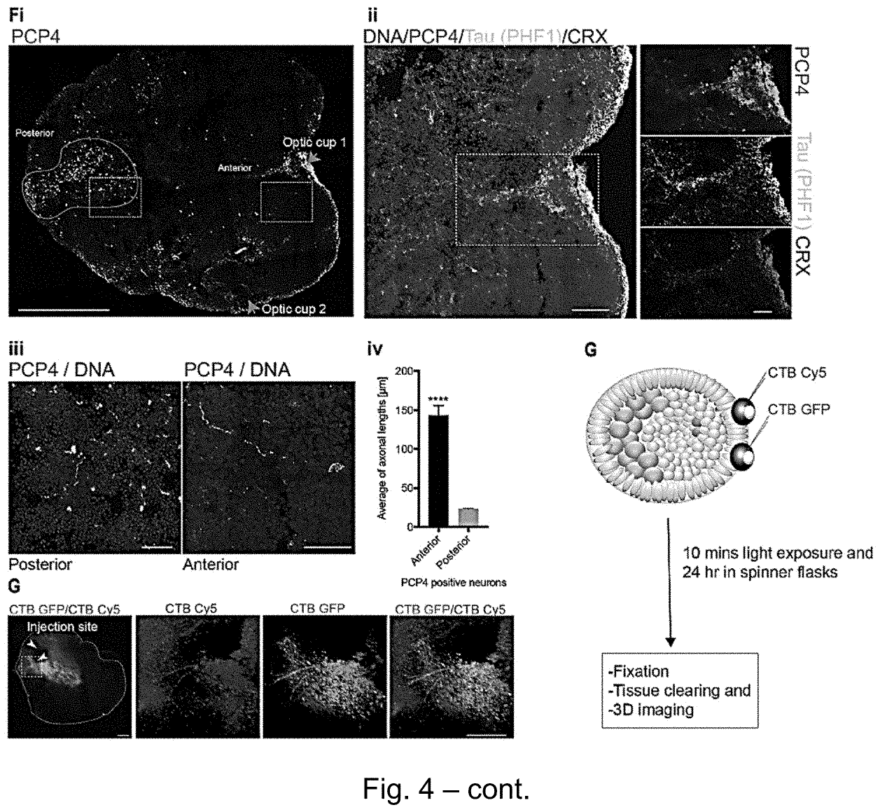

FIG. 4. Optic Vesicle Brain Organoids Display Non-Neuronal Components and Optic Tracts Connected to Brain Organoids

A. 50-day-old optic vesicle 3D brain organoid stained with F-actin marker Phalloidin (yellow and arrows). Two lenses are stained by a lens marker A/B Crystallin. Scale bar, 100 μm. Magnified images are at right. Scale bar, 50 μm. Representative images are shown. N=10 organoids from at least three independent batches. Representative images, cell line IMR-90.

B. Lenses are neighboured by a single epithelial cell layer that is Keratin-3 positive. Representative images are shown. Scale bar, 50 ∞m. N=7 organoids from at least three independent batches. Representative images, cell line IMR-90.

C. Ultrathin section of the optic vesicle region stained with toluidine blue staining showing a well-defined round lens (black arrow) in a cavity. Red arrow points at the cell monolayer of the cornea on the surface next to the lens. Panel at right is an EM image of the similar region with lens (black arrow) and cornea (lighter arrow). Scale bar, 50 μm. Representative image, cell line GM25256.

D. Magnified view of the same lens. Anterior chamber enclosed by the cornea. Square 1 shows an area where the transition from the lens vesicle to lens fibers happened. The lens fibers are visible in inset 1 (upper right panel). Inset 2 (lower right panel) shows magnified corneal cell layer with typical tight junctions. Cornea is separated by a basal membrane (probably early Descemet's membrane) from the inner chamber. Scale bar left image, 100 μm. Scale bar insets, 10 μm.

E. Schematic of optic vesicle brain organoids generation. In the course of differentiation, iPS cells form neural epithelium (markers: PAX6, SOX2, Nestin) and forebrain-like region with eye fields (markers: FoxG1, RAX, PAX6, NRL) in order to finally develop into a 3D brain organoidwith optic vesicles consisting of retina, lens, and cornea.

F. (i) Whole organoid section stained for PCP4, which is in vivo expressed in the retina, developing lateral geniculate nucleus (LGN) and visual cortex (area 17). PCP4 signals in the optic vesicle region (arrow, optic vesicle 1), whose axons merge to a possible optic stalk (the precursor of the optic nerve), and in cells of the posterior region of the organoid (circled area). Scale bar, 500 μm. ii. Right images show magnified optic vesicle 1 area with CRX and PCP4-positive retinal region. Bar, 100 μm for merged image and 50 μm for insets. Magnified panels at right show PCP4 region merges with optic stalk where axons are PHF1 (adult form of Tau) positive (yellow). Scale bar, 100 μm for merged image and 50 μm for insets. iii. The panel shows magnification of squares in the anterior and posterior region of the organoid with neurons positive for PCP4 (green). Scale bar, 50 μm. iv. Quantification of axonal lengths of PCP4 positive neurons in the anterior and posterior region revealed that anterior PCP4 positive neurons possess significantly longer axons than those of the posterior region. Axons of 100 neurons from 3 different batches of organoids were measured. Unpaired t-test One-way ANOVA, p****<0.0001, n=3. Error bars show +/−SEM. Representative image, cell line IMR-90.

G. Experimental scheme of CTB injection experiment. Lower panel: Representative volume-rendered images of optic vesicle organoids injected with Cholera Toxin B (CTB-488 and CTB-647) at two different optic vesicles of the same organoid. White arrowheads mark the site of injection. Reconstructed images of individual channels, as well as merged channels, are shown at right. Scale bars, 200 μm. N=13 organoids from 4 independent batches. Cell line IMR-90.

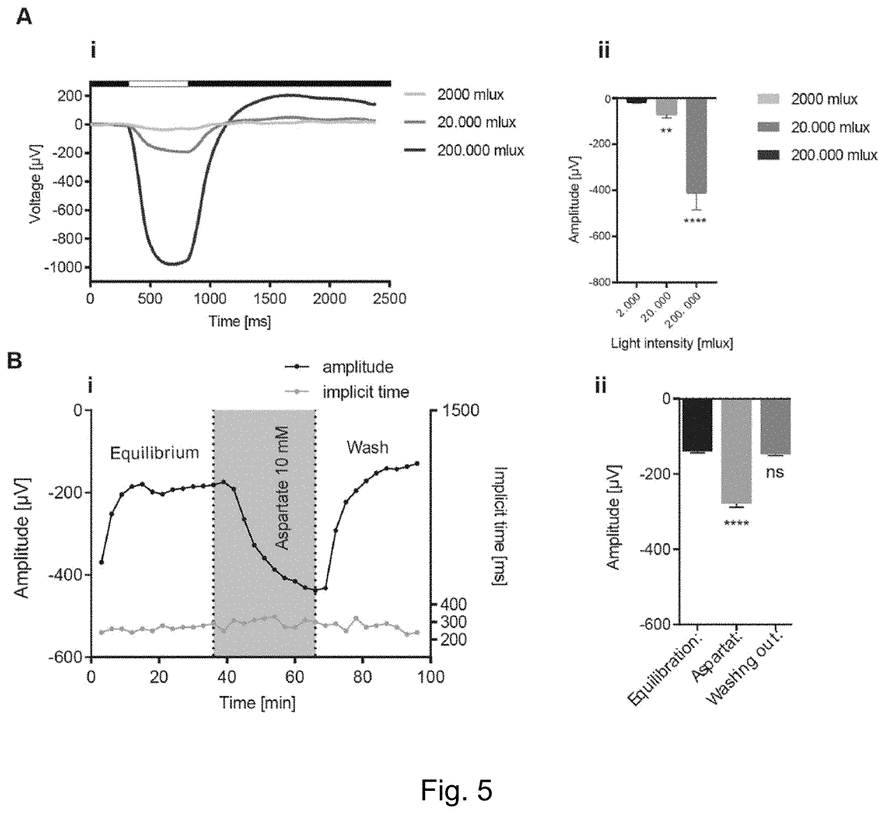

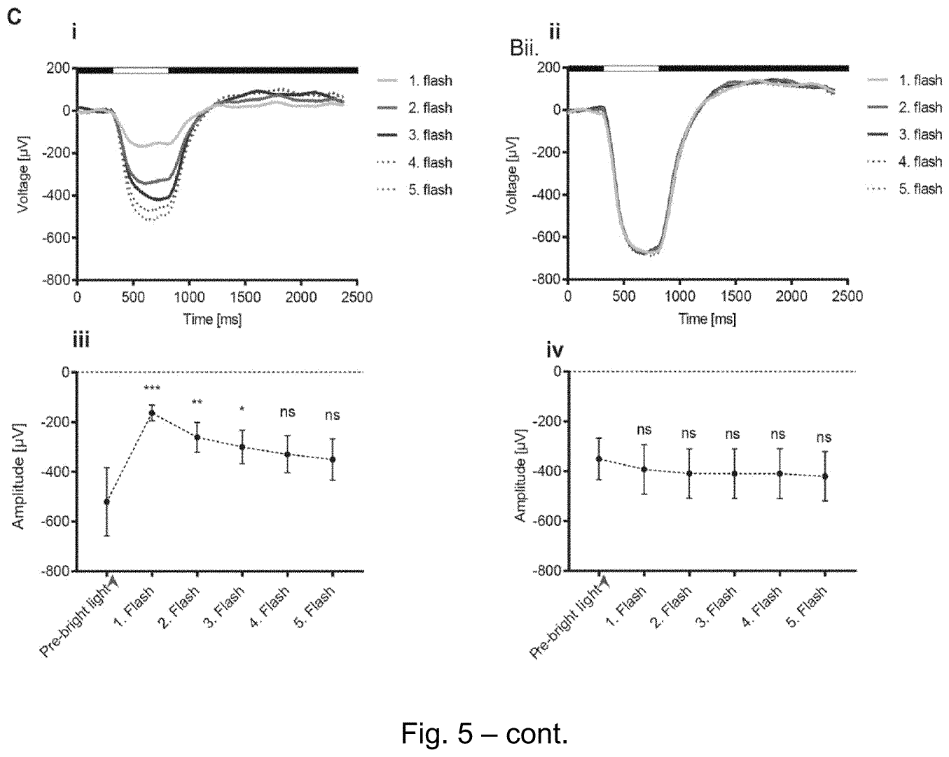

FIG. 5 Optic Vesicle Brain Organoids are Light Sensitive.

A. (i). A dose-dependent light response stimulated with three different light intensities. The white line at the top of the graph marks the time span of light flash (500 ms). ii. Statistical summary. Each organoid was exposed and recorded every 3 minutes for 3× 500 ms to 2000 mlux (candle light), then 3× 500 ms to 20.000 mlux (˜street light at night) and finally 3× 500 ms to 200.000 mlux (˜sunset or sunrise on a clear day). Light response of organoids increased significantly with increasing light intensity (20.000 mlux compared to 2.000 mlux p<0.001, 200.000 mlux compared to 2.000 mlux p<0.00001). Kruskal-Wallis test of One-way ANOVA data: non-parametric; Dunn's multiple comparisons test; mean with error bars SEM; n=9. Cell line IMR-90.

B. Isolating A-wave within the retinal signaling network. i. ERG upon aspartate treatment. During equilibration time of 36 min, organoid got adjusted to the system under 2 ml/min superfusion and the light flash of 500 ms with 200.000 mlux light intensity until it showed the stable amplitude of −200 pV. Within 6 min of aspartate application, optic vesicle 3D brain organoidshowed a drop in amplitude up to lesser than −400 pV upon light flashes. After aspartate washout, organoid shows amplitude that is similar to aspartate-free condition. ii. Statistical summary of aspartate treatment. 10 mM aspartate treatment led to a significant hyperpolarization of −278 pV on average compared to equilibration before aspartate treatment (−140 pV on average) (****p<0.0001). Hyperpolarization after washout of aspartate is similar to value before aspartate application (−147 pV). Error bars show mean+/−SEM. One-way ANOVA data followed by Dunnett test. N=3 organoids. Cell line GM25256.

C. Photosensitivity of optic vesicle organoids can be desensitized by bright light exposure with the light intensity of 4600 Lux for 10 minutes (i). White line at the top of the graph marks the time span of light flash (500 ms). (ii). Negative control experiment. The same organoid was treated after 30 min recovery from photic stress under the same conditions in which bright light exposure was replaced by 0 lux. iii. Statistical summary of photic stress experiments. Optic vesicle organoids in electrode chamber were adapted for 15 min to the ERG system (Pre-bright light) with light flashes (200.000 mlux) and recording every 3 minutes. Then organoids were photically stressed by exposure to 4600 lux for 10 minutes (arrow). While average amplitude before bright light exposure was −521 pV, photic stress led to a significant reduction of photosensitivity (p<0.0001 after 30 s of recovery, p<0.001 after 3:30 min of recovery and p<0.01 after 6:30 min of recovery). Finally, there was no significant difference in light response detectable after 9:30 min and 12:30 min of regeneration suggesting a complete recovery from photic stress within this time period. Friedman test of One-way ANOVA data: non-parametric; Uncorrected Dunn's test; mean with error bars SEM; n=4. iv. Negative control experiment. After recovery from photic stress, each organoid was exposed to the same treatment in which the bright light was replaced by 0 lux (arrow). No significant difference was detected in photosensitivity, suggesting that the response detected in graph C was solely due to bright light exposure. Friedman test of One-way ANOVA data: nonparametric; Uncorrected Dunn's test; mean with error bars SEM; n=4. Organoids were superfused in organoid medium with a speed of 0.5 ml/min.

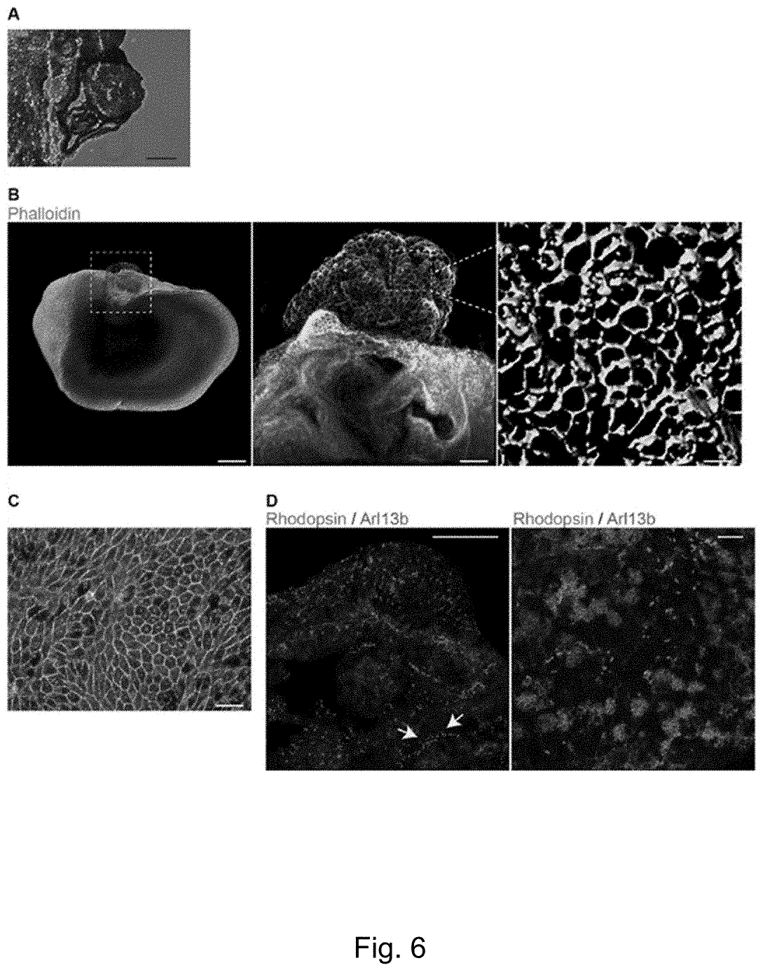

FIG. 6. Related to FIG. 2.

A. Section through an optic vesicle region stained with hematoxylin showing stratified layers of neural retina. Inner cell layer is darkly pigmented, which could be a RPE of a retina. Scale bar, 50 μm. Representative image, cell line F14536.2.

B. Surface rendered image of an optic vesicle 3D brain organoid stained with Phalloidin. The square at the apex (left panel) highlights the optic vesicle, which is magnified in the middle panel. The right panel is a 3D construction of the RPE within the optic vesicle. Scale bars, 200 μm (left panel), 50 μm (middle panel), 5 μm (right panel). Phalloidin failed to penetrate the inner core of the organoid (left panel). Representative image, cell line GM25256.

C. Adherent RPE cells dissociated from an optic vesicle form a monolayer RPE sheet displaying typical honeycomb shaped cells filled with pigments. Scale bar, 10 μm. Representative image, cell line IMR-90.

D. Immunostaining through an optic vesicle shows Arl13B-positive primary cilia in the pigmented RPE cells (Upper region). Note that pigmented region strongly hinders the visualization of Rhodopsin immunoreactivity. Serially organized primary cilia in a region of Rhodopsin-positive cells below the pigmented cells (arrows). At right, another example with a magnified view. Scale bar, 50 μm. Right image shows a representative image of a second organoid. Scale bar 10 μm. N=11 from 3 independent batches. Representative image, cell line IMR-90.

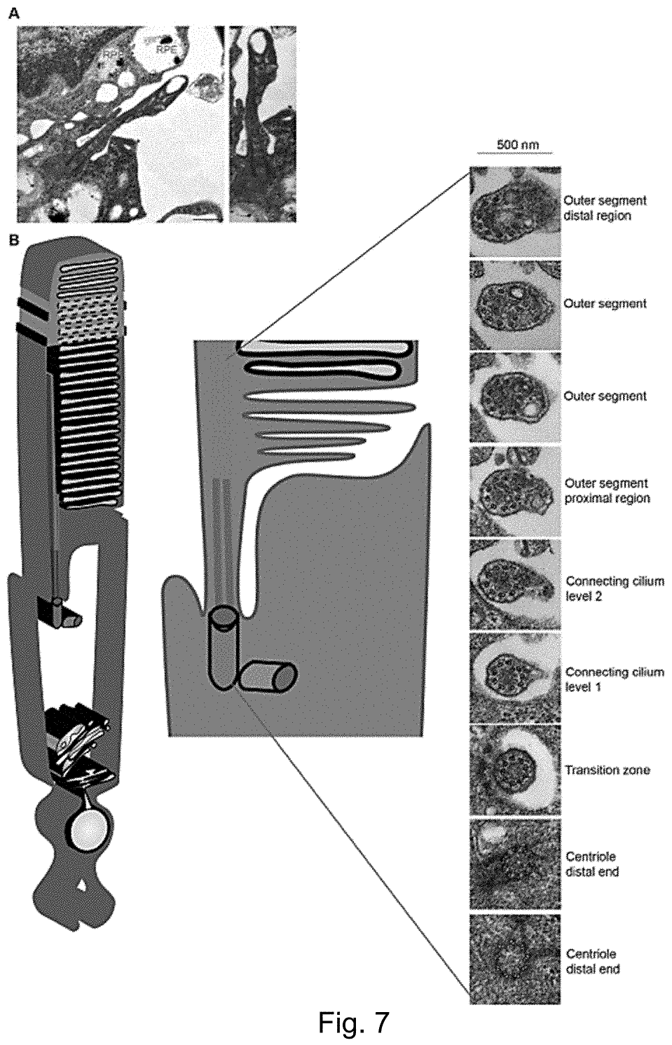

FIG. 7. Related to FIG. 2.

A. Transmission EM image of a developing photoreceptor rod cell. Left panel: The outer segment (OS) is bulging out as an extension at the tip of the connecting cilium (CC). The inner segment (IS) is partly shown and contains the cell body. The photoreceptor cell is neighbored by a retinal pigment epithelial (RPE) cell containing dark pigment granules. Right panel: The outer segment (OS) is filled with vesicles that will later fuse to OS discs. The outer segment appears to develop through an enlargement of the distal part. These longitudinal serial sections additionally showed the process of vesicle formation restricted to only one side of the cilium that is opposite to the side where the axoneme is located. This type of asymmetrical development is a key feature of outer segment differentiation in rod cells of rats 51-53 and cat54. Scale bar, 500 nm. Representative images, cell line IMR-90.

B. Serial cross-sections of a developing rod outer segment. The schematic on the left shows a rod cell from the inner segment with nucleus, centrioles, connecting cilium and outer segment with discs for photoreception. Middle schematic shows the magnified region from centrioles to outer segment distal region in the longitudinal section for orientation. The right panel shows serial cross-sections through the entire length of the connecting cilium and outer segment. These cross-sections identify that membranous vesicles are indeed asymmetrically distributed to only one side of the cilium. At the bottom, a typical basal body consisting of triplet microtubules with appendages and Y-linkers is seen. At the distal part of the connecting cilium, a small bulge appeared lateral to the axoneme, which continued to grow more significant in the successive sections. More distally, numerous vesicles and flattened cisternae were found within the lateral expansion resembling the pattern of outer segment formation. Scale bar, 500 nm. Representative images, cell line IMR-90.

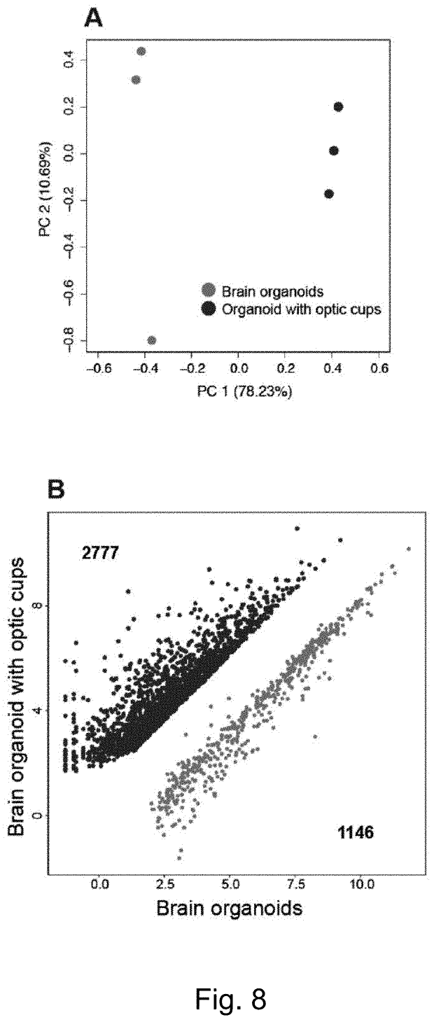

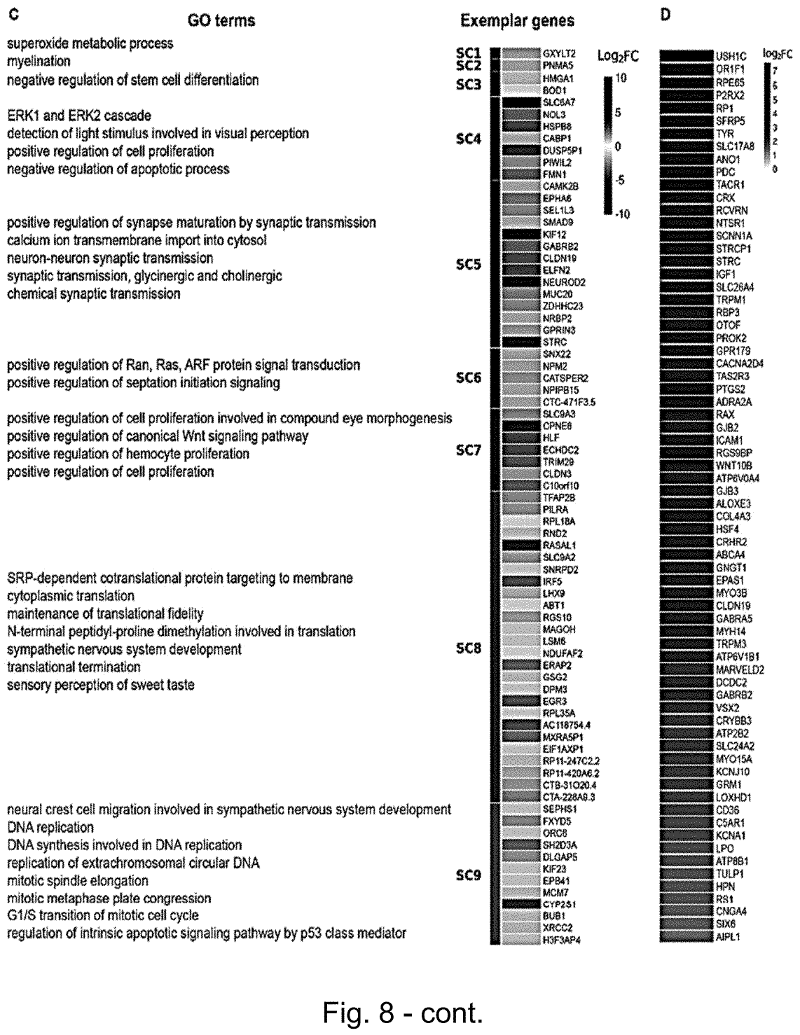

FIG. 8

A. Analysis of mRNA-seq data from early brain organoids and optic vesicle brain organoids (both generated from cell line IMR-90). Principal component analysis biplot of mRNA-seq data derived from brain organoids and optic vesicle brain organoids. PC1 contributes to ˜80% of the variance between the sample subgroups.

B. Scatter plot of normalized counts from mRNA-seq data reveals 2777 genes being pregulated and 1146 down-regulated in optic vesicle brain organoids compared to brain organoids.

C. Affinity propagation clustering results represented by the log2 fold-changes of exemplary genes from 9 super-clusters and their respective gene ontology terms indicating super-clusters 4 and 7 as clusters with genes involved in visual perception and eye morphogenesis.

D. Leading-edge analysis of GSEA results for super-cluster 4 reveals a retinal development gene signature enrichment, exemplified by retinal progenitor and ganglion markers, as well as typical photoreceptor cells transcription factors.

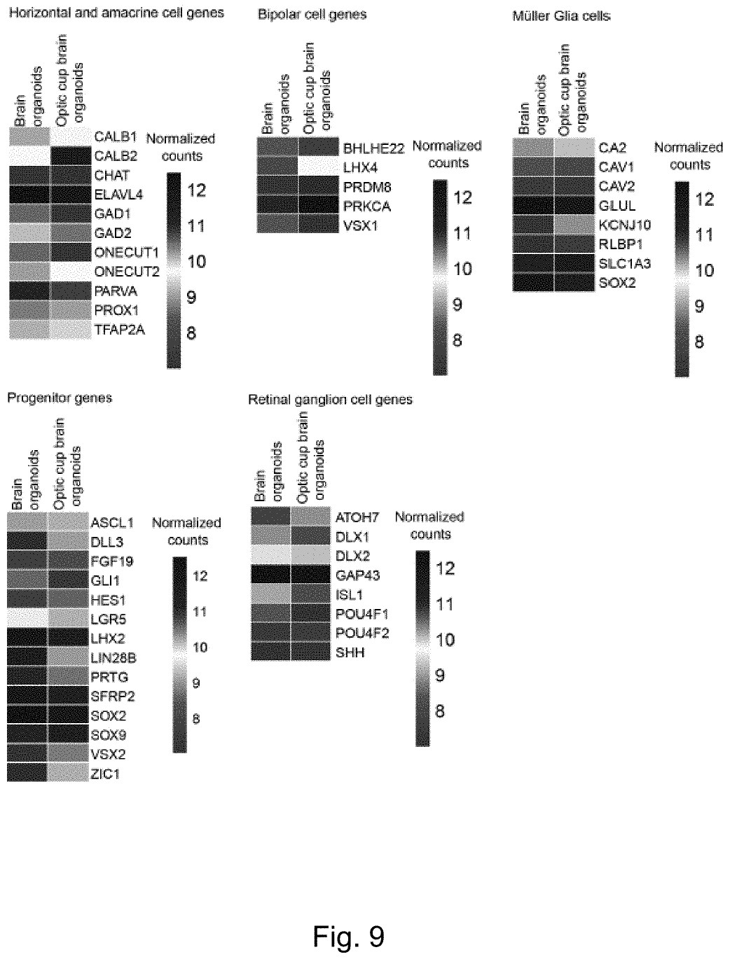

FIG. 9

Expression profiling of optic vesicle brain organoids shows developmental eye signatures. The mRNAseq analysis of brain organoids versus optic vesicle brain organoids is shown compared to human fetal retina transciptome data (STAR aligner Hg19 human reference transcriptome) Genes are organized into subgroups of retinal cells. The majority of horizontal and amacrine cell genes are higher expressed in organoids with optic vesicles compared to brain organoids. Likewise, genes typical of bipolar cells are highly up-regulated in organoids with optic vesicles, whereas genes more generally and not exclusively expressed in Müller Glia cells, progenitor cells and retinal ganglion cells are slightly up-regulated compared in optic vesicle organoids compared to early brain organoids.

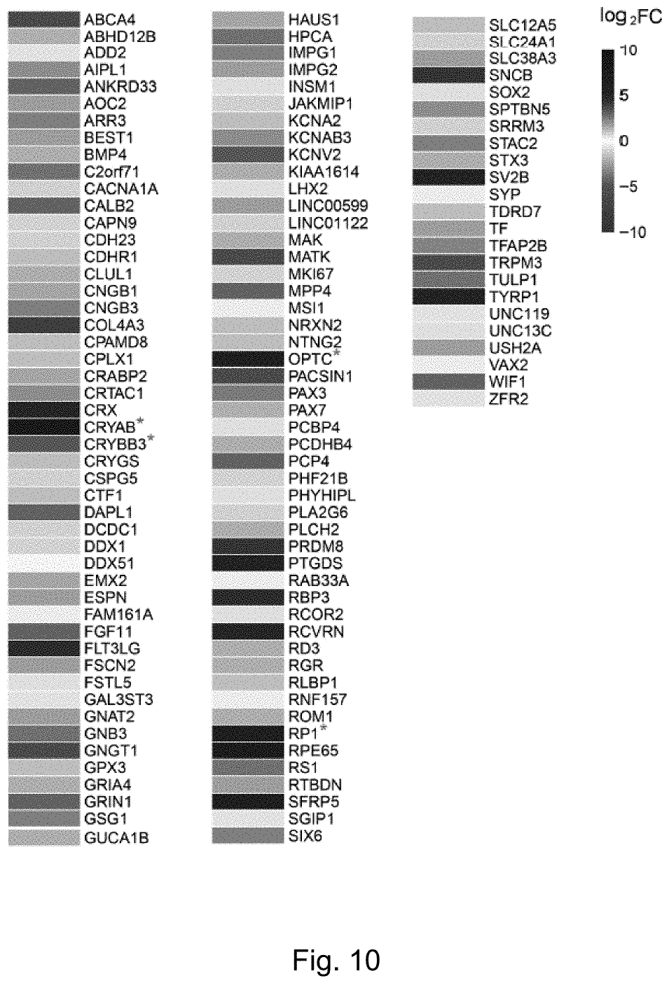

FIG. 10

Expression profiling of optic vesicle brain organoids shows optic nerve formation signatures. RNA-Seq results of brain organoids compared to optic vesicle brain organoids. Summary of highly expressed typical eye genes, such as CRX, PCP4, RCVRN (Recoverin) or RPE65 (RPE cell marker). Red asterisks mark genes expressed in lens (CRYAB, CRYBB3) and Opticin (OPTC), a gene highly expressed in the vitreous humor of the eye, the cornea, ciliary body, optic nerve, choroid, iris, and retina.

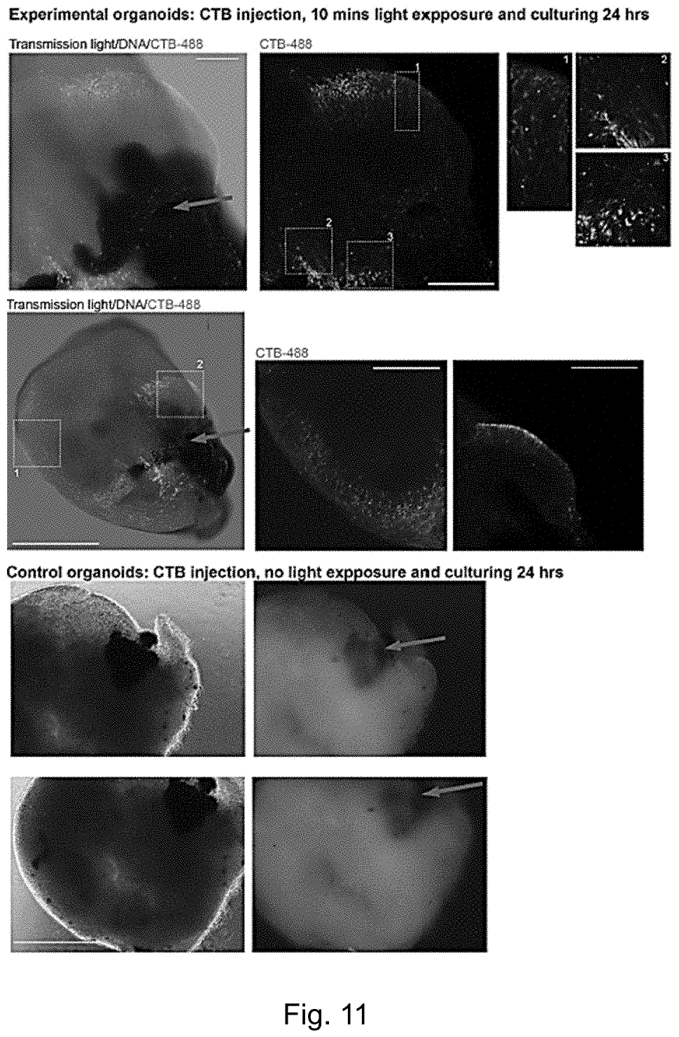

FIG. 11. Related to FIG. 4

Day 50 old optic vesicle 3D brain organoid was injected with CTB-488 directly into an optic vesicle, and exposed to light for 10 min, cultured for 24 hours and then PFA fixed. Following tissue clearing, the specimens were imaged. The image on left shows merged transmission light with optic vesicle and dark pigmented area at the injection site (arrow), nuclear staining and CTB-488. The middle panel shows the tracing of neuronal pathways by using live dye CTB-488. Right panel 1 shows magnified retinal area, the region of photosensitive neurons that firstly were active upon light stimulation and then transmitted electrical signals and the CTB-488 to next neuronal layers. Right panel 2 and 3 show magnified areas of deeper neuronal layers receiving CTB-488 and thus an electric signal from the photosensitive retinal neurons. Scale bars, 500 μm (left panel), 200 pm (middle and right panel). Bottom panel shows control organoids, which were not exposed to light. Note that there is no apparent CTB uptake or projections are seen. Representative images are given. N=12 from 4 independent batches. Representative images, cell line F14536.2.

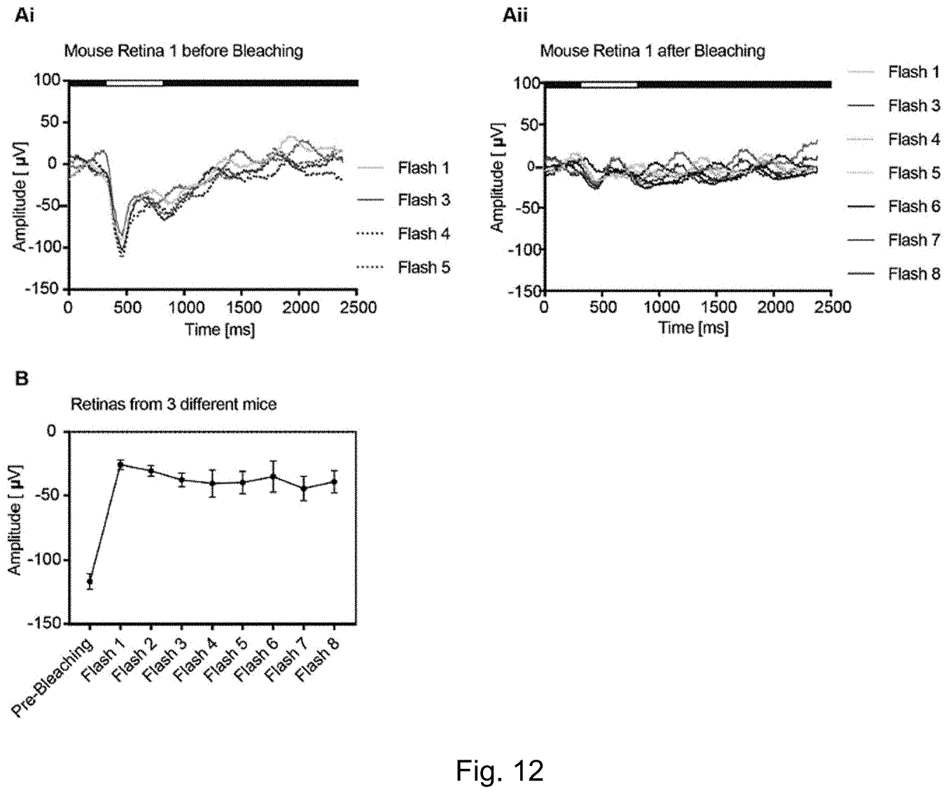

FIG. 12. Related to FIG. 5.

Photic stress experiment with isolated mouse retina. A. Mouse retina 1 light response to 200.000 mlux light flashes of 500 ms duration. The recording was done for 5 flashes with each 3 min break (i.) The same mouse retina 1 after photic stress by bright light (4600 lux for 10 min with the infrared filter between retina and bright light source) is showing no light response to 200.000 mlux light flashes even after 24 min of recovery (ii).

B. Statistical summary of photic stress experiment with retinas from three different mice. N=3 independent experiments; One-way ANOVA data followed by Tukey's multiple comparisons test. Error bars, +/−SEM.

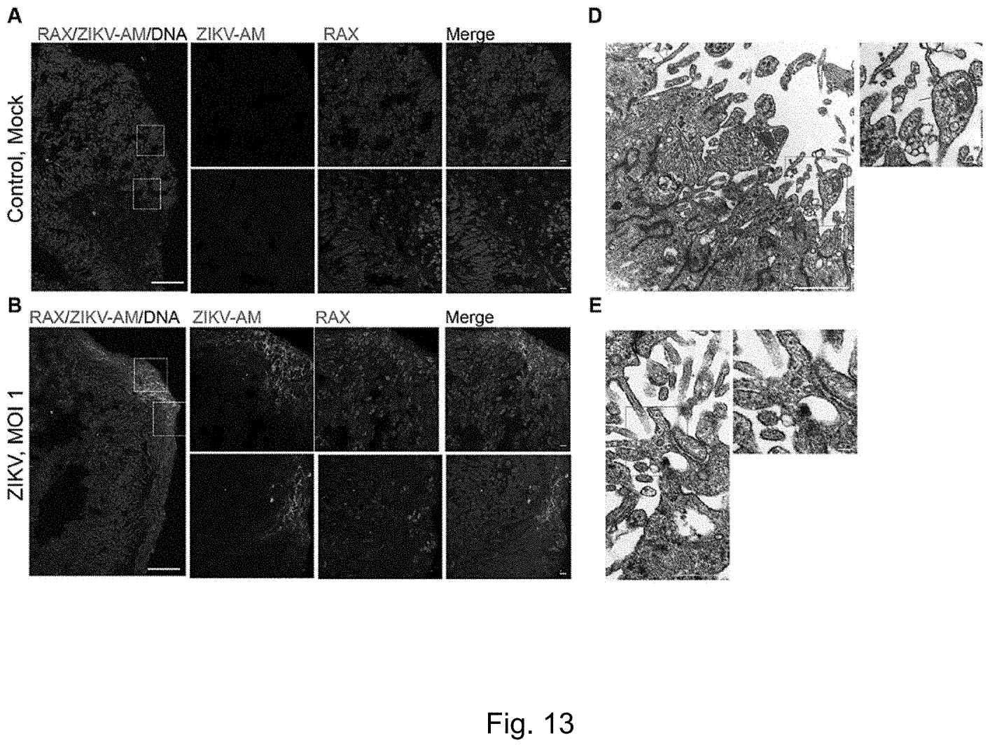

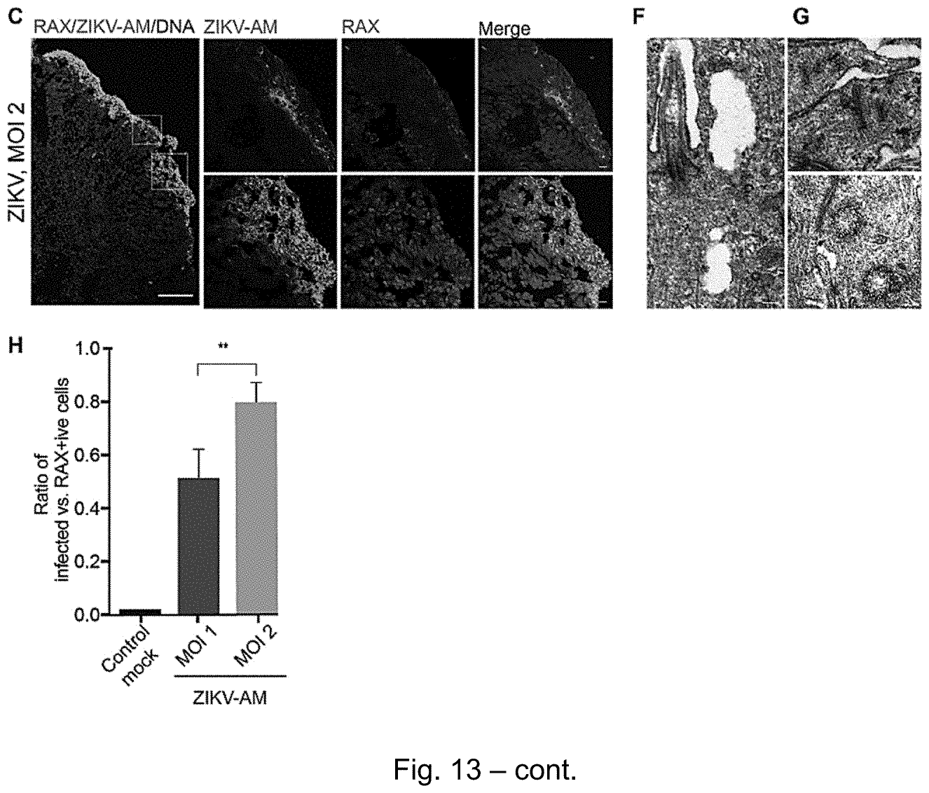

FIG. 13

A. Optic vesicle organoids to test the tropism of ZIKV to retinal progenitors. Uninfected control organoid displaying RAX-positive retinal progenitors at its periphery are negative for anti-flavi antibody against ZIKV. Scale bars, 50 μm (left panel) and 10 μm (magnified right panel). Two magnified regions are shown at right. ZIKV-AM targets RAX-positive cells after 2 days of post-infection. A noticeable increase in infectivity is found with MOI 2 (multiplicity of infection) (B-C). Representative image show only, staining for RAX. Scale bars, 100 μm (left panel), 10 μm (magnified images at right panel). Representative images, cell line IMR-90.

D-G. Electron micrographs of ZIKV-AM infected optic vesicle organoids. Overview image (D) and the magnified image at right show a viral particle (arrow). Two more representative images (E-F) show a viral particle (arrow). As previously described 22, ZIKV-AM infection induces structural damages to centrioles (G). Arrows point to broken centrioles. Scale bars, 500 nm (overview images) and 100 nm (insets). Graph quantifies an obvious increase in infectivity with MOI 2. At least 8 independent organoids under each condition of infections were analyzed. Representative images, cell line IMR-90.

H. Bar diagram showing the ratio of infected vs. RAX positive cells. Compared to MOI 1, significantly more RAX positive cells were infected with ZIKV of MOI 2 (p**<0.01). Welch's t-test, error bars show +/−SEM. The uninfected control organoids (mock) were negative for ZIKV. N=6 organoids from two independent batches.

Material & Methods

Human iPS Cell Maintenance

Human iPS cell lines IMR90, GM25256, F13535.1 and F14536.2, and were cultured in mTeSR1 medium (Stem cell technologies) on Matrigel (Corning) coated cell culture dishes at 37° C. and 5% CO2 and routinely checked for mycoplasma contamination with the MycoAlert Kit (Lonza). Cells were dissociated to small aggregates with ReLeSR (Stem cell technologies) every 5-7 days and splitted 1:5 onto fresh Matrigel coated dishes. Unless otherwise stated, most analyses shown were done with organoids derived from IMR90 and GM25256.

Neural Induction

It is important to start differentiation with a well-maintained human iPS cell culture. Cells were splitted once after thawing, mycoplasma tested and checked for appropriate stem cell morphology. Before inducing neural differentiation, iPS cells were dissociated to single cells. Therefore, iPS cells were once carefully washed with pre-warmed DMEM/F12 (Gibco) and then treated for 5 min. at 37° C. with Accutase (Sigma-Aldrich). After cell collection, centrifuging and counting, cells were diluted in neural induction medium (NIM, Stem cell technologies) plus Rockinhibitor (10 μM, Y-27632 2HCl, Selleck Chem) at a concentration of 1×105 cells per ml. 100 μl of this cell suspension was given into each well of a 96-well v-bottom low adherent plate and incubated at 37° C. and 5% CO2. Half of the medium was changed every day for 5 days until neurospheres were formed.

Neurospheres in Petri Dishes and Spinner Flasks

At day 5 of neural induction, the neurospheres were collected carefully with a cut 100 pl pipette tip and transferred to a 100 mm petri dish containing “neurosphere medium” (Table 9). Additionally, 0.1% Matrigel was added to the medium and neurospheres were incubated for 5 days at 37° C. and 5% CO2. At day 3, 2 ml of “optic vesicle 3D brain organoid medium” (Table 10) containing retinol acetate was added. At day 5, neurospheres were transferred to spinner flasks containing 100 ml “optic vesicle 3D brain organoid medium” (Table 2) to let the neurospheres developing to optic vesicle containing brain organoids. Retinol acetate was added at different concentrations ranging from 0 to 120 nM, but best results (highest yield of optic vesicle brain organoids) were obtained with a concentration of 60 nM retinol acetate. Optic vesicle organoids were collected for experiments at day 20, 30 and 50. For each cell line, we performed at least 3-4 rounds (batches) of optic vesicle 3D brain organoid generation. Each batch comprises of starting from thawing iPSCs until generating optic vesicle organoids. Morphological and functional optic vesicles brain organoids were easily identified by visibly pigmented areas and used for further analyses.

ZIKV Infection of Optic Vesicle Brain Organoids

We transferred 30 days old optic vesicle organoids from Spinner flasks to wells of a 12-wellplate and infected them with ZIKV strain ZIKV-AM (FB-GWUH-2016)1 for 2 days with MOI 1 or MOI 2. ZIKV infection was performed in Biosafety Level 2 as recommended by WHO. Any handling with ZIKV and viral-infected cultures were performed in appropriately equipped laboratories by staff trained in the relevant technical and safety procedures. German national guidelines on laboratory biosafety have been followed in all circumstances.

Immunofluorescence Microscopy

Optic vesicle brain organoids were collected at day 20, 30 or day 50 and fixed in 4% PFA (Merck Millipore) for 30 min at room temperature (RT) and transferred into new 24-well plates containing PBS-Glycine (0.225 g Glycine per 100 ml PBS). PBS-Glycine was removed and 30% Sucrose (Merck Millipore) was added to dehydrate the tissues over night at 4° C. Next day, organoids were embedded into Tissue-Tek® Cryomold® Cryomolds (Science services) containing Tissue-Tek® O.C.T.™ Compound solution (Science Services) and immediately stored at −80° C. for minimum 24 hours. Frozen specimens were embedded and cryo-sectioned at 12 μm thickness with Cryostat (Leica CM3050 S) and placed onto Poly-L-lysine (Sigma-Aldrich) pre-coated Superfrost glass slides (0.01% PLL diluted in distilled H2O and coated for 5 min at RT, subsequently dried 3 hours at RT) (Thermo Scientific). Slides with organoid sections were dried for 1 hour at RT and then kept for long-term storage at −80° C. Prior to immunofluorescent staining procedures, we thawed the slides with organoid sections for 30 min at room temperature, washed 2×3 min with PBS-Glycine (0.225 g per 100 ml) and permeabilized for 15 min in 0.5% Triton X100/0.1% Tween (Sigma Aldrich) in PBS solution at RT, and washed again 2× 3 min with PBS-Glycine. Subsequently sections were blocked with blocking solution (0.5% Fish gelatin in PBS) for 1 hour at RT. Primary antibodies were diluted in blocking solution and sections were incubated with primary antibodies for 2 hours at RT. After 3× washing with blocking solution, corresponding secondary antibodies (goat-anti mouse Alexa 488 or 647; donkey-anti rabbit Alexa 488, 594 or 647 or Donkey-anti goat Alexa 594) were diluted in blocking solution (1:1000) and incubated for 1 hour at RT protected from light (Table 12 with antibody details). Finally, sections were stained with nuclear dye Höchst 33342 (Thermofisher), washed 2× 3 min with blocking solution, and after a final wash with distilled water briefly air-dried and then mounted with Mowiol (Sigma Aldrich) and glass coverslips. Slides were dried for one hour at RT and then stored at 4° C. in dark until imaging.

Microinjection and Tissue Clearing

For optic tract experiments, we injected CTB-488 or CTB-647 (Invitrogen) (1:1 (v/v) mixed with DMEM/F12 comprising a total volume of 3 μl) into each of the optic vesicles recognized by the pigmented areas within the surrounding whitish tissue. We used a micromanipulator (Märzäuser, Wetzlar) for injection. Injected organoids were placed into wells of a 24-well plate in optic vesicle 3D brain organoid medium and then exposed to normal room light for 10 min. After exposure to light, the organoids were further cultured at 37° C. and 5% CO₂ for 24 hours before fixation with 4% paraformaldehyde (PFA) for 30 min at RT.

Tissue clearing was performed as previously described2. In brief, PFA fixed organoids were dehydrated sequentially in graded ethanol (EtOH) series of 30% EtOH (vol/vol), 50% EtOH (vol/vol), 75% EtOH (vol/vol) and 100% EtOH (vol/vol) (each step for 30 min at 4° C. in a gently shaking tube 1 ml tube) and subsequent tissue clearing was performed with Ethyl cinnamate (ECI; Sigma Aldrich) for approximately 30 min at room temperature. Tissue cleared organoids were then placed into cover slip bottom p-slides (Ibidi) and stored at 4° C. until 3D imaging.

3D Imaging and Processing

Images were acquired with confocal microscope (Leica SP8) and converted into .tiff files with Fiji software (ImageJ 1.52i). Images were then processed with Photoshop (Adobe CS5) and copied as .psd files into Adobe Illustrator. Tissue cleared organoids were placed into μ-Slide angiogenesis (Ibidi, Germany) and imaged using Zeiss LSM 880 confocal microscope with EC Plan-Neoflaur 10×/0.3 and Plan-Apochromat 20×/0.8 M27 objectives. The raw files were exported to Imaris x64 version 7.7.1 to perform surface and volume rendering. The files were further processed using Image J, Adobe photoshop CC 2017 and Adobe illustrator CC 2017.

Electrophysiology: ERG with Organoids

Optic vesicle organoids of day 40 to 50 were transferred to wells of a 12-wellplate each with 2 ml of optic organoid medium and kept in absolute darkness at 37° C. and 5% CO₂ for 24 hours before electroretinogram (ERG) experiments. For ERG, each organoid was placed under red light in an electrode chamber. The electrode chamber was then placed into the dark chamber of the ERG apparatus and connected to the superfusion system containing optic organoid medium. For dose dependent light sensitivity, organoids were exposed to 500 milliseconds long flashes of 2000, 20.000 and 200.000 mlux. Each light intensity was repeated three times in a time interval of 3 min. before the next higher light intensity was applied and responses were recorded. Speed of superfusion was 0.5 ml/min. In controls, we used tissues without optic vesicles, which seldom displayed a basal level of response.

For photic stress experiment, optic vesicle brain organoids were superfused with 0.5 ml/min optic organoid medium and exposed to 500 ms long flashes of 200.000 mlux every 3 min. for adjustment to the system for 15 min. (pre-bright light). Then organoids were desensitized in electrode chamber by bright light exposure with light intensity of 4600 Lux for 10 min. As bright light source served a 150 W white light bulb. Between bulb and electrode chamber was an infrared filter placed to subtract the thermal impact of the bright light source. After 10 min. of bright light exposure, organoids were allowed to regenerate in dark chamber for 30 s before light flashing (500 ms, 200.000 mlux) and recording under above mentioned conditions continued. As negative control, when organoids were regenerated from photic stress usually after 30 min. of flashing and recording, same organoids were treated under same conditions again except bright light exposure was replaced by 0 lux.

For a third experiment, organoids were superfused in optic organoid medium with higher speed of 2 ml/min. After 36 min of equilibration time, organoids got adjusted to the higher superfusion speed and responded with a stable electrical signal of −200 mV as response to flashes of 200.000 mlux and 500 ms. For isolating the A-wave by blocking signal transduction from photoreceptors to next the retinal cell layers, 10 mM aspartate was added to the medium after the equilibration time and electric response was continuously recorded. Finally, aspartate was washed out by adding optic organoid medium without aspartate into the system while recording of electric signals as response to flashes.

Electrophysiology: ERG with Isolated Mouse Retina

Mice were dark-adapted overnight, sacrificed by cervical dislocation under a dim red light and the eyes were extirpated immediately. Enucleated eyes were protected from light and transferred into carbogen-saturated (95% O2/5% CO2) Ames medium and into the recording chamber as described previously3. The b-wave amplitude was measured from the trough of the awave to the peak of the b-wave. For each experiment, a new murine retina was transferred to the recording chamber. The institutional and governmental committees on animal care approved the animal experimentation described in the text (Landesamt fur Natur, Umwelt and Verbraucherschutz (LANUV) Nordrhein—Westfalen, Recklinghausen, Germany; 84-02.04.2016.A4555) which was conducted following accepted standards of humane animal care.

Electrophysiology: Whole Cell Patch Clamp

Living organoids were embedded in 2% low-melt agarose (Invitrogen) and attached to a cutting plate beside a 4% agarose block. Vibratome sectioning was done at room temperature. For patch clamp experiment slices were cut into 350-400 μm sections. Cells with spontaneous activity were identified before patch clamping by staining with fluo8AM (Abcam) (5 μg/ml) for 15 min at room temperature. Overview of spontaneous activity was recorded with a LSM setup (Zeiss 780). Each cell was recorded for 5 to 10 min. in “on cell”/“cell” attached configuration while the fluorescent signal was recorded as well. Then the cell membrane was broken to perform “whole-cell” configuration for channel currents recording. During whole-cell recording, the cell is clamped at a resting potential of −70 mV. Depolarization steps were applied in a 10-mV interval for the current-voltage curves. The recording temperature was approx. 37° C. in ACSF (artificial spinal fluid).

Transmission Electron Microscopy (TEM)

Organoids of day 50 were washed once in PBS and then fixed in 2.5% Glutaraldehyde and 1.8% Sucrose in PBS over night at 4° C. Samples were rinsed in PBS, post-fixed with 1% osmium tetroxide for 1 hr at 4° C. After fixation, the samples were further dehydrated in a graded ethanol series, and embedded in Epon-Araldite resin for 48 hrs at 60° C. Reichert Ultracut E ultramicrotome was used to obtain the Ultrathin sections (40-60 nm), further stained with uranyl acetate and lead citrate, and observed with a FEI Tecnai G2 Spirit 255 transmission electron microscope operating at 100 kV.

Scanning Electron Microscopy (SEM)

Organoids of day 50 were washed once in PBS and then fixed in 2.5% Glutaraldehyde and 1.8% Sucrose in PBS over night at 4° C. Samples were then rinsed with PBS and post-fixed with 1% osmium tetroxide in PBS for I hr at 4° C. After alcohol dehydration, samples were critical point dried in a Balzers CDP 010. The material was then mounted on aluminium stubs, coated with gold using a Balzers Med 010 and examined with a Philips XL20 scanning electron microscope (SEM) operating at 15 KV.

RNA Isolation, RNA-Sequencing and Analysis

Organoids (from IMR-90) were washed 1× in cold PBS and then immediately transferred to a fresh RNAse free vessel containing 300 μl Tri-Reagent (Sigma Aldrich, USA) by free-thawing, and total RNA was isolated and DNase-treated using the DirectZol RNA kit (Zymo Research). Approximately 2 μg of total RNA was used to subselect poly(A)+ transcripts, and generate strand-specific cDNA libraries (TrueSeq kit; Illumina). These libraries were then sequenced to ˜50 million read pairs each (75-bp reads) on a HiSeq4000 platform (Illumina). After assessing the sequencing data quality with FastQC, paired-end sequencing reads were mapped with STAR aligner4 to Hg19 human reference assembly and quantified with featureCounts5. Raw read counts were normalized with RUVSeq6 package in R in order to remove unwanted variation based on replicate samples (RUVs). Data was subsequently analyzed for changes in differential gene expression (DGE) with DESeq27 package. List of 3923 significantly DGE genes (log2FC ±1.5 and padj<=0.05 cutoff) was subjected to affinity propagation clustering based on similarity matrix, with apcluster8 package. From resulting dendrogram containing 75 clusters, cutoff of k=9 resulted in 9 super clusters which were used for GO term search analysis with clusterProfiler9 Significant DGE genes classified in supercluster 9 were subjected to gene set enrichment analysis with GSEA (ref 8**; v3.0) using C5 collection of GO gene sets from Molecular Signatures Database10.

RPE Isolation and Culturing

For isolation of RPEs, optic vesicle organoids were grown until day 50-60 and then dissociated with Trypsin (0.05%, 20 min, 37° C.) to small cell aggregates. Trypsin was inhibited by 20%-DMEMFBS and dark aggregates were collected in a separate 15 ml Falcon, and centrifuged at 1000× g. The pellet was resuspended in RPE Medium (Table 11) and cells were seeded onto laminin-coated cell culture treated dishes. Cells were cultured approx. for two weeks until large colonies of pigmented cells were grown as monolayer sheets.

Statistics

Statistical analyses were performed using Graphpad Prim 7 for Mac OS X (Version 7.0e, Sep. 5, 2018).

TABLE 9

| “Neurosphere medium”: | | |
|---|---|---|
| Component: | Final conc. | |
| DMEM/F12 | 48.4% (v/v) | |
| Neural basal medium | 48.4% (v/v) | |
| N2 | 0.4× | |
| B27 w/o Vitamin A | 0.2× | |
| Glutamax | 1× | |
| MEM | 0.5× | |
| Insulin | 0.2755 | μM |
| SB431542 | 2.5 | μM |
| Pen/Strep | 100 | U/ml |
| β-Mercaptoethanol | 5 | μM |

TABLE 10

| “Optic vesicle 3D brain | | |
|---|---|---|
| organoid medium”: | | |
| Component: | Final conc. | |
| DMEM/F12 | 48.4% (v/v) | |
| Neural basal medium | 48.4% (v/v) | |
| N2 | 0.4× | |
| B27 + RA | 0.2× incl. 60 nMol | |
| | retinol acetal | |
| Glutamax | 1× | |
| MEM | 0.5× | |
| Dorsomorphin | 0.5 | μM |
| Insulin | 0.2755 | μM |
| SB431542 | 5 | μM |
| Pen/Strep | 100 | U/ml |
| β-Mercaptoethanol | 5 | μM |

TABLE 11

| “RPE medium”: | |
|---|---|
| Component: | Final conc. |
| DMEM/F12 | 87% (v/v) |
| FBS | 10% (v/v) |
| Glutamax | 1× |
| MEM | 1× |
| Pen/Strep | 100 U/ml and 100 μg/ml |
| β-Mercaptoethanol | 5 μM |

TABLE 12

| Primary antibodies: | Source: | Cat. No. | raised against: | Dilution: |
|---|---|---|---|---|
| Anti-flavivirus group | Millipore | D1-4G2-4-15 | mouse | 1:2000 |
| antigen | | | | |
| Alpha A/alpha B | Enzo Life | ADI-SPA-224-D | rabbit | 1:50 |
| Crystallin | Sciences | | | |
| Anti-CRX | R&D systems | #O43186 | sheep | 1:100 |
| Anti-Ctip2 (25B6) | abcam | ab18465 | rat | 1:500 |
| Anti-Doublecortin | Synaptic | 326003 | rabbit | 1:400 |
| (DCX) | Systems | | | |
| Anti-FoxG1 | abcam | ab18259 | rabbit | 1:400 |
| Anti-Keratin K3/K76 | Millipore | #CBL218-I | rabbit | 1:1000 |
| (clone AE5) | | | | |
| Anti-Opsin, Red/Green | Millipore | #AB5405 | rabbit | 1:200 |
| (L/M) | | | | |
| Arl13B | Proteintech | 17711-1-AP | rabbit | 1:200 |
| Human NRL antibody | R&D systems | AF2945 | goat | 1:200 |
| Myelin Basic Protein | Cell Signaling | #78896 | rabbit | 1:50 |
| (D8X4Q) | | | | |
| Nestin (4D11) | Novus | NBP1-92717 | mouse | 1:400 |
| | biologicals | | | |
| PAX6 | DSHB | PAX6 | mouse | 1:50 |
| PCP4 | Proteintech | 14705-1-AP | rabbit | 1:200 |
| Phalloidin-TRITC | Sigma Aldrich | P1951 | — | 0.1 μg/ml |
| PHF-1 | kind gift from | Tau phos | mouse | 1:200 |
| | Peter Davis | Ser396/Ser404 | | |
| | | (PHF-1) | | |
| Rax | abcam | ab23340 | rabbit | 1:50 |
| Rhodopsin | Novus | NBP2- | mouse | 1:1000 |
| | Biologicals | 25159SS | | |
| Synapsin-1 | Cell Signaling | #5297 | rabbit | 1:200 |
| Secondary antibodies: | Source: | Cat. No. | | Dilution: |
| Alexa Fluor 488 - | Life | A21206 | donkey | 1:800 |
| Donkey anti-rabbit IgG | technologies | | antirabbit | |
| (H + L) | | | | |
| Alexa Fluor 488 - goat | Life | A28175 | goat antimouse | 1:800 |
| anti-mouse IgG (H + L) | technologies | | | |
| Alexa Fluor 594 - | Life | A21207 | donkey | 1:800 |
| Donkey anti-rabbit IgG | technologies | | antirabbit | |
| (H + L) | | | | |
| Alexa Fluor 594 - goat | Life | A11032 | goat antimouse | 1:800 |
| anti-mouse IgG (H + L) | technologies | | | |
| Alexa Fluor 647 - | Life | A31573 | donkey | 1:800 |
| Donkey anti-rabbit IgG | technologies | | antirabbit | |
| (H + L) | | | | |
| Alexa Fluor 647 - goat | Life | A21236 | goat antimouse | 1:800 |
| anti-mouse IgG (H + L) | technologies | | | |
| Alexa Fluor 647 - | Life | A-21448 | donkey | 1:800 |
| donkey anti-Sheep | technologies | | antisheep | |
| IgG (H + L) | | | | |
| Alexa-Fluor 594 - | Life | A-21209 | donkey antirat | 1:800 |
| donkey anti-Rat IgG | technologies | | | |
| (H + L) | | | | |
| Alexa-Fluor 594 - goat | Life | A-11032 | goat antimouse | 1:800 |
| anti-Mouse IgG (H + L) | technologies | | | |

Generation of 3D Brain Organoids with Optic Vesicles

A protocol of inducing differentiation into neural epithelium directly from iPSCs has been described previously and is incorporated herein by reference (Albanna, W. et al. Electroretinographic Assessment of Inner Retinal Signaling in the Isolated and Superfused Murine Retina. Current eye research 42, 1518-1526 (2017); Gabriel, E. & Gopalakrishnan, J. Generation of iPSC-derived Human Brain Organoids to Model Early Neurodevelopmental Disorders. Journal of visualized experiments: JoVE, (2017); Gabriel, E. et al. Recent Zika Virus Isolates Induce Premature Differentiation of Neural Progenitors in Human Brain Organoids. Cell stem cell, (2017)). Through this protocol, the neural differentiation proceeded in a controlled manner limiting the formation of mesoderm and endoderm, which are not required for ectodermal differentiation (Streit, A., Berliner, A. J., Papanayotou, C., Sirulnik, A. & Stern, C. D. Initiation of neural induction by FGF signalling before gastrulation. Nature 406, 74-78, (2000)). These state-of-the-art brain organoids expressed several retina and eye-related genes (data not shown), but never developed into visible optic vesicles.

This has now surprisingly been achieved by the present invention. This is demonstrated by the present example, starting with a low-density cell number (1×10⁴ iPSCs as starting number for one organoid) and adding retinol acetate ranging from 0 to 120 nM to the culture medium at an earlier time point when the neuroectoderm expands (for detailed information see the Materials and Methods section). An addition of 60 nM of retinol acetate reproducibly induced the formation of pigmented structures, possibly primordial eye fields at around day 30. Pigmented areas restricted to one pole of the organoid suggested the presence of the forebrain-like region where the primordial eye field develops (FIG. 1, panel B).

Next, the inventors tested for the presence of eye field patterning markers in the organoids. Indeed, immunostaining for identity markers revealed the presence of RAX-, NRL-, Pax6- and FoxG1-positive progenitor cells in this region (FIG. 1, panels C and D). The primordial eye field, which arises in a region lateral to the diencephalon, is enriched with RAX, a transcription factor essential for eye field patterning and to define optic vesicles developing from the diencephalon (Furukawa, T., Kozak, C. A. & Cepko, C. L. rax, a novel paired-type homeobox gene, shows expression in the anterior neural fold and developing retina. Proceedings of the National Academy of Sciences of the United States of America 94, 3088-3093 (1997); Mathers, P. H., Grinberg, A., Mahon, K. A. & Jamrich, M. The Rx homeobox gene is essential for vertebrate eye development. Nature 387, 603-607 (1997); Stigloher, C. et al. Segregation of telencephalic and eye-field identities inside the zebrafish forebrain territory is controlled by Rx3. Development 133, 2925-2935, (2006)).

Likewise, the transcription factor NRL specifies cell fate of rod photoreceptors (Kim, J. W. et al. NRL-Regulated Transcriptome Dynamics of Developing Rod Photoreceptors. Cell reports 17, 2460-2473, (2016)). FoxG1 is initially expressed in the prosencephalic neuroepithelium and later involves in the telencephalon and eye field segregation (Stigloher, C. et al. Segregation of telencephalic and eye-field identities inside the zebrafish forebrain territory is controlled by Rx3. Development 133, 2925-2935, (2006)).

Culturing these organoids for up to 50 days resulted in the progressive development of these pigmented regions forming one or two strongly pigmented optic vesicle-like structures (hereafter optic vesicle brain organoids). 86 out of 95 organoids from the IMR-90 iPSC line could develop easily recognizable bilateral symmetric optic vesicles. This method is robust and reproducible because, for example, 314 brain organoids could be generated from 16 independent batches across four iPSC donors with an overall success rate of 226 (72%) brain organoids assembling optic vesicles. None of the organoids derived from any of the four tested iPSC donors formed more than two pigmented regions. Intriguingly, these pigmented optic vesicles were exclusively restricted to one pole of the organoids near to each other, suggesting an area that is topographically patterned at the forebrain region (FIG. 1, panels E-G).

Optic Vesicles of 3D Brain Organoids Display Immature Stratified Neural Retina

To test the presence of stratified neural retina, sections transversely cut through an optic vesicle were immunostained. The presence of densely packed elongated nuclei from basal to the apical surface facing the outer RPE was ascertained. This arrangement is not identical but somewhat similar to the inner and outer nuclear cell layers as observed in developing mouse or human feta retina (Hoshino, A. et al. Molecular Anatomy of the Developing Human Retina. Developmental cell 43, 763-779 (2017)). Unlike in vivo counterpart, optic vesicle brain organoids do not show distinct layers suggesting that the organoids are not yet mature enough even though different cell types exist but not segregated (FIG. 2, panel A and FIG. 6, panel A).

The pigmented RPE layer has strongly interfered with the immunostaining and the visualization of nuclei. This interference could be due to the inherent property of organoids failing to mirror an exact anatomical cytoarchitecture of in vivo counterparts. However, in sections where RPE positioning did not hinder the visualization, rhodopsin and L/M-opsin expression was observed at the RPE layers, which specify rods and cones, respectively. Importantly, this region was strongly positive for cone-rod homeobox protein (CRX), a photoreceptor determinant (FIG. 2, panel A) (Furukawa, T., Morrow, E. M. & Cepko, C. L. Crx, a novel otx-like homeobox gene, shows photoreceptor-specific expression and regulates photoreceptor differentiation. Cell 91, 531-541 (1997)). and PCP4 specifying bipolar and amacrine cells (Chintalapudi, S. R. et al. Isolation and Molecular Profiling of Primary Mouse Retinal Ganglion Cells: Comparison of Phenotypes from Healthy and Glaucomatous Retinas. Frontiers in aging neuroscience 8, 93, (2016)). (FIG. 4, panel F). To image the architecture of RPE, the inventors stained for F-actin and Arl13B, which specify the outer cell membrane and primary cilia. The inventors noticed highly organized pigmented cells displaying the typical ‘honeycomb-like’ morphology, each containing a primary cilium (FIG. 2, panel C). Tissue clearing and whole-mount imaging followed by 3D reconstruction revealed the organization of RPE in an optic vesicle that is spatially restricted to one pole of the organoid (FIG. 6, panel B). Finally, scanning electron microscopy (EM) of transverse sections identified that RPE presents as a tightly packed monolayer with each cell harboring a primary cilium in its surface, an arrangement that is reminiscent of the in vivo counterpart (FIG. 2, panel D). Disassociation of the pigmented region and subsequent plating also resulted in a monolayer sheet of RPE cells displaying ‘honeycomb-like’ morphology (FIG. 6, panel C). Basal to RPE, the inventors noticed serially organized cilia surrounded by a rhodopsin-positive area suggesting that these Arl13B-positive structures are possibly connecting cilia of photoreceptor cells (FIG. 6, panel D).

Visualizing these structures using serial sectioning transmission EM revealed distinctive features of RPE cells with numerous cytoplasmic melanosomes (Strauss, O. The retinal pigment epithelium in visual function. Physiological reviews 85, 845-881 (2005)). Each RPE cell harbored a primary cilium in its surface (FIG. 2, panel E). The inner layer of RPE contained rod cells, which displayed a typical bulgy outer segment enclosed by an RPE cell at the apical side and connected to an inner segment via a connecting cilium (FIG. 2, panel F and FIG. 7). Importantly, the inventors also identified ectosomes release from the outer segment, a cellular process that occurs explicitly during outer segment formation (Salinas, R. Y. et al. Photoreceptor discs form through peripherin-dependent suppression of ciliary ectosome release. The Journal of cell biology 216, 1489-1499 (2017)). (FIG. 2, panel F(ii)). The inventors noticed another kind of a cell type that was morphologically distinct from rods. These cells contained a triangular-shaped outer segment containing many vesicles and flattened cisternae suggesting that they are cone cells (FIG. 2, panel G). In summary, optic vesicle brain organoids contain diverse retinal cell types organized as an immature stratified neural retina.

Transcriptome of Developing Optic Vesicle Brain Organoids Reveal Diverse Retinal Cell Types and Functionality

To delineate differential gene expression and cellular diversity, the inventors generated and compared transcriptome profiles of brain organoids to those of optic vesicle brain organoids using bulk mRNA sequencing. Following data processing, read mapping and variance normalization, principal component analysis (PCA) showed two well-defined subgroups contributing up to ˜80% of observed sample distance corresponding to optic vesicle brain organoids and brain organoids. Differential gene expression analysis revealed ˜4,000 genes with significantly changing mRNA levels in optic vesicle brain organoids compared to brain organoids (q-value <0.05, FIG. 8, panels A and B). Of these, ˜¾ were up regulated and most probably signifying the activation of the developmental pathway leading to optic vesicle formation. This interpretation was substantiated by affinity propagation clustering to identify gene signatures from differentially-expressed genes resulting in 75 sub-clusters further grouped into 9 super-clusters displaying distinct molecular pathways (FIG. 8, panel C). In addition to synapse maturation, cell proliferation, and cell cycle regulation, which signify neural tissue morphogenesis and differentiation, up-regulation of genes involved in the detection of light stimuli, in visual perception, as well as in compound eye morphogenesis was observed (super-clusters 4 and 7, respectively; FIG. 8, panel C).

Gene set enrichment analysis (GSEA) of super-cluster 4 that carries the visual perception-related signature coupled to leading-edge analysis, revealed an array of genes associated with early retina development, including early retinal progenitor and retinal ganglion markers, as well as transcription factors of photoreceptor cells (e.g., RPE65, SFRP2, FGF19, CRX, RCVRN, RAX, VSX2, LIN28B, PRTG, SFRP2, ATOH7, DLX2 or POU4F2; FIG. 8, panel D). Furthermore, direct comparison to transcriptomic data from fetal retina showed that there indeed exists an apparent correlation between the fetal retina and optic vesicle brain organoids in terms of expressed genes relevant to horizontal, amacrine, bipolar, Müller glia, progenitor and retinal ganglion cells (FIG. 9). Trying to find out where the optic vesicle brain organoids fit in the time scale of embryonic development, RNA-seq data were compared with the collection of gene expression data provided by the LifeMap Embryonic Development & Stem Cell Compendium (Edgar, R. et al. LifeMap Discovery: the embryonic development, stem cells, and regenerative medicine research portal. PloS one 8, e66629, (2013)). This comparison revealed that optic vesicle brain organoids are enriched for genes relevant to compound eye development, including cell types derived from surface ectoderm giving rise to the lens and cornea (e.g., CRYAB, CRYBB3, and OPTC; FIG. 10).

In summary, most aspects of the transcriptomic data are in strong correlation with the development of a stratified neural retina in the optic vesicles of brain organoids, while also exhibiting signatures of synapse maturation. Indeed, organoids strongly expressed Synapsin1-positive region that is restricted to cortical plates and is also specified by mature neuronal markers like CTIP, myelin basic protein (MBP) and laminin (FIG. 3, panels A-D). Consistently, the GSEA data revealed a downregulation of genes regulating cell proliferation, indicating the quantitative commitment of these organoids to maturation. Corroborating to these findings, optic vesicle organoids exhibited an enrichment of cells expressing genes for sensory perception of light stimulus, strengthening the presence of mature functional cell types (FIG. 3, panels E-J). To substantiate the presence of mature neurons, whole-cell patch-clamp recordings were performed. In on-cell configuration, spontaneous action potentials could be measured, which were sometimes sensitive (FIG. 3, panel K) and sometimes resistant to Tetrodotoxin (TTX). TTX is a neurotoxin that selectively blocks sodium channels (Makarova, M., Rycek, L., Hajicek, J., Baidilov, D. & Hudlicky, T. Tetrodotoxin: History, Biology, and Synthesis. Angewandte Chemie, (2019)). The resistance to TTX treatment is typical for retinal cells, which possess TTX-resistant voltage-gated sodium channels (O'Brien, B. J. et al. Tetrodotoxin-resistant voltage-gated sodium channels Na(v)1.8 and Na(v)1.9 are expressed in the retina. The Journal of comparative neurology 508, 940-951, (2008)).

Optic Vesicle Brain Organoids Contain Lens and Corneal Epithelium

The inventors then tested whether optic vesicles exhibit the presence of lens and corneal epithelium, which are derived from surface ectoderm but not from neuroectoderm. Organoids showed two defined bilateral structures strongly positive for αA/αB-Crystallin positioned within each of the optic vesicle (FIG. 4, panel A). Importantly, these lens structures are enclosed within a single layer of F-actin and keratin-3-positive columnar epithelial cells suggesting that corneal epithelium neighbors the lens-like structures (FIG. 4, panel B). Ultrastructurally, the lens displayed as a distinctly rounded structure within a space, possibly the anterior chamber enclosed by the corneal epithelium (FIG. 4, panels C and D). Lens structures are further characterized by a substantial reduction of cytoplasmic organelles, a process that occurs during the differentiation of lens fiber cells (McAvoy, J. W., Chamberlain, C. G., de longh, R. U., Hales, A. M. & Lovicu, F. J. Lens development. Eye 13 (Pt 3b), 425-437, (1999)). Indeed, the lens cells contain cytoplasmic aggregates of fibrillar material (FIG. 4, panel D). Together, with the application of low seeding cell density in a defined but undirected differentiation condition, brain organoids with bilaterally symmetric optic vesicles were engineered. Although the described in vitro-tissues do not fully mimic in vivo-like architecture, they display diverse cell types of complex eye development (FIG. 4, panel E).

Optic Vesicle Brain Organoids Contain Primitive Image Forming Nuclei and Exhibit Retinal Connectivity to Brain Regions

In the mammalian brain, axons of retinal ganglion cells reach out to connect with their brain targets, an aspect that has never been shown in an in vitro system. First, the presence of the lateral geniculate nucleus (LGN) was assessed. The LGN is the main image forming nucleus of the visual thalamus that develops when eye-specific axonal segregation is completed (Sretavan, D. W. & Shatz, C. J. Prenatal development of retinal ganglion cell axons: segregation into eye-specific layers within the cat's lateral geniculate nucleus. The Journal of neuroscience: the official journal of the Society for Neuroscience 6, 234-251 (1986)). Immunoreactivity for PCP4, a developmental marker of LGN, amacrine, and bipolar cells, was identified at the vicinities that surround the optic vesicle suggesting that PCP4 could also specify ganglionic cells 30 (FIG. 4, panel F(i)). PCP4-positive cells showed a typical morphology of cell soma with an axon extended up to 100 μm (FIG. 4, panel F). Importantly, the PCP4-positive structures penetrated way out of the optic vesicle migrating from anterior to posterior of the organoid (FIG. 4, panels F(iii-iv)). The distinct PCP4 clusters posterior to optic vesicles suggest that PCP4-positive neurons could have migrated from the retina to form higher-order visual regions that are yet to be characterized. Intriguingly, staining for Tau, an axonal marker also co-localized with the extending PCP-4 fibers migrating towards inner region of organoids. Thus, the regions demarcated by PCP4- and Tau- is, to some extent, morphologically similar to the primitive optic disc and optic stalk structures (FIG. 4, panel F(ii)).

To further test that optic vesicles contain projection tracts into organoid tissues, the inventors microinjected AlexaFluor488-cholera toxin b-subunit (CTB) directly into one optic vesicle and AlexaFluor647-CTB into the other optic vesicle. CTB labels retinal nerve fiber of axon bundles Huberman, A. D., Dehay, C., Berland, M., Chalupa, L. M. & Kennedy, H. Early and rapid targeting of eye-specific axonal projections to the dorsal lateral geniculate nucleus in the fetal macaque. The Journal of neuroscience: the official journal of the Society for Neuroscience 25, 4014-4023, (2005); Mikkelsen, J. D. Visualization of efferent retinal projections by immunohistochemical identification of cholera toxin subunit B. Brain research bulletin 28, 619-623 (1992); Yao, F. et al. Did you choose appropriate tracer for retrograde tracing of retinal ganglion cells? The differences between cholera toxin subunit B and Fluorogold. PloS one 13, e0205133, (2018)). After 24 hrs of light exposure (see methods for details), a strong uptake of CTB into retinal region with numerous axon-like projections was noticed. Control organoids injected with CTB but not exposed light did not exhibit CTB uptake (FIG. 4, panel G and FIG. 11). 3D reconstruction of confocal slices revealed that the fiber-like optic tracts merge although they appear to emerge from two different optic vesicles (not shown). These observations raise the possibility that the optic vesicles are connected to inner regions of brain organoids via axonal-like projections.

Optic Vesicle Brain Organoids are Light Sensitive and Can Recover Their Light Sensitivity After Photobleach Blinding

To test the functionality of optic vesicle brain organoids, electroretinography (ERG) recordings were performed. These recordings allow the quantification of retinal signaling. In the absence of light, vertebrate photoreceptors are slightly depolarized, and light absorption leads to membrane hyperpolarization (Schiller, P. H., Sandell, J. H. & Maunsell, J. H. Functions of the ON and OFF channels of the visual system. Nature 322, 824-825, (1986)). Typically, the first graded electric response represents the hyperpolarization of the photoreceptors and is quantified as the a-wave response. Trans-synaptic excitation spreading mainly leads to the depolarization of ‘ON’-bipolar cells, which can be modulated by the neuronal network between photoreceptors and ganglion cells. The resulting net depolarization is recorded as the ERG b-wave.

The response to every single 500ms white light flash was recorded at an interval of 3 min (see method section for further details). To quantify neuronal signaling, the amplitudes and their implicit time was calculated. Notably, a dose-dependent light exposure already revealed increasing negative amplitudes of the ERG corresponding to increasing light intensities (FIG. 5, panels A(i) and A(ii)). The first question that arose in this experiment was: Does the negative deflection represent the activity of photoreceptors solely, or are there apparent negative waves containing a positive b-wave deflection? Indeed, in the vertebrate retina, the positive b-wave responses superimpose the negative a-wave response (Albanna, W. et al. Longer lasting electroretinographic recordings from the isolated and superfused murine retina. Graefe's archive for clinical and experimental ophthalmology =Albrecht von Graefes Archiv fur klinische and experimentelle Ophthalmologie 247, 1339-1352, (2009); Cameron, M. A. & Lucas, R. J. Influence of the rod photoresponse on light adaptation and circadian rhythmicity in the cone ERG. Molecular vision 15, 2209-2216 (2009); Yamaguchi, S., lijima, H. & Hosaka, O. [The effect of stimulus intensity and background luminance on the b-wave implicit time of photopic electroretinogram in normal human eyes]. Nippon Ganka Gakkai zasshi 96, 978-984 (1992)].

Therefore, to measure the response from photoreceptors, trans-synaptic signaling using a glutamate receptor antagonist was inhibited to detect the full-length amplitude of the a-wave. ERGs in the presence of 10mM aspartate blocked the b-wave, which eventually increased the negative amplitude of the fully developing a-wave to its maximum value. However, during the consecutive washout of aspartate, the re-occurrence of positive b-wave was observed, indicating the presence of trans-synaptic signal transduction in organoids (FIG. 5, panel B). This finding demonstrates that the changes in the electric potential represent mainly the responses of photoreceptors but only when aspartate is present. Removing aspartate revealed the positively deflecting b-wave, indicating that trans-synaptic signalling is present in the organoids.

Photobleaching by an intense flashlight stimulation can inactivate physiologically active photoreceptors, which can spontaneously be reversed to their initial responses by dark adaptation (Ernst, W. & Kemp, C. M. Reversal of photoreceptor bleaching and adaptation by microsecond flashes. Vision research 19, 363-365 (1979)). To investigate whether organoids reflected this physiological phenomenon, the inventors photic-stressed them by exposure to an increased light intensity of 4600 lux for 10 minutes (“Prebright light”). The amplitude of the electrical responses was transiently reduced after light stressing when recorded with short, low-intensity recording light pulses. Interestingly, the organoids could recover their photosensitivity as normalized electric responses were detected during consecutive dark adaptation (FIG. 5, panel C). These experiments reveal that organoids contain physiologically active photoreceptors that are capable of recovering light sensitivity after photobleaching. Importantly, when repeating the photic stress experiment with isolated mouse retina lacking pigmented epithelium, this kind of recovery of the photosensitivity was not observed (FIG. 12). This finding highlights the importance of complexity and interactions between different cell types present in optic vesicles that are functionally integrated within an organoid.

Optic Vesicle Brain Organoids can be Used to Model ZIKV Exposure During Eye Development

Zika virus (ZIKV) has a tropism for neural progenitor cells (NPCs) in developing the brain. Brain organoids can be used to model microcephaly caused by recent ZIKV isolates (Gabriel, E. et al. Recent Zika Virus Isolates Induce Premature Differentiation of Neural Progenitors in Human Brain Organoids. Cell stem cell, (2017); Qian, X. et al. Brain-Region-Specific Organoids Using Mini-bioreactors for Modeling ZIKV Exposure. Cell 165, 1238-1254, (2016)). To date, at least two cases have been reported that Uveitis is associated with ZIKV infection, and a ZIKV protein NS4B interacts with CLN6, a protein that is associated with retinal defects (Furtado, J. M., Esposito, D. L., Klein, T. M., Teixeira-Pinto, T. & da Fonseca, B. A. Uveitis Associated with Zika Virus Infection. The New England journal of medicine 375, 394-396, (2016); Kodati, S. et al. Bilateral posterior uveitis associated with Zika virus infection. Lancet 389, 125-126, (2017); Scaturro, P. et al. An orthogonal proteomic survey uncovers novel Zika virus host factors. Nature 561, 253-257,(2018)). These indications raise the question of whether ZIKV may also have a tropism for retinal cells, an aspect that has not yet tested. As an application of the optic vesicle brain organoids for disease modeling, the inventors tested whether ZIKV has a tropism for retinal progenitors. After a transient two-day ZIKV-AM exposure, the virus could readily infect the optic vesicle region at the vicinity of RPE where RAX-positive progenitor cells are enriched. This suggests a tropism of ZIKVAM for RAX-positive progenitor cells that specify visual fields during eye development (FIG. 13).

Discussion

The examples showed that iPSC-derived brain organoids could assemble optic vesicles and generate diverse retinal cell types organized as a developing stratified neural retina. Due to the assumption that brain organoids are not able to strictly follow self-patterning rules like embryos, brain organoids are instead thought to remain as chaotic 3D tissues lacking anterior-posterior and dorsal-ventral axes. Organoids according to the present invention develop bilaterally symmetric optic vesicles from the forebrain-like region without an artificial induction of signaling centers. While the mechanisms that govern spatial topography to establish bilaterally symmetric optic vesicles require future investigations, the presented work already highlights the intrinsic self-patterning ability of iPSCs in a highly complex biological process.

Earlier works by the Sasai laboratory have pioneered the generation of optic vesicles from human embryonic stem cells, which displayed stratified neural retina after >260 days of culturing (Nakano, T. et al. Self-formation of optic vesicles and storable stratified neural retina from human ESCs. Cell stem cell 10, 771-785, (2012)). A later work by Zhong and colleagues has generated similar retinal organoids and demonstrated that they are light sensitive (Zhong, X. et al. Generation of three-dimensional retinal tissue with functional photoreceptors from human iPSCs. Nature communications 5, 4047, (2014)). Further works have reported that the generation of similar optic vesicles is versatile across several independent pluripotent cell lines (Capowski, E. E. et al. Reproducibility and staging of 3D human retinal organoids across multiple pluripotent stem cell lines. Development 146, (2019)). These works have focused on generating pure retina. Thus, the functional integration of these 3D retinal structures into 3D brain organoid tissues has remained untested. A recent effort in culturing 3D brain organoids for nine months has resulted in the generation of organoids with undefined photosensitive cell types. While these long-term cultured organoids contained diverse cell types and networks, they did not display polarization, stratification, or visibly apparent RPE rich distinct optic vesicles (Risso, D., Ngai, J., Speed, T. P. & Dudoit, S. Normalization of RNA-seq data using factor analysis of control genes or samples. Nature biotechnology 32, 896-902 (2014)).

The brain organoids described here contain bilaterally symmetric optic vesicles constituting cornea, lens, photoreceptors, and retinal pigment epithelia. When optic vesicles were observed in an organoid, their cytoarchitectures and functionality were similar regardless of which cell line was used to generate the organoid. Importantly, the optic vesicles of these organoids are functionally integrated as demonstrated by axon-like projection tracts and electrically active neuronal networks. The time frame to generate these complex structures (from 50 to 60 days) is a crucial step forward in vitro developmental neurobiology as it fulfills the practical feasibility of using this system in multiple experimental setups in a reasonable time window. Importantly, the generation of various cell types organized as an immature stratified neural retina within 50 days parallels with that of human embryo (O'Rahilly, R. The prenatal development of the human eye. Experimental eye research 21, 93-112 (1975)). Further refinement of the described method will help to reconstruct the complexities of early brain development in parallel with optic vesicle morphogenesis and its circuitry.

Optic vesicle brain organoids described here display both neuronal and surface ectodermal sublineages within a single organoid. These aspects are uniquely enabled by the method according to the present invention, allowing comprehensive functional studies in vitro (FIG. 4, panel E). As an example, the inventors successfully applied ERGs, a diagnostic test method, to show strong hyperpolarizing a-wave, which is strictly connected with the early photoreceptor response, after eliminating the b-wave (FIG. 5, panel B). Furthermore, the photic stress experiments indicating that photoreceptors can be regenerated (FIG. 5, panel C). The optic vesicle brain organoids of the present invention are suitable for potential future functional studies related to the development of the visual system. Another important application is to serve the enormous demand for stratified neural epithelia for replacement therapy against retinal degenerative disorders. Optic vesicle brain organoids displaying highly specialized neuronal cell types open up new avenues to further scale up for generating personalized organoids and RPE sheets for transplantation therapies. Indeed, the inventors showed that RPE cells could further be cultured from optic vesicle organoids (FIG. 6).

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element or step not specified in the claim element. As used herein, “consisting essentially of” does not exclude materials or steps that do not materially affect the basic and novel characteristics of the claim. Any recitation of the term “comprising”, particularly in a description of components of a composition or in a description of device elements, is understood to encompass compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

1. A three-dimensional (3D) brain organoid comprising both first cells that are neuronal and second cells that are non-neuronal.

2. The 3D brain organoid according to claim 1, wherein the first cells comprise an optic vesicle structure.

3. The 3D brain organoid according to claim 1, wherein the first cells comprise retinal pigment epithelium (RPE) cells, photoreceptor precursor cells, amacrine cells, bipolar cells, horizontal cells, ganglion cells, Muller cells or combinations thereof.

4. The 3D brain organoid according to claim 1, wherein the first cells express:
(a) a retinal cell marker selected from the group consisting of, OTX, VSX, FOXG1, SOX2, PAX6, APOE, ITM2B, COL9A1, NEFL, POUF1, TFAP2A, ONECUTI, ONECUT2, SIX3, Cone-rod homeobox protein (CRX), Recoverin (RCVRN), Neural retina leucine zipper (NRL), Basic Helix-Loop-Helix Family Member E22 (BHLHE22), PR/SET domain 8 (PRDM8), Protein kinase C alpha (PKCa), Atonal BHLH Transcription Factor 7 (ATOH7), Distal-Less Homeobox 2 (DLX2), Syntaxin 1A (STXIA); and/or
(b) Retinoid Isomerohydrolase (RPE65), cellular retinaldehyde binding protein (CRALBP), ADP Ribosylation Factor Like GTPase 13B (Arl13B), Actin, Bestrophin 1 (BEST1),
(c) Synapsin 1 (SYN1), Myelin binding protein (MBP), class III beta tubulin (TUJ1), Purkinje Cell Protein 4 (PCP4), or
(d) Laminin.

5. The 3D brain organoid according to claim 1, wherein the second cells form at least one lens, at least one cornea, or combinations thereof.

6. The 3D brain organoid according to claim 1, wherein the second cells form eye development cell types.

7. The 3D brain organoid according to claim 1, wherein the second cells form a lateral geneicuoate nucleus.

8. The 3D brain organoid according to claim 1, wherein the second cells form an optic chiasm.

9. The 3D brain organoid according to claim 5, wherein
the at least one lens expresses genes selected from the group of lens markers consisting of CRYAB, CRYBB3, and/or the cornea expresses genes selected from the group of cornea markers consisting of OPTC and KRT3.

10. A method for producing a 3D brain organoid comprising both first cells that are neuronal cells and second cells that are non-neuronal cells, wherein the method comprises the steps of
i) culturing at least one stem cell,
ii) subjecting said at least one stem cell to neural induction leading to neurospheres,
iii) collecting and culturing the neurospheres,
iv) exposing said neurospheres to a composition comprising at least one retinoic acid receptor (RAR) activator, at least one bone morphogenetic protein (BMP) pathway inhibitor and at least one transforming growth factor (TGF) Wactivin/nodal pathway inhibitor,
wherein the at least one stem cell comprises at most 100,000 cells, preferably between 10 and 90,000 cells, more preferably between 100 and 70,000 cells, even more preferably between 1,000 and 50,000 cells, more preferably between 5,000 and 25,000 cells, and wherein said exposure is made within two weeks, preferably within 2 days to 15 days, more preferably within 3 days to 14 days, even more preferred within 5 to 12 days after neural induction.

11. The method according to claim 10, wherein said at least one retinoic acid receptor (RAR) activator is retinol acetate or a derivative thereof, or a semi-synthetic or a synthetic analogue of retinol acetate, or a derivative of such an analogue.

12. The method according to claim 10, wherein said at least one BMP pathway inhibitor is dorsomorphin or a derivative thereof, or a semi-synthetic or a synthetic analogue of dorsomorphin, or a derivative of such an analogue.

13. The method according to claim 10, wherein said at least one TGFβ/activin/nodal pathway inhibitor is SB431542 or a derivative thereof, or a semi-synthetic or a synthetic analogue of SB431542, or a derivative of such an analogue.

14. A 3D brain organoid obtained by the method of claim 10 or tissue, preferably an artificial retinal pigment epithelium (RPE), isolated therefrom.

15. A patch comprising a tissue, preferably an artificial RPE, according to claim 14 and a basement membrane.

16. A 3D brain organoid according to claim 1 for use in the treatment of diseases associated with visual impairment including retinal disorders, the 3D brain organoid or the tissue or the cells preferably being comprised in a pharmaceutical composition.

17. Use of the 3D brain organoid according to claim 1 in toxicity in vitro assays comprising exposing the 3D brain organoid to a bacteria and/or virus.

18. A composition comprising at least one retinoic acid receptor (RAR) activator, at least one bone morphogenetic protein (BMP) pathway inhibitor and at least one transforming growth factor (TGF) Wactivin/nodal pathway inhibitor.
